(19) United States
(12) Patent Application Publication
(10) Pub. No.: US 2015/0265710 A1
(43) Pub. Date: Sep. 24, 2015

(54) Fatty Acid Acylated D-Amino Acids for Oral Peptide Delivery

(71) Applicant: Novo Nordisk A/S, Bagsværd (DK)

(72) Inventors: Frantisek Hubalek, Herlev (DK); Florian Anders Foeger, Malmo (SE); Abdallah Makhlof, Bagsvaerd (DK)

(21) Appl. No.: 14/433,203

(22) PCT Filed: Oct. 16, 2013
(86) PCT No.: PCT/EP2013/071575

§ 371 Date: Apr. 2, 2015

Related U.S. Application Data
(60) Provisional application No. 61/715,412, filed on Oct. 18, 2012.

(30) Foreign Application Priority Data
Oct. 17, 2012 (EP) 12188807.7

(51) Int. Cl. A61K 47/18; A61K 9/107; A61K 47/10; A61K 9/00; A61K 38/28; A61K 47/22

(52) U.S. Cl. CPC A61K 47/183; A61K 38/28; A61K 47/22; A61K 47/10; A61K 9/0053; A61K 9/107; A61K 9/1075

(57) ABSTRACT

The present invention relates to fatty acid acylated amino acids (FA-Daa's) acting as permeation enhancers for oral delivery of therapeutic macromolecules such as peptides and pharmaceutical compositions comprising such FA-Daa's.

### TECHNICAL FIELD

The technical field of this invention relates to fatty acid acylated D-amino acids (FA-Daa's) for oral delivery of therapeutic hydropilic peptides and proteins and pharmaceutical compositions comprising such FA-Daa's.

### BACKGROUND

Many pathological states due to deficiencies in or complete failure of the production of a certain macromolecules (e.g. proteins and peptides) are treated with an invasive and inconvenient parenteral administration of therapeutic macromolecules, such as hydrophilic peptides or proteins. One example hereof is the administration of insulin in the treatment of insulin dependent patients, who are in need of one or more daily doses of insulin. The oral route is desirable for administration due to its non-invasive nature and has a great potential to decrease the patient's discomfort related to drug administration and to increase drug compliance. However several barriers exist; such as the enzymatic degradation in the gastrointestinal (GI) tract, drug efflux pumps, insufficient and variable absorption from the intestinal mucosa, as well as first pass metabolism in the liver and until now no products for oral delivery of therapeutic hydrophilic proteins are found to be marketed.

The research into new surfactants with low irritant action has lead to the development of different surfactants derived from amino acids (Mitjans et al., 2003; Benavides et al., 2004; Sanchez et al., 2006) FA-Daa's are amino acid based surfactants and thus mild biodegradable surfactants with a low toxicity.

Foger et al. described the impact of the molecular weight on oral absorption of hydrophilic peptide drugs and showed that the permeability decreased with increasing molecular weight of such hydrophilic peptide drugs (Amino Acids (2008) 25: 233-241, DOI 10.1007/s00726-007-0581-5).

A non-limiting example of a hydrophilic proteins and polypeptides is human insulin which ais degraded by various digestive enzymes found in the stomach (pepsin), in the intestinal lumen (chymotrypsin, trypsin, elastase, carboxypeptidases, etc.) and in the mucosal surfaces of the GI tract (aminopeptidases, carboxypeptidases, enteropeptidases, dipeptidyl peptidases, endopeptidases, etc.).

WO2004147578 relates to fatty acid acylated amino acids used as permeation enhancers for uncharged molecules including uncharged macromolecules such as cyclosporine.

WO2001035998 relates to acylated amino acids used as transdermal and transmucosal absorption promoters for macromolecules, such as hydrophilic peptides or proteins.

WO2004064758 relates to an oral composition for delivering pharmaceutical peptides, such as insulin, growth hormone and GLP-1, comprising absorption enhancers, including acyl amino acids.

US2005282756 is related to a dry powder composition comprising insulin and an absorption enhancer.

WO2003030865 is related to insulin compositions comprising surfactants such as ionic surfactants and does also contain oil or lipid compounds such as triglycerides and does further comprise long chain esterified fatty acids (C12 to C18).

WO2004064758 is related to an oral pharmaceutical composition for delivering pharmaceutical peptides, comprising absorption enhancers.

The oral route of administration is rather complex and a need for establishment of an acceptable composition suitable for the treatment of patients, with an effective bioavailability of the macromolecule, such as hydrophilic peptides or proteins, is existent.

### SUMMARY

This invention regards a pharmaceutical composition comprising at least one fatty acid acylated D-amino acid (FA-Daa) or salt thereof and a hydrophilic peptide or protein, wherein the amino acid moiety of said FA-Daa is selected from non-polar uncharged amino acids or acidic amino acids, wherein the stereo configuration of the chiral carbon atom in said amino acid moiety is D and the fatty acid moiety of said FA-Daa is attached by acylation to the alpha amino group of said amino acid moiety and comprises 12, 14, 16 or 18 carbon atoms, when said amino acid moiety is from a non-polar uncharged amino acid and 16 or 18, when said amino acid moiety is from an acidic amino acid.

Methods for manufacture of FA-Daa according to the present invention and pharmaceutical compositions comprising such FA-Daa are also subject to the present invention.

Further the invention regards a method for increasing the bioavailability of insulin, insulin peptides or proteins, insulin analogues or insulin derivatives comprising the steps of including a FA-Daa in a pharmaceutical composition insulin, insulin peptides or proteins, insulin analogues or insulin derivatives administered to an individual.

### DESCRIPTION

The present invention is related to oral permeation enhancers based on D-isoforms of amino acids. The present invention is related to oral permeation enhancers based on D-isoforms of charged, uncharged or acidic amino acids. The present invention is related to oral permeation enhancers based on D-isoforms of non-polar uncharged or acidic amino acids and are in this application refered to as Fatty acid N-acylated D-amino acid (FA-Daa) or Fatty acid acylated D-amino acid (FA-Daa).

The present invention also relates to oral permeation enhancers based in D-isomers of amino acids, used for enhancing the permeation of orally administered hydrophilic peptides. The present invention also relates to oral permeation enhancers based in D-isomers of amino acids, used for enhancing the permeation of orally administered insulin peptides. The present invention also relates to oral permeation enhancers based in D-isomers of amino acids, used for enhancing the permeation of orally administered insulin peptides or proteins, such as insulin analogues or insulin derivatives.

The present invention also relates to oral permeation enhancers based in D-isomers of charged amino acids, used for enhancing the permeation of orally administered hydrophilic peptides. The present invention also relates to oral permeation enhancers based in D-isomers of charged amino acids, used for enhancing the permeation of orally administered insulin peptides or proteins. The present invention also relates to oral permeation enhancers based in D-isomers of charged amino acids, used for enhancing the permeation of orally administered insulin peptides or proteins, such as insulin analogues or insulin derivatives.

The present invention also relates to oral permeation enhancers based in D-isomers of acidic amino acids, used for enhancing the permeation of orally administered hydrophilic peptides. The present invention also relates to oral permeation enhancers based in D-isomers of acidic amino acids, used for enhancing the permeation of orally administered insulin peptides or proteins. The present invention also relates to oral permeation enhancers based in D-isomers of acidic amino acids, used for enhancing the permeation of orally administered insulin peptides or proteins, such as insulin analogues or insulin derivatives.

The present invention relates to oral permeation enhancers based on D-isomers of charged amino acids in a pharmaceutical composition. The present invention relates to oral permeation enhancers based on D-isomers of charged amino acids in a pharmaceutical composition further comprising hydrophilic peptides or proteins. The present invention relates to oral permeation enhancers based on D-isomers of charged amino acids in a pharmaceutical composition further comprising hydrophilic peptides or proteins, such as insulin analogues or insulin peptides. The present invention is related to pharmaceutical compositions, comprising FA-Daa's acting as permeation enhancers suitable for oral administration of therapeutic macromolecules (e.i. therapeutic active peptides and proteins). More specifically therapeutic macromolecules, such as hydrophilic peptides or proteins according to the present invention are hydrophilic peptides and proteins which have a therapeutical activity and include but are not limited to insulin. It has surprisingly been found that at least one FA-Daa or a salt thereof represented by the general formula A-Xy, wherein A is a non-polar uncharged or acidic amino acid and Xy is a fatty acid moiety attached by acylation to A's alpha amino group and y represents the number of carbon atoms in said fatty acid moiety, wherein y is 12, 14, 16 or 18 when said amino acid is a non-polar uncharged amino acid and y is 16 or 18 when said amino acid is an acidic, wherein the stereo configuration of the chiral carbon atom in the amino acid moiety is D is better absorption enhancer of hydrophillic peptides, such as insulin peptides and proteins when compared to absorption enhancement of their L-isomer counterparts.

Due to their low toxicity and increasing effect on oral bioavailability of the therapeutic macromolecule, such as a hydrophilic peptide or protein, FA-Daa's according to the present invention are valuable ingredients in oral pharmaceutical compositions. Especially valuable are FA-Daa's according to this invention in oral pharmaceutical compositions comprising hydrophilic peptides or proteins as active ingredient. This is of interest for diseases that demand chronic administration of therapeutic macromolecules (e.g. peptides or proteins), but is not limited hereto, since the most non-invasive, non-toxic administration of drugs is generally favoured in any treatment, also for sporadic or bulk administration of therapeutics. So far, there are no commercial hydrophilic proteins available as oral formulation mainly due to the great challenges of enzymatic degradation and very low intestinal permeability of such hydrophilic proteins and peptides.

The invention may also solve further problems that will be apparent from the disclosure of the exemplary aspects. The present invention is related to oral pharmaceutical compositions comprising FA-Daa's suitable for increasing the bioavailability of therapeutic macromolecules (e.g. peptides and proteins) and their absorption.

One aspect of the invention is a pharmaceutical composition comprising at least one therapeutic macromolecule, such as hydrophilic peptides or proteins and at least one FA-Daa. One aspect of the invention is a pharmaceutical composition comprising at least one therapeutic peptide or protein and at least one FA-Daa, wherein said therapeutic peptide or protein is a hydrophilic peptide or protein.

One aspect of the invention is a pharmaceutical composition comprising at least one therapeutic peptide and at least one FA-Daa and a hydrophilic peptide or protein.

One aspect of the invention is a pharmaceutical composition comprising at least one therapeutic peptide and at least one FA-Daa and a hydrophilic peptide or protein, wherein said hydrophilic peptide or protein is insulin.

One aspect of the invention is a pharmaceutical composition comprising at least one therapeutic peptide and at least one FA-Daa and at least one hydrophilic peptide or protein.

One aspect of the invention is a pharmaceutical composition comprising at least one therapeutic peptide and at least one FA-Daa and at least one hydrophilic peptide or protein, wherein said hydrophilic peptide or protein is insulin.

One aspect of the invention is a pharmaceutical composition comprising at least one therapeutic peptide and at least one FA-Daa and at least one hydrophilic peptide or protein, wherein said hydrophilic peptide or protein is insulin, an insulin analogue or a derivatised insulin peptide or protein.

One aspect of the invention is a pharmaceutical composition comprising at least one therapeutic peptide and at least one FA-Daa and at least one hydrophilic peptide or protein, wherein said hydrophilic peptide or protein is insulin, an insulin analogue.

One aspect of the invention is a pharmaceutical composition comprising at least one therapeutic peptide and at least one FA-Daa and at least one hydrophilic peptide or protein, wherein said hydrophilic peptide or protein is insulin, a derivatised insulin peptide or protein.

In one aspect of the invention, the pharmaceutical composition comprises at least one therapeutic macromolecule and one or more FA-Daa, based on a non-polar uncharged D-amino acid. In one aspect of the invention, the pharmaceutical composition comprises at least one therapeutic macromolecule and one or more FA-Daa, based on an acidic D-amino acid.

In one aspect of the invention, the pharmaceutical composition comprises at least one therapeutic peptide or protein and one or more FA-Daa, based on a non-polar uncharged D-amino acid, said one or more non-polar uncharged D-amino acid may be selected from the group consisting of Alanine (Ala, A), Isoleucine (Ile, I), Leucine (Leu, L), Proline (Pro, P) and Valine (Val, V).

In one aspect of the invention, the pharmaceutical composition comprises at least one therapeutic peptide or protein and one or more FA-Daa, based on an acidic D-amino acid, said one or more acidic D-amino acid may be selected from the group consisting of Aspartic acid (Asp) and Glutamic acid (Glu).

In one aspect of the invention, the pharmaceutical composition comprises at least one therapeutic peptide or protein and one or more FA-Daa, based on a non-polar uncharged D-amino acid and a fatty acid moiety consisting of 12 to 18 carbon atoms.

In one aspect of the invention, the pharmaceutical composition comprises at least one therapeutic peptide or protein and one or more FA-Daa, based on a non-polar uncharged D-amino acid and a fatty acid moiety consisting of 14 to 18 carbon atoms.

In one aspect of the invention, the pharmaceutical composition comprises at least one therapeutic peptide or protein and one or more FA-Daa, based on a non-polar uncharged D-amino acid and a fatty acid moiety consisting of 16 to 18 carbon atoms.

In one aspect of the invention, the pharmaceutical composition comprises at least one therapeutic peptide or protein and one or more FA-Daa, based on an acidic D-amino acid and a fatty acid moiety consisting of 12 to 18 carbon atoms.

In one aspect of the invention, the pharmaceutical composition comprises at least one therapeutic peptide or protein and one or more FA-Daa, based on an acidic D-amino acid and a fatty acid moiety consisting of 16 to 18 carbon atoms.

In one aspect of the invention, the pharmaceutical composition comprises at least one therapeutic peptide or protein and one or more FA-Daa, based on an acidic D-amino acid and a fatty acid moiety consisting of 16 atoms.

In one aspect of the invention, the pharmaceutical composition comprises at least one therapeutic peptide or protein and one or more FA-Daa, based on an acidic D-amino acid and a fatty acid moiety consisting of 18 carbon atoms.

In one aspect of the invention, the pharmaceutical composition comprises at least one therapeutic peptide or protein and one or more FA-Daa, wherein at least one FA-Daa is based on D-Alanine and a fatty acid moiety consisting of 12 to 18 carbon atoms. In one aspect of the invention, the pharmaceutical composition comprises at least one therapeutic peptide or protein and one or more FA-Daa, wherein at least one FA-Daa is based on D-Alanine and a fatty acid moiety consisting of 14 to 18 carbon atoms. In one aspect of the invention, the pharmaceutical composition comprises at least one therapeutic peptide or protein and one or more FA-Daa, wherein at least one FA-Daa is based on D-Alanine and a fatty acid moiety consisting of 16 to 18 carbon atoms. In one aspect of the invention, the pharmaceutical composition comprises at least one therapeutic peptide or protein and one or more FA-Daa, wherein at least one FA-Daa is based on D-Alanine and a fatty acid moiety consisting of 12, 14, 16 or 18 carbon atoms. In one aspect of the invention, the pharmaceutical composition comprises at least one therapeutic peptide or protein and one or more FA-Daa, wherein at least one FA-Daa is based on D-Alanine and a fatty acid moiety consisting of 12 or 14 carbon atoms.

In one aspect of the invention, the pharmaceutical composition comprises at least one therapeutic peptide or protein and one or more FA-Daa, based on a non-polar uncharged D-amino acid and a fatty acid moiety consisting of 12 or 14 carbon atoms. In one aspect of the invention, the pharmaceutical composition comprises at least one therapeutic peptide or protein and one or more FA-Daa, based on a non-polar uncharged D-amino acid and a fatty acid moiety consisting of 16 or 18 carbon atoms.

In one aspect of the invention, the pharmaceutical composition comprises at least one therapeutic peptide or protein and one or more FA-Daa, wherein at least one FA-Daa is based on D-Isoleucine and a fatty acid moiety consisting of 12 to 18 carbon atoms. In one aspect of the invention, the pharmaceutical composition comprises at least one therapeutic peptide or protein and one or more FA-Daa, wherein at least one FA-Daa is based on D-Isoleucine and a fatty acid moiety consisting of 14 to 18 carbon atoms. In one aspect of the invention, the pharmaceutical composition comprises at least one therapeutic peptide or protein and one or more FA-Daa, wherein at least one FA-Daa is based on D-Isoleucine and a fatty acid moiety consisting of 16 to 18 carbon atoms. In one aspect of the invention, the pharmaceutical composition comprises at least one therapeutic peptide or protein and one or more FA-Daa, wherein at least one FA-Daa is based on D-Isoleucine and a fatty acid moiety consisting of 12, 14, 16 or 18 carbon atoms. In one aspect of the invention, the pharmaceutical composition comprises at least one therapeutic peptide or protein and one or more FA-Daa, wherein at least one FA-Daa is based on D-Isoleucine and a fatty acid moiety consisting of 12 or 14 carbon atoms.

In one aspect of the invention, the pharmaceutical composition comprises at least one therapeutic peptide or protein and one or more FA-Daa, wherein at least one FA-Daa is based on D-Leucine and a fatty acid moiety consisting of 12 to 18 carbon atoms. In one aspect of the invention, the pharmaceutical composition comprises at least one therapeutic peptide or protein and one or more FA-Daa, wherein at least one FA-Daa is based on D-Leucine and a fatty acid moiety consisting of 14 to 18 carbon atoms. In one aspect of the invention, the pharmaceutical composition comprises at least one therapeutic peptide or protein and one or more FA-Daa, wherein at least one FA-Daa is based on D-Leucine and a fatty acid moiety consisting of 16 to 18 carbon atoms. In one aspect of the invention, the pharmaceutical composition comprises at least one therapeutic peptide or protein and one or more FA-Daa, wherein at least one FA-Daa is based on D-Leucine and a fatty acid moiety consisting of 12, 14, 16 or 18 carbon atoms. In one aspect of the invention, the pharmaceutical composition comprises at least one therapeutic peptide or protein and one or more FA-Daa, wherein at least one FA-Daa is based on D-Leucine and a fatty acid moiety consisting of 12 or 14 carbon atoms. In one aspect of the invention, the pharmaceutical composition comprises at least one therapeutic peptide or protein and one or more FA-Daa, wherein at least one FA-Daa is based on D-Leucine and a fatty acid moiety consisting of 12 carbon atoms.

In one aspect of the invention, the pharmaceutical composition comprises at least one therapeutic peptide or protein and one or more FA-Daa, wherein at least one FA-Daa is based on D-Valine and a fatty acid moiety consisting of 12 to 18 carbon atoms. In one aspect of the invention, the pharmaceutical composition comprises at least one therapeutic peptide or protein and one or more FA-Daa, wherein at least one FA-Daa is based on D-Valine and a fatty acid moiety consisting of 14 to 18 carbon atoms. In one aspect of the invention, the pharmaceutical composition comprises at least one therapeutic peptide or protein and one or more FA-Daa, wherein at least one FA-Daa is based on D-Valine and a fatty acid moiety consisting of 16 to 18 carbon atoms. In one aspect of the invention, the pharmaceutical composition comprises at least one therapeutic peptide or protein and one or more FA-Daa, wherein at least one FA-Daa is based on D-Valine and a fatty acid moiety consisting of 12, 14, 16 or 18 carbon atoms. In one aspect of the invention, the pharmaceutical composition comprises at least one therapeutic peptide or protein and one or more FA-Daa, wherein at least one FA-Daa is based on D-Valine and a fatty acid moiety consisting of 12 or 14 carbon atoms.

In one aspect of the invention, the pharmaceutical composition comprises at least one therapeutic peptide or protein and one or more FA-Daa, wherein at least one FA-Daa is based on D-Proline and a fatty acid moiety consisting of 12 to 18 carbon atoms. In one aspect of the invention, the pharmaceutical composition comprises at least one therapeutic peptide or protein and one or more FA-Daa, wherein at least one FA-Daa is based on D-Proline and a fatty acid moiety consisting of 14 to 18 carbon atoms. In one aspect of the invention, the pharmaceutical composition comprises at least one therapeutic peptide or protein and one or more FA-Daa, wherein at least one FA-Daa is based on D-Proline and a fatty acid moiety consisting of 16 to 18 carbon atoms. In one aspect of the invention, the pharmaceutical composition comprises at least one therapeutic peptide or protein and one or more FA-Daa, wherein at least one FA-Daa is based on D-Proline and a fatty acid moiety consisting of 12, 14, 16 or 18 carbon atoms. In one aspect of the invention, the pharmaceutical composition comprises at least one therapeutic peptide or protein and one or more FA-Daa, wherein at least one FA-Daa is based on D-Proline and a fatty acid moiety consisting of 12 or 14 carbon atoms.

In one aspect of the invention, the pharmaceutical composition comprises at least one therapeutic peptide or protein and one or more FA-Daa, wherein at least one FA-Daa is based on D-Aspartic acid and a fatty acid moiety consisting of 16 to 18 carbon atoms. In one aspect of the invention, the pharmaceutical composition comprises at least one therapeutic peptide or protein and one or more FA-Daa, wherein at least one FA-Daa is based on D-Aspartic acid and a fatty acid moiety consisting of 16 or 18 carbon atoms. In one aspect of the invention, the pharmaceutical composition comprises at least one therapeutic peptide or protein and one or more FA-Daa, wherein at least one FA-Daa is based on D-Aspartic acid and a fatty acid moiety consisting of 16 carbon atoms. In one aspect of the invention, the pharmaceutical composition comprises at least one therapeutic peptide or protein and one or more FA-Daa, wherein at least one FA-Daa is based on D-Aspartic acid and a fatty acid moiety consisting of 18 carbon atoms.

In one aspect of the invention, the pharmaceutical composition comprises at least one therapeutic peptide or protein and one or more FA-Daa, wherein at least one FA-Daa is based on D-Glutamic acid and a fatty acid moiety consisting of 16 to 18 carbon atoms. In one aspect of the invention, the pharmaceutical composition comprises at least one therapeutic peptide or protein and one or more FA-Daa, wherein at least one FA-Daa is based on D-Glutamic acid and a fatty acid moiety consisting of 16 or 18 carbon atoms. In one aspect of the invention, the pharmaceutical composition comprises at least one therapeutic peptide or protein and one or more FA-Daa, wherein at least one FA-Daa is based on D-Glutamic acid and a fatty acid moiety consisting of 16 carbon atoms. In one aspect of the invention, the pharmaceutical composition comprises at least one therapeutic peptide or protein and one or more FA-Daa, wherein at least one FA-Daa is based on D-Glutamic acid and a fatty acid moiety consisting of 18 carbon atoms.

In one aspect of the invention, the pharmaceutical composition comprises at least one therapeutic peptide or protein and one or more FA-Daa, based on a non-polar uncharged D-amino acid and a fatty acid moiety consisting of 8 carbon atoms. In one aspect of the invention, the pharmaceutical composition comprises at least one therapeutic peptide or protein and one or more FA-Daa, based on a non-polar uncharged D-amino acid and a fatty acid moiety consisting of 10 carbon atoms. In one aspect of the invention, the pharmaceutical composition comprises at least one therapeutic peptide or protein and one or more FA-Daa, based on a non-polar uncharged D-amino acid and a fatty acid moiety consisting of 12 carbon atoms. In one aspect of the invention, the pharmaceutical composition comprises at least one therapeutic peptide or protein and one or more FA-Daa, based on a non-polar uncharged D-amino acid and a fatty acid moiety consisting of 14 carbon atoms. In one aspect of the invention, the pharmaceutical composition comprises at least one therapeutic peptide or protein and one or more FA-Daa, based on a non-polar uncharged D-amino acid and a fatty acid moiety consisting of 16 carbon atoms. In one aspect of the invention, the pharmaceutical composition comprises at least one therapeutic peptide or protein and one or more FA-Daa, based on a non-polar uncharged D-amino acid and a fatty acid moiety consisting of 18 carbon atoms.

In one aspect of the invention, the pharmaceutical composition comprises at least one therapeutic peptide or protein and one or more FA-Daa, based on a polar uncharged D-amino acid and a fatty acid moiety consisting of 8 carbon atoms. In one aspect of the invention, the pharmaceutical composition comprises at least one therapeutic peptide or protein and one or more FA-Daa, based on a polar uncharged D-amino acid and a fatty acid moiety consisting of 10 carbon atoms. In one aspect of the invention, the pharmaceutical composition comprises at least one therapeutic peptide or protein and one or more FA-Daa, based on a polar uncharged D-amino acid and a fatty acid moiety consisting of 12 carbon atoms. In one aspect of the invention, the pharmaceutical composition comprises at least one therapeutic peptide or protein and one or more FA-Daa, based on a polar uncharged D-amino acid and a fatty acid moiety consisting of 14 carbon atoms. In one aspect of the invention, the pharmaceutical composition comprises at least one therapeutic peptide or protein and one or more FA-Daa, based on a polar uncharged D-amino acid and a fatty acid moiety consisting of 16 carbon atoms. In one aspect of the invention, the pharmaceutical composition comprises at least one therapeutic peptide or protein and one or more FA-Daa, based on a polar uncharged D-amino acid and a fatty acid moiety consisting of 18 carbon atoms.

In one aspect of the invention, the pharmaceutical composition comprises at least one therapeutic peptide or protein and one or more FA-Daa, based on an acidic D-amino acid and a fatty acid moiety consisting of 8 carbon atoms. In one aspect of the invention, the pharmaceutical composition comprises at least one therapeutic peptide or protein and one or more FA-Daa, based on an acidic D-amino acid and a fatty acid moiety consisting of 10 carbon atoms. In one aspect of the invention, the pharmaceutical composition comprises at least one therapeutic peptide or protein and one or more FA-Daa, based on an acidic D-amino acid and a fatty acid moiety consisting of 12 carbon atoms. In one aspect of the invention, the pharmaceutical composition comprises at least one therapeutic peptide or protein and one or more FA-Daa, based on an acidic D-amino acid and a fatty acid moiety consisting of 14 carbon atoms. In one aspect of the invention, the pharmaceutical composition comprises at least one therapeutic peptide or protein and one or more FA-Daa, based on an acidic D-amino acid and a fatty acid moiety consisting of 16 carbon atoms. In one aspect of the invention, the pharmaceutical composition comprises at least one therapeutic peptide or protein and one or more FA-Daa, based on an acidic D-amino acid and a fatty acid moiety consisting of 18 carbon atoms.

In one aspect of the invention, the pharmaceutical composition comprises at least one therapeutic peptide or protein and one or more FA-Daa, based on a basic D-amino acid and a fatty acid moiety consisting of 8 carbon atoms. In one aspect of the invention, the pharmaceutical composition comprises at least one therapeutic peptide or protein and one or more FA-Daa, based on a basic D-amino acid and a fatty acid moiety consisting of 10 carbon atoms. In one aspect of the invention, the pharmaceutical composition comprises at least one therapeutic peptide or protein and one or more FA-Daa, based on a basic D-amino acid and a fatty acid moiety consisting of 12 carbon atoms. In one aspect of the invention, the pharmaceutical composition comprises at least one therapeutic peptide or protein and one or more FA-Daa, based on a basic D-amino acid and a fatty acid moiety consisting of 14 carbon atoms. In one aspect of the invention, the pharmaceutical composition comprises at least one therapeutic peptide or protein and one or more FA-Daa, based on a basic D-amino acid and a fatty acid moiety consisting of 16 carbon atoms. In one aspect of the invention, the pharmaceutical composition comprises at least one therapeutic peptide or protein and one or more FA-Daa, based on a basic D-amino acid and a fatty acid moiety consisting of 18 carbon atoms.

In one aspect of the invention, the pharmaceutical composition comprises at least one therapeutic peptide or protein, such as a hydrophilic peptide or protein and FA-Daa's, based on a mixture of FA-Daa's.

In one aspect a pharmaceutical composition according to the present invention comprises one or more commercially available FA-Daa's.

According to this invention a FA-Daa comprises an amino acid and a fatty acid attached to the amino acid by acylation of said amino acid's alpha-amino group. According to this invention a FA-Daa comprises an amino acid and a fatty acid side chain (i.e. a fatty acid moiety) attached to the amino acid by acylation of said amino acid's alpha-amino group. According to this invention a FA-Daa comprises an amino acid and a fatty acid moiety attached to the amino acid by acylation of said amino acid's alpha-amino group. In one aspect a FA-Daa according to this invention comprises an acylated amino acid, wherein the fatty acid side chain (i.e the fatty acid moiety) of an FA-Daa according to the present invention is located at the alpha amino group of the amino acid

In one aspect the FA-Daa according to the present invention can be prepared by known methods in the art. In one aspect the α-carboxyl group and reactive side chain groups of the amino acid is protected prior to coupling of activated fatty acid to the N-teminal amino group. Non-limiting examples of such methods are given in the Example section.

In one aspect the present invention is a method for the manufacture of compositions comprising FA-Daa, comprising the manufacture of liquid formulations comprising insulin, SEDDS, SMEDDS or SNEDDS formulations were prepared according to the guidance given in WO08145728 example 1 and 2, pages 53-54 wherein the FA-Daa according to this invention are added to the isulin solution.

In one aspect the present invention is a method for the manufacture of compositions comprising FA-Daa insulin, SEDDS, SMEDDS or SNEDDS, comprising the steps: dissolving insulin in a solvent, such as propylene glycol, water and/or glycerol), dissolve a FA-Daa according to the present invention in said insulin solution, whereupon the lipid phase components of SEDDS, SMEDDS or SNEDDS are added to this mixture followed by the surfactants.

In one aspect the present invention is a method for the manufacture of compositions comprising FA-Daa insulin, SEDDS, SMEDDS or SNEDDS, comprising the steps: dissolving insulin in a solvent, such as propylene glycol, water and/or glycerol), dissolve a FA-Daa according to the present invention in said insulin solution, whereupon the components of SEDDS, SMEDDS or SNEDDS are added.

One aspect of the present invention is a method for the manufacture of compositions according to the present invention comprising the step of dissolving insulin in propylene glycol.

One aspect of the present invention is a method for the manufacture of compositions according to the present invention comprising the step of mixing said FA-Daa to a mixture of an insulin peptide or protein and the ingredients for SEDDS, SMEDDS or SNEDDS.

In one aspect the FA-Daa according to the present invention can be prepared by a method comprising at least one of the following steps:
1. Pyridine is added dropwise to a mixture of D-Amino acid and trimethylsilyl chloride in dry dichloromethane. The resulting solution (A) is stirred, optionally overnight.
2. Said solution (A) is cooled to about 0° C., optionally in a cooling bath.
3. A solution (B) of fatty acid chloride in dry dichloromethane is added dropwise to the cooled solution (A).
4. The cooling bath is removed and the mixture of the solutions (A+B) is stirred at room temperature.
5. Hydrochloric acid is added to the solution (A+B) and the mixture is stirred until a pale yellow solid precipitate is formed.
6. The resulting crystals are filtered off and the filtrate is washed with hydrochloric acid and dried, optionally the drying is performed over anhydrous sodium sulfate and evaporation.
7. The residue is combined with previous crystals, dissolved in dichloromethane and crystallized from diethylether and hexanes mixture.
8. The product is filtered off, washed with diethylether and dried, optionally in vacuo result in the desired N-fatty acid D-amino acid as white crystals or oil.

In one aspect the FA-Daa according to the present invention can be prepared by a method comprising at least one of the following steps:
1. Pyridine (7.50 mmol) is added dropwise to a mixture of D-Amino acid (2.28 mmol) and trimethylsilyl chloride in dry dichloromethane (15 mL). The resulting solution (A) is stirred, optionally overnight.
2. Said solution (A) is cooled to 0° C., optionally in a cooling bath.
3. A solution (B) of fatty acid chloride (2.50 mmol) in dry dichloromethane (5 mL) is added dropwise to the cooled solution (A).
4. The cooling bath is removed and the mixture of the solutions (A+B) is stirred for 1.5 hr at room temperature.
5. 1 M Hydrochloric acid (20 mL) is added to the solution (A+B) and the mixture is stirred for 15 min and pale yellow solid precipitate is formed.
6. The resulting crystals are filtered off and washed with 1 M hydrochloric acid (3×20 mL) and dried optionally said drying is performed over anhydrous sodium sulfate and evaporation.
7. The residue is combined with previous crystals, dissolved in dichloromethane and crystallized from diethylether (10 mL) and hexanes (15 mL) mixture.
8. The product is filtered off, washed with diethylether and dried, optionally in vacuo result in the desired N-fatty acid D-amino acid as white crystals or oil.

In one aspect the FA-Daa according to the present invention can be prepared by any method known by the person skilled in the art used in peptide synthesis.

In one aspect the FA-Daa according to the present invention can be prepared by any method known by the person skilled in the art used in peptide synthesis, more specifically known as acylation.

In one aspect the FA-Daa according to the present invention can be prepared by a method comprising at least one of the following steps:
1. Resin (C) preparation.
2. Coupling of Fmoc-protected D-amino acid to said resin resulting in C-Daa-Fmoc, wherein C represents a resin, Daa represents any D-amino acid according to the present invention and Fmoc represents the Fmoc group.
3. Deprotection of Fmoc-Daa on resin resulting in C-Daa, wherein C represents a resin and Daa represents any D-amino acid according to the present invention.
4. Coupling of (C-Daa) and fatty acids according to the present invention, resulting in a C-Daa-FA, wherein C represents a resin, Daa represents any D-amino acid according to the present invention and FA represent any Fatty acid according to the present invention.
5. Decoupling of FA-Daa (which according to the present invention is the same as Daa-FA) from C-Daa-FA.
6. Filtration and washes in between the separate steps and in the end of the procedure by methods well known by the skilled person in the art.
7. Finally a drying of the final FA-Daa product, which will appear as a powder or oil.
   In one aspect the FA-Daa according to the present invention can be prepared by a method comprising at least one of the following steps:
1. A resin (C) mesh is left to swell in dry dichloromethane.
2. A solution of Fmoc-D-amino acid-OH (Daa-Fmoc) and N,N-diisopropylethylamine in dry dichloromethane is added to resin (C) and the mixture is shaken for 4 hrs, resulting in a coupling of said resin and Fatty D-amino acid (C-Daa-Fmoc).
3. Said resin (C-Daa-Fmoc) is filtered and treated with a solution of N,N-diisopropylethylamine in methanol/dichloromethane mixture.
4. Then resin (C-Daa-Fmoc) is washed with N,N-dimethylformamide, and N,N-dimethylformamide.
5. Said Fmoc group is removed from the D-amino acid coupled to the resin (C-Daa-Fmoc) by treatment with 20% piperidine in dimethylformamide.
6. The resulting resin-D-amino acid (C-Daa) is washed with N,N-dimethylformamide, 2-propanol and dichloromethane
7. A solution of fatty acids (FA) according to the present invention (2.22 mmol), ethyl cyano-glyoxylate-2-oxime 2,4,6-collidine and N,N-diisopropylcarbodiimide in dichloromethane/N,N-dimethylformamide mixture is added to resin (C-Daa) and the mixture is shaken for 1.5 hr, resulting in a coupling of said resin coupled to said D amino acid (C-Daa) to said Fatty acid (FA), i.e. (C-FA-Daa).
8. Said Resin product (C-FA-Daa) is filtered and washed with N,N-dimethylformamide, dichloromethane, methanol, dichloromethane and diethylether.
9. The FA-Daa product is cleaved from said Product Resin (C-FA-Daa) by treatment with a mixture of trifluoroacetic acid:triethylsilane:water for 30 minutes.
10. The FA-Daa product is filtered off and washed with trifluoroacetic acid/dichloromethane and dichloromethane.
11. The solvents are removed.
12. The FA-Daa product is dissolved in toluene and the solvent is removed.
13. This procedure of step 12 is repeated ten times to remove the traces of trifluoroacetic acid.
14. A crude product comprising said FA-Daa is dissolved in dichloromethane (5 mL) and diethylether is added to the solution to precipitate the product which is collected by filtration, washed with diethylether and dried in vacuo to yield title compound as brownish powder or an oil.

In one aspect the FA-Daa according to the present invention can be prepared by a method comprising at least one of the following steps:
1. 2-Chlorotrityl resin (C) 100-200 mesh 1.5 mmol/g (1.48 g, 2.22 mmol) is left to swell in dry dichloromethane (10 mL) for 20 min.
2. A solution of Fmoc-D-amino acid-OH (Daa) according to the present invention (1.48 mmol) and N,N-diisopropylethylamine (0.98 mL, 5.62 mmol) in dry dichloromethane (5 mL) is added to resin (C) and the mixture is shaken for 4 hrs, resulting in a coupling of said resin and Fatty D-amino acid (C-Daa)
3. Said resin (C-Daa) is filtered and washed with a solution of N,N-diisopropylethylamine (0.52 mL, 2.96 mmol) in methanol/dichloromethane mixture (4:1, 10 mL, 2×5 min).
4. Then resin (C-Daa) is washed with N,N-dimethylformamide (2×10 mL), dichloromethane (2×10 mL) and N,N-dimethylformamide (3×10 mL).
5. Fmoc group is removed by treatment with 20% piperidine in dimethylformamide (1×5 min, 1×30 min, 2×10 mL).
6. Said resin (C-Daa) is washed with N,N-dimethylformamide (3×10 mL), 2-propanol (2×10 mL) and dichloromethane (20 mL, 2×10 mL).
7. A solution of fatty acids (FA) according to the present invention (2.22 mmol), ethyl cyano-glyoxylate-2-oxime (OXYMA, 0.32 g, 2.22 mmol) 2,4,6-collidine (0.52 mL, 4.00 mmol) and N,N-diisopropylcarbodiimide (0.35 mL, 2.22 mmol) in dichloromethane/N,N-dimethylformamide mixture (4:1, 10 mL) is added to resin (C-Daa) and the mixture is shaken for 1.5 hr, resulting in a coupling of said resin coupled to said D amino acid (C-Daa) to said Fatty acid (FA), i.e. (C-FA-Daa).
8. Said Resin product (C-FA-Daa) is filtered and washed with N,N-dimethylformamide (6×10 mL), dichloromethane (6×10 mL), methanol (6×10 mL), dichloromethane (12×10 mL) and diethylether (3×10 mL).
9. The FA-Daa product is cleaved from said Prosuct Resin (C-FA-Daa) by treatment with a mixture of trifluoroacetic acid:triethylsilane:water (30 mL, 9.25:0.5:0.25) for 30 minutes.
10. The FA-Daa product is filtered off and washed with trifluoroacetic acid/dichloromethane (1:1, 15 mL) and dichloromethane (5×10 mL).
11. The solvents are removed.
12. The FA-Daa product is dissolved in toluene (15 mL) and the solvent is removed.
13. This procedure of step 12 is repeated ten times to remove the traces of trifluoroacetic acid.
14. A crude product comprising said FA-Daa is dissolved in dichloromethane (5 mL) and diethylether (70 mL) is added to the solution to precipitate the product which is collected by filtration, washed with diethylether and dried in vacuo to yield title compound as brownish powder or an oil.

In one aspect, an amino acid according to this invention includes the form of its free acid or a salt. In one aspect an amino acid according to this invention includes the form of its free acid or sodium (Na+) salt. In one aspect an amino acid according to this invention includes the form of its free acid or potassium (K+) salt.

In one aspect a FA-Daa according to this invention comprise amino acid residues in the form of their free acid or a salt. In one aspect a FA-Daa according to this invention comprises amino acid residues in the form of their free acid or sodium (Na+) salt. In one aspect a FA-Daa according to this invention comprises amino acid residues in the form of their free acid or sodium (K+) salt.

In one aspect a FA-Daa according to this invention is soluble at pH values found in GI-tract. In one aspect a FA-Daa according to this invention is soluble at pH values found in GI-tract, particularly in the 2.0 to 8.0 range. In one aspect a FA-Daa according to this invention is soluble at pH values from pH 2.0 to 8.0. In one aspect a FA-Daa according to this invention is soluble at pH values from pH 2.0 to 4.0. In one aspect a FA-Daa according to this invention is soluble at pH values from pH 3.0 to 8.0. In one aspect a FA-Daa according to this invention is soluble at pH values from pH 4.0 to 8.0. In one aspect a FA-Daa according to this invention is soluble at pH values from pH 5.0 to 8.0. In one aspect a FA-Daa according to this invention is soluble at pH values from pH 6.0 to 8.0. In one aspect a FA-Daa according to this invention is soluble at pH values from pH 3.0 to 4.0. In one aspect a FA-Daa according to this invention is soluble at pH values from pH 4.0 to 5.0. In one aspect a FA-Daa according to this invention is soluble at pH values from pH 5.0 to 6.0. In one aspect a FA-Daa according to this invention is soluble at pH values from pH 6.0 to 7.0. In one aspect a FA-Daa according to this invention is soluble at pH values from pH 7.0 to 8.0.

In one aspect a FA-Daa according to this invention is soluble at intestinal pH values, particularly in the 5.5 to 8.0 range. In one aspect a FA-Daa according to this invention is soluble at intestinal pH values from 5.5 to 8.0. In one aspect a FA-Daa according to this invention is soluble at intestinal pH values from 6.5 to 8.0. In one aspect a FA-Daa according to this invention is soluble at intestinal pH values from 7.5 to 8.0. In one aspect a FA-Daa according to this invention is soluble at intestinal pH values, particularly in the 6.5 to 7.0 range.

In one aspect a FA-Daa according to this invention has a solubility of at least 5 mg/mL. In one aspect a FA-Daa according to this invention has a solubility of at least 10 mg/mL. In one aspect a FA-Daa according to this invention has a solubility of at least 20 mg/mL. In one aspect a FA-Daa according to this invention has a solubility of at least 30 mg/mL. In one aspect a FA-Daa according to this invention has a solubility of at least 40 mg/mL. In one aspect a FA-Daa according to this invention has a solubility of at least 50 mg/mL. In one aspect a FA-Daa according to this invention has a solubility of at least 60 mg/mL. In one aspect a FA-Daa according to this invention has a solubility of at least 70 mg/mL. In one aspect a FA-Daa according to this invention has a solubility of at least 80 mg/mL. In one aspect a FA-Daa according to this invention has a solubility of at least 90 mg/mL. In one aspect a FA-Daa according to this invention has a solubility of at least 100 mg/mL.

In one aspect a FA-Daa according to this invention has a solubility of at least 5 mg/mL in water. In one aspect a FA-Daa according to this invention has a solubility of at least 10 mg/mL in water. In one aspect a FA-Daa according to this invention has a solubility of at least 20 mg/mL in water. In one aspect a FA-Daa according to this invention has a solubility of at least 30 mg/mL in water. In one aspect a FA-Daa according to this invention has a solubility of at least 40 mg/mL in water. In one aspect a FA-Daa according to this invention has a solubility of at least 50 mg/mL in water. In one aspect a FA-Daa according to this invention has a solubility of at least 60 mg/mL in water. In one aspect a FA-Daa according to this invention has a solubility of at least 70 mg/mL in water. In one aspect a FA-Daa according to this invention has a solubility of at least 80 mg/mL in water. In one aspect a FA-Daa according to this invention has a solubility of at least 90 mg/mL in water. In one aspect a FA-Daa according to this invention has a solubility of at least 100 mg/mL in water.

In one aspect a FA-Daa according to this invention has a solubility of at least 5 mg/mL in fasted state simulated intestinal fluid (FASSIF). In one aspect a FA-Daa according to this invention has a solubility of at least 10 mg/mL in FASSIF. In one aspect a FA-Daa according to this invention has a solubility of at least 20 mg/mL in FASSIF. In one aspect a FA-Daa according to this invention has a solubility of at least 30 mg/mL in FASSIF. In one aspect a FA-Daa according to this invention has a solubility of at least 40 mg/mL in FASSIF. In one aspect a FA-Daa according to this invention has a solubility of at least 50 mg/mL in FASSIF. In one aspect a FA-Daa according to this invention has a solubility of at least 60 mg/mL in FASSIF. In one aspect a FA-Daa according to this invention has a solubility of at least 70 mg/mL in FASSIF. In one aspect a FA-Daa according to this invention has a solubility of at least 80 mg/mL in FASSIF. In one aspect a FA-Daa according to this invention has a solubility of at least 90 mg/mL in FASSIF. In one aspect a FA-Daa according to this invention has a solubility of at least 100 mg/mL in FASSIF.

In one aspect a FA-Daa according to this invention has a solubility of at least 5 mg/mL in fed state simulated intestinal fluid (FESSIF). In one aspect a FA-Daa according to this invention has a solubility of at least 10 mg/mL in FESSIF. In one aspect a FA-Daa according to this invention has a solubility of at least 20 mg/mL in FESSIF. In one aspect a FA-Daa according to this invention has a solubility of at least 30 mg/mL in FESSIF. In one aspect a FA-Daa according to this invention has a solubility of at least 40 mg/mL in FESSIF. In one aspect a FA-Daa according to this invention has a solubility of at least 50 mg/mL in FESSIF. In one aspect a FA-Daa according to this invention has a solubility of at least 60 mg/mL in FESSIF. In one aspect a FA-Daa according to this invention has a solubility of at least 70 mg/mL in FESSIF. In one aspect a FA-Daa according to this invention has a solubility of at least 80 mg/mL in FESSIF. In one aspect a FA-Daa according to this invention has a solubility of at least 90 mg/mL in FESSIF. In one aspect a FA-Daa according to this invention has a solubility of at least 100 mg/mL in FESSIF.

A FA-Daa according to the present invention may be represented by the general formula A-Xy, wherein A is an amino acid residue, based on a D-amino acid and Xy is a fatty acid attached by acylation to A's alpha amino group and y is the number of carbon atoms in said fatty acid.

A FA-Daa according to the present invention may be represented by the general formula A-Xy, wherein A is an non-polar uncharged amino acid residue, based on a D-amino acid and Xy is a fatty acid attached by acylation to A's alpha amino group and y is the number of carbon atoms in said fatty acid.

A FA-Daa according to the present invention may be represented by the general formula A-Xy, wherein A is an acidic amino acid residue, based on a D-amino acid and Xy is a fatty acid attached by acylation to A's alpha amino group and y is the number of carbon atoms in said fatty acid.

A FA-Daa according to the present invention may be represented by the general formula A-Xy, wherein A is an non-polar uncharged amino acid residue, based on a D-amino acid and Xy is a fatty acid attached by acylation to A's alpha amino group and y is the number of carbon atoms in said fatty acid, wherein y is 12, 14, 16 or 18.

A FA-Daa according to the present invention may be represented by the general formula A-Xy, wherein A is an acidic amino acid residue, based on a D-amino acid and Xy is a fatty acid attached by acylation to A's alpha amino group and y is the number of carbon atoms in said fatty acid, wherein y is 16 or 18. A FA-Daa according to the present invention may be represented by the general formula A-Xy, wherein A is an acidic amino acid residue, based on a D-amino acid and Xy is a fatty acid attached by acylation to A's alpha amino group and y is the number of carbon atoms in said fatty acid, wherein y is 16. A FA-Daa according to the present invention may be represented by the general formula A-Xy, wherein A is an acidic amino acid residue, based on a D-amino acid and Xy is a fatty acid attached by acylation to A's alpha amino group and y is the number of carbon atoms in said fatty acid, wherein y is 18.

A FA-Daa according to the present invention may be represented by the general formula A-Xy, wherein A is an non-polar uncharged amino acid residue, based on a D-amino acid and Xy is a fatty acid attached by acylation to A's alpha amino group and y is the number of carbon atoms in said fatty acid, wherein y is 12 or 14. A FA-Daa according to the present invention may be represented by the general formula A-Xy, wherein A is an non-polar uncharged amino acid residue, based on a D-amino acid and Xy is a fatty acid attached by acylation to A's alpha amino group and y is the number of carbon atoms in said fatty acid, wherein y is 12. A FA-Daa according to the present invention may be represented by the general formula A-Xy, wherein A is an non-polar uncharged amino acid residue, based on a D-amino acid and Xy is a fatty acid attached by acylation to A's alpha amino group and y is the number of carbon atoms in said fatty acid, wherein y is 14.

A FA-Daa according to the present invention may be represented by the general formula A-Xy, wherein A is an non-polar uncharged amino acid residue, based on a D-amino acid and Xy is a fatty acid attached by acylation to A's alpha amino group and y is the number of carbon atoms in said fatty acid, wherein y is 16 or 18.

TABLE 1

| | Number of carbon atoms in the FA-chain | | | | |
|---|---|---|---|---|---|
| D-amino acid | 10 | 12 | 14 | 16 | 18 |

acidic

| | | |
|---|---|---|
| Aspartic acid | X | X |
| (Asp, D) | | |
| Glutamic acid | X | X |
| (Glu, E) | | |

nonpolar uncharged

| | | | | |
|---|---|---|---|---|
| Alanine | X | X | X | X |
| (Ala, A) | | | | |
| Isoleucine | X | X | X | X |
| (Ile, I) | | | | |
| Leucine | X | X | X | X |
| (Leu, L) | | | | |
| Proline | X | X | X | X |
| (Pro, P) | | | | |
| Valine | X | X | X | X |
| (Val, V) | | | | |

A FA-Daa according to this invention may be represented by the general formula A-Xy, wherein A is an amino acid according to table 1 and Xy is a fatty acid side chain attached by acylation to A's alpha amino group and y represents the number of carbon atoms in said fatty acid side chain.

A FA-Daa according to this invention may be represented by the general formula A-Xy, wherein A is an amino acid according to table 1 and Xy is a fatty acid side chain attached by acylation to A's alpha amino group and y is a number of carbon atoms according to table 1.

A FA-Daa according to this invention may be represented by the general formula A-Xy, wherein A is an amino acid according to table 1 and Xy is a fatty acid side chain attached by acylation to A's alpha amino, wherein the A-Xy combinations according to table 1 represent individual aspects of the present invention.

TABLE 1A

| | Number of carbon atoms in the FA-chain | | | | |
|---|---|---|---|---|---|
| D-amino acid | 10 | 12 | 14 | 16 | 18 |

acidic

| | | |
|---|---|---|
| Aspartic acid | X | X |
| (Asp, D) | | |
| Glutamic acid | X | X |
| (Glu, E) | | |

nonpolar uncharged

| | | | |
|---|---|---|---|
| Alanine | X | X | X |
| (Ala, A) | | | |
| Isoleucine | X | X | X |
| (Ile, I) | | | |
| Leucine | X | X | X |
| (Leu, L) | | | |
| Proline | X | X | X |
| (Pro, P) | | | |
| Valine | X | X | X |
| (Val, V) | | | |

A FA-Daa according to this invention may be represented by the general formula A-Xy, wherein A is an amino acid according to table 1 and Xy is a fatty acid side chain attached by acylation to A's alpha amino group and y represents the number of carbon atoms in said fatty acid side chain.

A FA-Daa according to this invention may be represented by the general formula A-Xy, wherein A is an amino acid according to table 1 and Xy is a fatty acid side chain attached by acylation to A's alpha amino group and y is a number of carbon atoms according to table 1A.

A FA-Daa according to this invention may be represented by the general formula A-Xy, wherein A is an amino acid according to table 1 and Xy is a fatty acid side chain attached by acylation to A's alpha amino, wherein the A-Xy combinations according to table 1A represent individual aspects of the present invention.

Thus, in one aspect a FA-Daa according to the present invention A in the general formula A is D-Aspartic acid and Xy is a fatty acid side chain attached by acylation to D-Aspartic acid's alpha amino group, wherein y is 16 or 18.

In one aspect a FA-Daa according to the present invention A in the general formula A is D-Glutamic acid and Xy is a fatty acid side chain attached by acylation to D-Glutamic acid's alpha amino group, wherein y is 16 or 18.

In one aspect a FA-Daa according to the present invention A in the general formula A is D-Alanine and Xy is a fatty acid side chain attached by acylation to D-Alanine's alpha amino group, wherein y is 12, 14, 16 or 18. In one aspect a FA-Daa according to the present invention A in the general formula A is D-Isoleucine and Xy is a fatty acid side chain attached by acylation to D-Isoleucine's alpha amino group, wherein y is 12, 14, 16 or 18. In one aspect a FA-Daa according to the present invention A in the general formula A is D-Leucine and Xy is a fatty acid side chain attached by acylation to D-Leucine's alpha amino group, wherein y is 12, 14, 16 or 18. In one aspect a FA-Daa according to the present invention A in the general formula A is D-Proline and Xy is a fatty acid side chain attached by acylation to D-Proline's alpha amino group, wherein y is 12, 14, 16 or 18. In one aspect a FA-Daa according to the present invention A in the general formula A is D-Valine and Xy is a fatty acid side chain attached by acylation to D-Valine's alpha amino group, wherein y is 12, 14, 16 or 18.

In one aspect a FA-Daa according to the present invention A in the general formula A is D-Alanine and Xy is a fatty acid side chain attached by acylation to D-Alanine's alpha amino group, wherein y is 16 or 18. In one aspect a FA-Daa according to the present invention A in the general formula A is D-Alanine and Xy is a fatty acid side chain attached by acylation to D-Alanine's alpha amino group, wherein y is 16. In one aspect a FA-Daa according to the present invention A in the general formula A is D-Alanine and Xy is a fatty acid side chain attached by acylation to D-Alanine's alpha amino group, wherein y is 18.

In one aspect a FA-Daa according to the present invention A in the general formula A is D-Isoleucine and Xy is a fatty acid side chain attached by acylation to D-Isoleucine's alpha amino group, wherein y is 16 or 18. In one aspect a FA-Daa according to the present invention A in the general formula A is D-Isoleucine and Xy is a fatty acid side chain attached by acylation to D-Isoleucine's alpha amino group, wherein y is 16. In one aspect a FA-Daa according to the present invention A in the general formula A is D-Isoleucine and Xy is a fatty acid side chain attached by acylation to D-Isoleucine's alpha amino group, wherein y is 18.

In one aspect a FA-Daa according to the present invention A in the general formula A is D-Leucine and Xy is a fatty acid side chain attached by acylation to D-Leucine's alpha amino group, wherein y is 16 or 18. In one aspect a FA-Daa according to the present invention A in the general formula A is D-Leucine and Xy is a fatty acid side chain attached by acylation to D-Leucine's alpha amino group, wherein y is 16. In one aspect a FA-Daa according to the present invention A in the general formula A is D-Leucine and Xy is a fatty acid side chain attached by acylation to D-Leucine's alpha amino group, wherein y is 18.

In one aspect a FA-Daa according to the present invention A in the general formula A is D-Proline and Xy is a fatty acid side chain attached by acylation to D-Proline's alpha amino group, wherein y is 16 or 18. In one aspect a FA-Daa according to the present invention A in the general formula A is D-Proline and Xy is a fatty acid side chain attached by acylation to D-Proline's alpha amino group, wherein y is 16. In one aspect a FA-Daa according to the present invention A in the general formula A is D-Proline and Xy is a fatty acid side chain attached by acylation to D-Proline's alpha amino group, wherein y is 18.

In one aspect a FA-Daa according to the present invention A in the general formula A is D-Valine and Xy is a fatty acid side chain attached by acylation to D-Valine's alpha amino group, wherein y is 16 or 18. In one aspect a FA-Daa according to the present invention A in the general formula A is D-Valine and Xy is a fatty acid side chain attached by acylation to D-Valine's alpha amino group, wherein y is 16. In one aspect a FA-Daa according to the present invention A in the general formula A is D-Valine and Xy is a fatty acid side chain attached by acylation to D-Valine's alpha amino group, wherein y is 18.

TABLE 2

| | Number of carbon atoms in the FA-chain | | | | |
|---|---|---|---|---|---|
| D-amino acid | 10 | 12 | 14 | 16 | 18 |

acidic

| | | |
|---|---|---|
| Aspartic acid | X | X |
| (Asp, D) | | |
| Glutamic acid | X | X |
| (Glu, E) | | |

nonpolar uncharged

| | | |
|---|---|---|
| Alanine | X | X |
| (Ala, A) | | |
| Isoleucine | X | X |
| (Ile, I) | | |
| Leucine | X | X |
| (Leu, L) | | |
| Proline | X | X |
| (Pro, P) | | |
| Valine | X | X |
| (Val, V) | | |

A FA-Daa according to this invention may be represented by the general formula A-Xy, wherein A is an amino acid according to table 2 and Xy is a fatty acid side chain attached by acylation to A's alpha amino group and y represents the number of carbon atoms in said fatty acid side chain.

A FA-Daa according to this invention may be represented by the general formula A-Xy, wherein A is an amino acid according to table 2 and Xy is a fatty acid side chain attached by acylation to A's alpha amino group and y is a number of carbon atoms according to table 2.

A FA-Daa according to this invention may be represented by the general formula A-Xy, wherein A is an amino acid according to table 2 and Xy is a fatty acid side chain attached by acylation to A's alpha amino, wherein the A-Xy combinations according to table 1 represent individual aspects of the present invention.

A FA-Daa according to this invention may be represented by the general formula A-Xy, wherein A is an amino acid according to table 2 and Xy is a fatty acid side chain attached by acylation to A's alpha amino, wherein the A-Xy combinations according to table 1A represent individual aspects of the present invention.

Thus, in one aspect a FA-Daa according to the present invention A in the general formula A is D-Aspartic acid and Xy is a fatty acid side chain attached by acylation to D-Aspartic acid's alpha amino group, wherein y is 16. Thus, in one aspect a FA-Daa according to the present invention A in the general formula A is D-Aspartic acid and Xy is a fatty acid side chain attached by acylation to D-Aspartic acid's alpha amino group, wherein y is 18.

In one aspect a FA-Daa according to the present invention A in the general formula A is D-Glutamic acid and Xy is a fatty acid side chain attached by acylation to D-Glutamic acid's alpha amino group, wherein y is 16. In one aspect a FA-Daa according to the present invention A in the general formula A is D-Glutamic acid and Xy is a fatty acid side chain attached by acylation to D-Glutamic acid's alpha amino group, wherein y is 18.

In one aspect a FA-Daa according to the present invention A in the general formula A is D-Alanine and Xy is a fatty acid side chain attached by acylation to D-Alanine's alpha amino group, wherein y is 12 or 14. In one aspect a FA-Daa according to the present invention A in the general formula A is D-Alanine and Xy is a fatty acid side chain attached by acylation to D-Alanine's alpha amino group, wherein y is 12. In one aspect a FA-Daa according to the present invention A in the general formula A is D-Alanine and Xy is a fatty acid side chain attached by acylation to D-Alanine's alpha amino group, wherein y is 14.

In one aspect a FA-Daa according to the present invention A in the general formula A is D-Isoleucine and Xy is a fatty acid side chain attached by acylation to D-Isoleucine's alpha amino group, wherein y is 12 or 14. In one aspect a FA-Daa according to the present invention A in the general formula A is D-Isoleucine and Xy is a fatty acid side chain attached by acylation to D-Isoleucine's alpha amino group, wherein y is 12. In one aspect a FA-Daa according to the present invention A in the general formula A is D-Isoleucine and Xy is a fatty acid side chain attached by acylation to D-Isoleucine's alpha amino group, wherein y is 14.

In one aspect a FA-Daa according to the present invention A in the general formula A is D-Leucine and Xy is a fatty acid side chain attached by acylation to D-Leucine's alpha amino group, wherein y is 12 or 14. In one aspect a FA-Daa according to the present invention A in the general formula A is D-Leucine and Xy is a fatty acid side chain attached by acylation to D-Leucine's alpha amino group, wherein y is 12. In one aspect a FA-Daa according to the present invention A in the general formula A is D-Leucine and Xy is a fatty acid side chain attached by acylation to D-Leucine's alpha amino group, wherein y is 14.

In one aspect a FA-Daa according to the present invention A in the general formula A is D-Proline and Xy is a fatty acid side chain attached by acylation to D-Proline's alpha amino group, wherein y is 12 or 14. In one aspect a FA-Daa according to the present invention A in the general formula A is D-Proline and Xy is a fatty acid side chain attached by acylation to D-Proline's alpha amino group, wherein y is 12. In one aspect a FA-Daa according to the present invention A in the general formula A is D-Proline and Xy is a fatty acid side chain attached by acylation to D-Proline's alpha amino group, wherein y is 14.

In one aspect a FA-Daa according to the present invention A in the general formula A is D-Valine and Xy is a fatty acid side chain attached by acylation to D-Valine's alpha amino group, wherein y is 12 or 14. In one aspect a FA-Daa according to the present invention A in the general formula A is D-Valine and Xy is a fatty acid side chain attached by acylation to D-Valine's alpha amino group, wherein y is 12. In one aspect a FA-Daa according to the present invention A in the general formula A is D-Valine and Xy is a fatty acid side chain attached by acylation to D-Valine's alpha amino group, wherein y is 14.

A FA-Daa according to the present invention may be represented by the general formula;

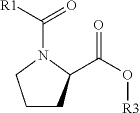

wherein R1 is a hydrocarbon chain comprising 11 to 17 carbon atoms and R3 is either H, or absent.

A FA-Daa according to the present invention may be represented by the general formula;

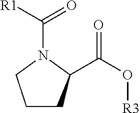

wherein R1 is a hydrocarbon chain comprising 13 to 17 carbon atoms and R3 is either H, or absent.

A FA-Daa according to the present invention may be represented by the general formula;

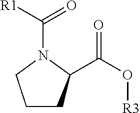

wherein R1 is a hydrocarbon chain comprising 15 to 17 carbon atoms and R3 is either H, or absent.

A FA-Daa according to the present invention may be represented by the general formula;

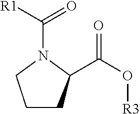

wherein R1 is a hydrocarbon chain comprising 11 to 15 carbon atoms and R3 is either H, or absent.

A FA-Daa according to the present invention may be represented by the general formula;

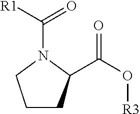

wherein R1 is a hydrocarbon chain comprising 17 carbon atoms and R3 is either H, or absent.

A FA-Daa according to the present invention may be represented by the general formula;

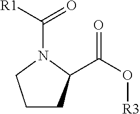

wherein R1 is a hydrocarbon chain comprising 15 carbon atoms and R3 is either H, or absent.

A FA-Daa according to the present invention may be represented by the general formula;

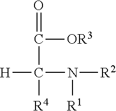

wherein R1 is a fatty acid chain comprising 12 to 18 carbons, R2 is either H (i.e. hydrogen) or CH₃ (i.e. methyl group), R3 is either H, or absent, and R4 is a amino acid side chain of a non-polar uncharged amino acid, selected from the group consisting of: Alanine, Isoleucine, Leucine, Proline and Valine, wherein the stereo configuration of the chiral carbon atom in the amino acid moiety is in the D-configuration, wherein, then R1 comprises 12, 18, 16 or 18 carbon atoms.

In one aspect a FA-Daa according to the present invention may be represented by the general formula:

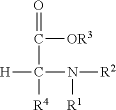

wherein R1 is a fatty acid chain comprising 12 to 18 carbon atoms, R2 is either H (i.e. hydrogen) or CH₃ (i.e. methyl group), R3 is either H, or a potassium (K+) or sodium (Na+) salt thereof, and R4 is a amino acid side chain of a non-polar uncharged amino acid, wherein the stereo configuration of the chiral carbon atom in the amino acid moiety is D.

A FA-Daa according to the present invention may be represented by the general formula;

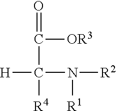

wherein R1 is a fatty acid chain comprising 16 to 18 carbons, R2 is either H (i.e. hydrogen) or CH₃ (i.e. methyl group), R3 is either H, or absent, and R4 is a amino acid side chain of a non-polar uncharged amino acid, wherein the stereo configuration of the chiral carbon atom in the amino acid moiety is in the D-configuration.

In one aspect a FA-Daa according to the present invention may be represented by the general formula:

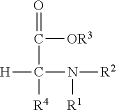

wherein R1 is a fatty acid chain comprising 16 to 18 carbon atoms, R2 is either H (i.e. hydrogen) or CH₃ (i.e. methyl group), R3 is either H, or a potassium (K+) or sodium (Na+) salt thereof, and R4 is a amino acid side chain of a non-polar uncharged amino acid, wherein the stereo configuration of the chiral carbon atom in the amino acid moiety is D.

A FA-Daa according to the present invention may be represented by the general formula;

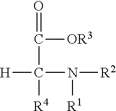

wherein R1 is a fatty acid chain comprising 12 to 14 carbons, R2 is either H (i.e. hydrogen) or CH₃ (i.e. methyl group), R3 is either H, or absent, and R4 is a amino acid side chain of a non-polar uncharged amino acid, wherein the stereo configuration of the chiral carbon atom in the amino acid moiety is in the D-configuration.

In one aspect a FA-Daa according to the present invention may be represented by the general formula:

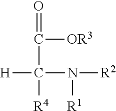

wherein R1 is a fatty acid chain comprising 12 to 14 carbon atoms, R2 is either H (i.e. hydrogen) or CH₃ (i.e. methyl group), R3 is either H, or a potassium (K+) or sodium (Na+) salt thereof, and R4 is a amino acid side chain of a non-polar uncharged amino acid, wherein the stereo configuration of the chiral carbon atom in the amino acid moiety is D.

A FA-Daa according to the present invention may be represented by the general formula;

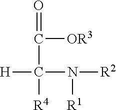

wherein R1 is a fatty acid chain comprising 12 carbons, R2 is either H (i.e. hydrogen) or CH₃ (i.e. methyl group), R3 is either H, or absent, and R4 is a amino acid side chain of a non-polar uncharged amino acid, wherein the stereo configuration of the chiral carbon atom in the amino acid moiety is in the D-configuration.

In one aspect a FA-Daa according to the present invention may be represented by the general formula:

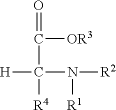

wherein R1 is a fatty acid chain comprising 12 carbon atoms, R2 is either H (i.e. hydrogen) or CH₃ (i.e. methyl group), R3 is either H, or a potassium (K+) or sodium (Na+) salt thereof, and R4 is a amino acid side chain of a non-polar uncharged amino acid, wherein the stereo configuration of the chiral carbon atom in the amino acid moiety is D, wherein R1 comprises 12 carbon atoms.

A FA-Daa according to the present invention may be represented by the general formula;

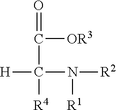

wherein R1 is a fatty acid chain comprising 14 carbons, R2 is either H (i.e. hydrogen) or CH₃ (i.e. methyl group), R3 is either H, or absent, and R4 is a amino acid side chain of a non-polar uncharged amino acid, wherein the stereo configuration of the chiral carbon atom in the amino acid moiety is in the D-configuration.

In one aspect a FA-Daa according to the present invention may be represented by the general formula:

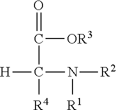

wherein R1 is a fatty acid chain comprising 14 carbon atoms, R2 is either H (i.e. hydrogen) or CH₃ (i.e. methyl group), R3 is either H, or a potassium (K+) or sodium (Na+) salt thereof, and R4 is a amino acid side chain of a non-polar uncharged amino acid, wherein the stereo configuration of the chiral carbon atom in the amino acid moiety is D.

A FA-Daa according to the present invention may be represented by the general formula;

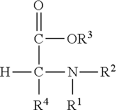

wherein R1 is a fatty acid chain comprising 16 carbons, R2 is either H (i.e. hydrogen) or CH₃ (i.e. methyl group), R3 is either H, or absent, and R4 is a amino acid side chain of a non-polar uncharged amino acid, wherein the stereo configuration of the chiral carbon atom in the amino acid moiety is in the D-configuration.

In one aspect a FA-Daa according to the present invention may be represented by the general formula:

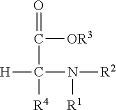

wherein R1 is a fatty acid chain comprising 16 carbon atoms, R2 is either H (i.e. hydrogen) or CH₃ (i.e. methyl group), R3 is either H, or a potassium (K+) or sodium (Na+) salt thereof, and R4 is a amino acid side chain of a non-polar uncharged amino acid, wherein the stereo configuration of the chiral carbon atom in the amino acid moiety is D.

A FA-Daa according to the present invention may be represented by the general formula;

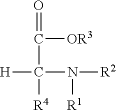

wherein R1 is a fatty acid chain comprising 18 carbons, R2 is either H (i.e. hydrogen) or CH₃ (i.e. methyl group), R3 is either H, or absent, and R4 is a amino acid side chain of a non-polar uncharged amino acid, wherein the stereo configuration of the chiral carbon atom in the amino acid moiety is in the D-configuration.

In one aspect a FA-Daa according to the present invention may be represented by the general formula:

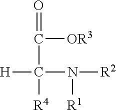

wherein R1 is a fatty acid chain comprising 18 carbon atoms, R2 is either H (i.e. hydrogen) or CH₃ (i.e. methyl group), R3 is either H, or a potassium (K+) or sodium (Na+) salt thereof, and R4 is a amino acid side chain of a non-polar uncharged amino acid, wherein the stereo configuration of the chiral carbon atom in the amino acid moiety is D.

A FA-Daa according to the present invention may be represented by the general formula;

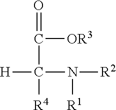

wherein R1 is a fatty acid chain comprising 12 to 18 carbons, R2 is either H (i.e. hydrogen) or CH₃ (i.e. methyl group), R3 is either H, or absent, and R4 is a amino acid side chain of a non-polar uncharged amino acid, selected from the group consisting of: Alanine, Isoleucine, Leucine, Proline and Valine, wherein the stereo configuration of the chiral carbon atom in the amino acid moiety is in the D-configuration, wherein, then R1 comprises 12, 18, 16 or 18 carbon atoms.

In one aspect a FA-Daa according to the present invention may be represented by the general formula:

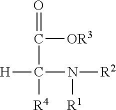

wherein R1 is a fatty acid chain comprising 12 to 18 carbon atoms, R2 is either H (i.e. hydrogen) or CH₃ (i.e. methyl group), R3 is either H, or a potassium (K+) or sodium (Na+) salt thereof, and R4 is a amino acid side chain of a non-polar uncharged amino acid, selected from the group consisting of: Alanine, Isoleucine, Leucine, Proline and Valine, wherein the stereo configuration of the chiral carbon atom in the amino acid moiety is D.

A FA-Daa according to the present invention may be represented by the general formula;

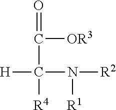

wherein R1 is a fatty acid chain comprising 16 to 18 carbons, R2 is either H (i.e. hydrogen) or CH₃ (i.e. methyl group), R3 is either H, or absent, and R4 is a amino acid side chain of a non-polar uncharged amino acid, selected from the group consisting of: Alanine, Isoleucine, Leucine, Proline and Valine, wherein the stereo configuration of the chiral carbon atom in the amino acid moiety is in the D-configuration.

In one aspect a FA-Daa according to the present invention may be represented by the general formula:

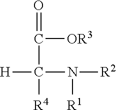

wherein R1 is a fatty acid chain comprising 16 to 18 carbon atoms, R2 is either H (i.e. hydrogen) or CH₃ (i.e. methyl group), R3 is either H, or a potassium (K+) or sodium (Na+) salt thereof, and R4 is a amino acid side chain of a non-polar uncharged amino acid, selected from the group consisting of: Alanine, Isoleucine, Leucine, Proline and Valine, wherein the stereo configuration of the chiral carbon atom in the amino acid moiety is D.

A FA-Daa according to the present invention may be represented by the general formula;

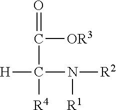

wherein R1 is a fatty acid chain comprising 12 to 14 carbons, R2 is either H (i.e. hydrogen) or CH₃ (i.e. methyl group), R3 is either H, or absent, and R4 is a amino acid side chain of a non-polar uncharged amino acid, selected from the group consisting of: Alanine, Isoleucine, Leucine, Proline and Valine, wherein the stereo configuration of the chiral carbon atom in the amino acid moiety is in the D-configuration.

In one aspect a FA-Daa according to the present invention may be represented by the general formula:

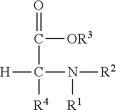

wherein R1 is a fatty acid chain comprising 12 to 14 carbon atoms, R2 is either H (i.e. hydrogen) or CH₃ (i.e. methyl group), R3 is either H, or a potassium (K+) or sodium (Na+) salt thereof, and R4 is a amino acid side chain of a non-polar uncharged amino acid, selected from the group consisting of: Alanine, Isoleucine, Leucine, Proline and Valine, wherein the stereo configuration of the chiral carbon atom in the amino acid moiety is D.

A FA-Daa according to the present invention may be represented by the general formula;

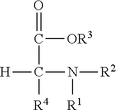

wherein R1 is a fatty acid chain comprising 12 carbons, R2 is either H (i.e. hydrogen) or CH₃ (i.e. methyl group), R3 is either H, or absent, and R4 is a amino acid side chain of a non-polar uncharged amino acid, selected from the group consisting of: Alanine, Isoleucine, Leucine, Proline and Valine, wherein the stereo configuration of the chiral carbon atom in the amino acid moiety is in the D-configuration.

In one aspect a FA-Daa according to the present invention may be represented by the general formula:

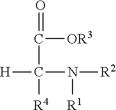

wherein R1 is a fatty acid chain comprising 12 carbon atoms, R2 is either H (i.e. hydrogen) or CH₃ (i.e. methyl group), R3 is either H, or a potassium (K+) or sodium (Na+) salt thereof, and R4 is a amino acid side chain of a non-polar uncharged amino acid, selected from the group consisting of: Alanine, Isoleucine, Leucine, Proline and Valine, wherein the stereo configuration of the chiral carbon atom in the amino acid moiety is D, wherein R1 comprises 12 carbon atoms.

A FA-Daa according to the present invention may be represented by the general formula;

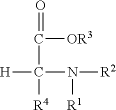

wherein R1 is a fatty acid chain comprising 14 carbons, R2 is either H (i.e. hydrogen) or CH₃ (i.e. methyl group), R3 is either H, or absent, and R4 is a amino acid side chain of a non-polar uncharged amino acid, selected from the group consisting of: Alanine, Isoleucine, Leucine, Proline and Valine, wherein the stereo configuration of the chiral carbon atom in the amino acid moiety is in the D-configuration.

In one aspect a FA-Daa according to the present invention may be represented by the general formula:

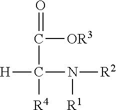

wherein R1 is a fatty acid chain comprising 14 carbon atoms, R2 is either H (i.e. hydrogen) or CH₃ (i.e. methyl group), R3 is either H, or a potassium (K+) or sodium (Na+) salt thereof, and R4 is a amino acid side chain of a non-polar uncharged amino acid, selected from the group consisting of: Alanine, Isoleucine, Leucine, Proline and Valine, wherein the stereo configuration of the chiral carbon atom in the amino acid moiety is D.

A FA-Daa according to the present invention may be represented by the general formula;

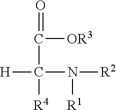

wherein R1 is a fatty acid chain comprising 16 carbons, R2 is either H (i.e. hydrogen) or CH₃ (i.e. methyl group), R3 is either H, or absent, and R4 is a amino acid side chain of a non-polar uncharged amino acid, selected from the group consisting of: Alanine, Isoleucine, Leucine, Proline and Valine, wherein the stereo configuration of the chiral carbon atom in the amino acid moiety is in the D-configuration.

In one aspect a FA-Daa according to the present invention may be represented by the general formula:

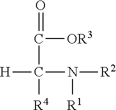

wherein R1 is a fatty acid chain comprising 16 carbon atoms, R2 is either H (i.e. hydrogen) or CH₃ (i.e. methyl group), R3 is either H, or a potassium (K+) or sodium (Na+) salt thereof, and R4 is a amino acid side chain of a non-polar uncharged amino acid, selected from the group consisting of: Alanine, Isoleucine, Leucine, Proline and Valine, wherein the stereo configuration of the chiral carbon atom in the amino acid moiety is D.

A FA-Daa according to the present invention may be represented by the general formula;

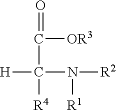

wherein R1 is a fatty acid chain comprising 18 carbons, R2 is either H (i.e. hydrogen) or CH₃ (i.e. methyl group), R3 is either H, or absent, and R4 is a amino acid side chain of a non-polar uncharged amino acid, selected from the group consisting of: Alanine, Isoleucine, Leucine, Proline and Valine, wherein the stereo configuration of the chiral carbon atom in the amino acid moiety is in the D-configuration.

In one aspect a FA-Daa according to the present invention may be represented by the general formula:

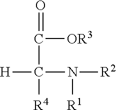

wherein R1 is a fatty acid chain comprising 18 carbon atoms, R2 is either H (i.e. hydrogen) or CH₃ (i.e. methyl group), R3 is either H, or a potassium (K+) or sodium (Na+) salt thereof, and R4 is a amino acid side chain of a non-polar uncharged amino acid, selected from the group consisting of: Alanine, Isoleucine, Leucine, Proline and Valine, wherein the stereo configuration of the chiral carbon atom in the amino acid moiety is D.

A FA-Daa according to the present invention may be represented by the general formula;

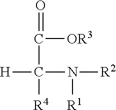

wherein R1 is a fatty acid chain comprising 16 to 18 carbons, R2 is either H (i.e. hydrogen) or CH₃ (i.e. methyl group), R3 is either H, or absent, and R4 is a amino acid side chain of a acidic amino acid, wherein the stereo configuration of the chiral carbon atom in the amino acid moiety is in the D-configuration.

In one aspect a FA-Daa according to the present invention may be represented by the general formula:

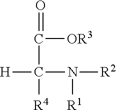

wherein R1 is a fatty acid chain comprising 16 to 18 carbon atoms, R2 is either H (i.e. hydrogen) or CH₃ (i.e. methyl group), R3 is either H, or a potassium (K+) or sodium (Na+) salt thereof, and R4 is a amino acid side chain of a acidic amino acid, wherein the stereo configuration of the chiral carbon atom in the amino acid moiety is D.

A FA-Daa according to the present invention may be represented by the general formula;

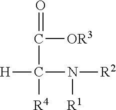

wherein R1 is a fatty acid chain comprising 16 carbons, R2 is either H (i.e. hydrogen) or CH₃ (i.e. methyl group), R3 is either H, or absent, and R4 is a amino acid side chain of a acidic amino acid, wherein the stereo configuration of the chiral carbon atom in the amino acid moiety is in the D-configuration.

In one aspect a FA-Daa according to the present invention may be represented by the general formula:

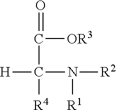

wherein R1 is a fatty acid chain comprising 16 carbon atoms, R2 is either H (i.e. hydrogen) or CH₃ (i.e. methyl group), R3 is either H, or a potassium (K+) or sodium (Na+) salt thereof, and R4 is a amino acid side chain of a acidic amino acid, wherein the stereo configuration of the chiral carbon atom in the amino acid moiety is D.

A FA-Daa according to the present invention may be represented by the general formula;

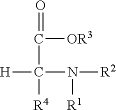

wherein R1 is a fatty acid chain comprising 18 carbons, R2 is either H (i.e. hydrogen) or CH₃ (i.e. methyl group), R3 is either H, or absent, and R4 is a amino acid side chain of a acidic amino acid, wherein the stereo configuration of the chiral carbon atom in the amino acid moiety is in the D-configuration.

In one aspect a FA-Daa according to the present invention may be represented by the general formula:

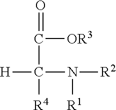

wherein R1 is a fatty acid chain comprising 18 carbon atoms, R2 is either H (i.e. hydrogen) or CH₃ (i.e. methyl group), R3 is either H, or a potassium (K+) or sodium (Na+) salt thereof, and R4 is a amino acid side chain of a acidic amino acid, wherein the stereo configuration of the chiral carbon atom in the amino acid moiety is D.

A FA-Daa according to the present invention may be represented by the general formula;

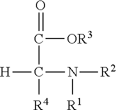

wherein R1 is a fatty acid chain comprising 16 to 18 carbons, R2 is either H (i.e. hydrogen) or CH₃ (i.e. methyl group), R3 is either H, or absent, and R4 is a amino acid side chain of a acidic amino acid selected from the roup consisting of Aspartic acid and Glutamic acid, wherein the stereo configuration of the chiral carbon atom in the amino acid moiety is in the D-configuration.

In one aspect a FA-Daa according to the present invention may be represented by the general formula:

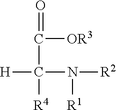

wherein R1 is a fatty acid chain comprising 16 to 18 carbon atoms, R2 is either H (i.e. hydrogen) or CH₃ (i.e. methyl group), R3 is either H, or a potassium (K+) or sodium (Na+) salt thereof, and R4 is a amino acid side chain of a acidic amino acid selected from the roup consisting of Aspartic acid and Glutamic acid, wherein the stereo configuration of the chiral carbon atom in the amino acid moiety is D.

A FA-Daa according to the present invention may be represented by the general formula;

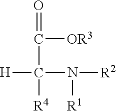

wherein R1 is a fatty acid chain comprising 16 carbons, R2 is either H (i.e. hydrogen) or CH₃ (i.e. methyl group), R3 is either H, or absent, and R4 is a amino acid side chain of a acidic amino acid selected from the roup consisting of Aspartic acid and Glutamic acid, wherein the stereo configuration of the chiral carbon atom in the amino acid moiety is in the D-configuration.

In one aspect a FA-Daa according to the present invention may be represented by the general formula:

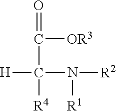

wherein R1 is a fatty acid chain comprising 16 carbon atoms, R2 is either H (i.e. hydrogen) or CH₃ (i.e. methyl group), R3 is either H, or a potassium (K+) or sodium (Na+) salt thereof, and R4 is a amino acid side chain of a acidic amino acid selected from the roup consisting of Aspartic acid and Glutamic acid, wherein the stereo configuration of the chiral carbon atom in the amino acid moiety is D.

A FA-Daa according to the present invention may be represented by the general formula;

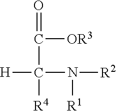

wherein R1 is a fatty acid chain comprising 18 carbons, R2 is either H (i.e. hydrogen) or CH₃ (i.e. methyl group), R3 is either H, or absent, and R4 is a amino acid side chain of a acidic amino acid selected from the roup consisting of Aspartic acid and Glutamic acid, wherein the stereo configuration of the chiral carbon atom in the amino acid moiety is in the D-configuration.

In one aspect a FA-Daa according to the present invention may be represented by the general formula:

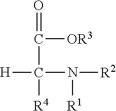

wherein R1 is a fatty acid chain comprising 18 carbon atoms, R2 is either H (i.e. hydrogen) or CH₃ (i.e. methyl group), R3 is either H, or a potassium (K+) or sodium (Na+) salt thereof, and R4 is a amino acid side chain of a acidic amino acid selected from the roup consisting of Aspartic acid and Glutamic acid, wherein the stereo configuration of the chiral carbon atom in the amino acid moiety is D.

In one aspect a FA-Daa according to this invention may be chosen from the group consisting of formula (d) wherein R1 is a hydrocarbon chain comprising 11 to 17 carbons, R2 is either H (i.e. hydrogen) or CH₃ (i.e. methyl group), and R3 is either H, or a salt thereof. In one aspect a FA-Daa according to this invention may be chosen from the group consisting of formula (d) wherein R1 is a hydrocarbon chain comprising 11 carbons, R2 is either H (i.e. hydrogen) or CH₃ (i.e. methyl group), and R3 is either H, or a salt thereof. In one aspect a FA-Daa according to this invention may be chosen from the group consisting of formula (d) wherein R1 is a hydrocarbon chain comprising 13 carbons, R2 is either H (i.e. hydrogen) or CH₃ (i.e. methyl group), and R3 is either H, or a salt thereof. In one aspect a FA-Daa according to this invention may be chosen from the group consisting of formula (d) wherein R1 is a hydrocarbon chain comprising 13 carbons, R2 is either H (i.e. hydrogen) or CH₃ (i.e. methyl group), and R3 is either H, or a salt thereof. In one aspect a FA-Daa according to this invention may be chosen from the group consisting of formula (d) wherein R1 is a hydrocarbon chain comprising 15 carbons, R2 is either H (i.e. hydrogen) or CH₃ (i.e. methyl group), and R3 is either H, or a salt thereof. In one aspect a FA-Daa according to this invention may be chosen from the group consisting of formula (d) wherein R1 is a hydrocarbon chain comprising 17 carbons, R2 is either H (i.e. hydrogen) or CH₃ (i.e. methyl group), and R3 is either H, or a salt thereof.

In one aspect a FA-Daa according to this invention may be chosen from the group consisting of formula (d) wherein R1 is a hydrocarbon chain comprising 11 to 17 carbon atoms, R2 is either H (i.e. hydrogen) or CH₃ (i.e. methyl group), and R3 is either H, or a sodium (Na+) or potassium (K+) salt thereof. In one aspect a FA-Daa according to this invention may be chosen from the group consisting of formula (d) wherein R1 is a hydrocarbon chain comprising 11 carbon atoms, R2 is either H (i.e. hydrogen) or CH₃ (i.e. methyl group), and R3 is either H, or a sodium (Na+) or potassium (K+) salt thereof. In one aspect a FA-Daa according to this invention may be chosen from the group consisting of formula (d) wherein R1 is a hydrocarbon chain comprising 13 carbon atoms, R2 is either H (i.e. hydrogen) or CH₃ (i.e. methyl group), and R3 is either H, or a sodium (Na+) or potassium (K+) salt thereof. In one aspect a FA-Daa according to this invention may be chosen from the group consisting of formula (d) wherein R1 is a hydrocarbon chain comprising 15 carbon atoms, R2 is either H (i.e. hydrogen) or CH₃ (i.e. methyl group), and R3 is either H, or a sodium (Na+) or potassium (K+) salt thereof. In one aspect a FA-Daa according to this invention may be chosen from the group consisting of formula (d) wherein R1 is a hydrocarbon chain comprising 17 carbon atoms, R2 is either H (i.e. hydrogen) or CH₃ (i.e. methyl group), and R3 is either H, or a sodium (Na+) or potassium (K+) salt thereof.

In one aspect a FA-Daa according to this invention may be chosen from the group consisting of formula (d) wherein R1 is a hydrocarbon chain comprising 13 to 17 carbons, R2 is either H (i.e. hydrogen) or CH₃ (i.e. methyl group), and R3 is either H, or a salt thereof.

In one aspect a FA-Daa according to this invention may be chosen from the group consisting of formula (d) wherein R1 is a hydrocarbon chain comprising 13 to 17 carbon atoms, R2 is either H (i.e. hydrogen) or CH₃ (i.e. methyl group), and R3 is either H, or a sodium (Na+) or potassium (K+) salt thereof.

In one aspect a FA-Daa according to this invention may be chosen from the group consisting of (d) wherein R1 is a hydrocarbon chain comprising 15 to 17 carbons, R2 is either H (i.e. hydrogen) or CH₃ (i.e. methyl group), and R3 is either H, or a salt thereof.

In one aspect a FA-Daa according to this invention may be chosen from the group consisting of (d) wherein R1 is a hydrocarbon chain comprising 15 to 17 carbon atoms, R2 is either H (i.e. hydrogen) or CH₃ (i.e. methyl group), and R3 is either H, or a sodium (Na+) or potassium (K+) salt thereof.

A FA-Daa according to the present invention may be represented by the general formula;

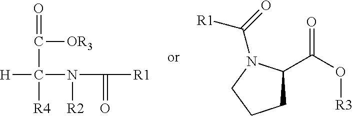

wherein R1 is a hydrocarbon chain comprising 11 to 17 carbons, R2 is either H (i.e. hydrogen) or CH₃ (i.e. methyl group), R3 is either H, or absent, and R4 is an amino acid side chain, wherein the stereo configuration of the chiral carbon atom in the amino acid moiety is in the D-configuration, with the proviso that when R4 is from a non-polar uncharged amino acid, then R1 comprises 11, 13, 15 or 17 carbon atoms and when R4 is from an acidic amino acid, then R1 comprises 15 or 17 carbon atoms.

In one aspect a FA-Daa according to the present invention may be represented by the general formula:

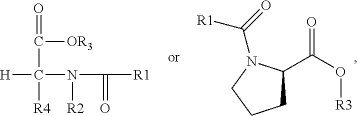

wherein R1 is a hydrocarbon chain comprising 11 to 17 carbon atoms, R2 is either H (i.e. hydrogen) or CH₃ (i.e. methyl group), R3 is either H, or a potassium (K+) or sodium (Na+) salt thereof, and R4 is a amino acid side chain, wherein the stereo configuration of the chiral carbon atom in the amino acid moiety is D, with the proviso that when R4 is from a non-polar uncharged amino acid, then R1 comprises 11, 13, 15 or 17 carbon atoms and when R4 is from an acidic amino acid, then R1 comprises 15 or 17 carbon atoms.

A FA-Daa according to the present invention may be represented by the general formula;

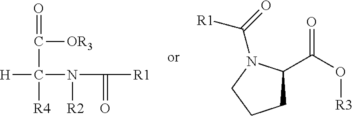

wherein R1 is a hydrocarbon chain comprising 15 to 17 carbons, R2 is either H (i.e. hydrogen) or CH₃ (i.e. methyl group), R3 is either H, or absent, and R4 is an amino acid side chain, wherein the stereo configuration of the chiral carbon atom in the amino acid moiety is in the D-configuration, with the proviso that when R4 is from a non-polar uncharged amino acid, then R1 comprises 15 carbon atoms and when R4 is from an acidic amino acid, then R1 comprises 15 carbon atoms.

In one aspect a FA-Daa according to the present invention may be represented by the general formula:

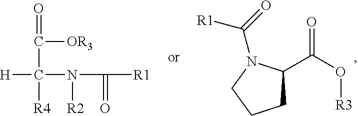

wherein R1 is a hydrocarbon chain comprising 13 to 15 carbon atoms, R2 is either H (i.e. hydrogen) or CH₃ (i.e. methyl group), R3 is either H, or a potassium (K+) or sodium (Na+) salt thereof, and R4 is a amino acid side chain, wherein the stereo configuration of the chiral carbon atom in the amino acid moiety is D, with the proviso that when R4 is from a non-polar uncharged amino acid, then R1 comprises 15 carbon atoms and when R4 is from an acidic amino acid, then R1 comprises 15 carbon atoms.

A FA-Daa according to the present invention may be represented by the general formula;

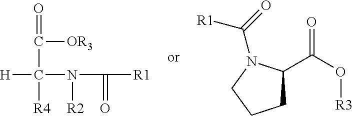

wherein R1 is a hydrocarbon chain comprising 13 to 15 carbons, R2 is either H (i.e. hydrogen) or CH₃ (i.e. methyl group), R3 is either H, or absent, and R4 is an amino acid side chain, wherein the stereo configuration of the chiral carbon atom in the amino acid moiety is in the D-configuration, with the proviso that when R4 is from a non-polar uncharged amino acid, then R1 comprises 13 carbon atoms and when R4 is from an acidic amino acid, then R1 comprises 13 carbon atoms.

In one aspect a FA-Daa according to the present invention may be represented by the general formula:

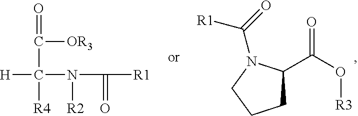

wherein R1 is a hydrocarbon chain comprising 13 to 15 carbon atoms, R2 is either H (i.e. hydrogen) or CH₃ (i.e. methyl group), R3 is either H, or a potassium (K+) or sodium (Na+) salt thereof, and R4 is a amino acid side chain, wherein the stereo configuration of the chiral carbon atom in the amino acid moiety is D, with the proviso that when R4 is from a non-polar uncharged amino acid, then R1 comprises 13 carbon atoms and when R4 is from an acidic amino acid, then R1 comprises 13 carbon atoms.

A FA-Daa according to the present invention may be represented by the general formula;

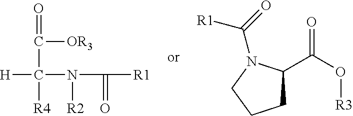

wherein R1 is a hydrocarbon chain comprising 11 to 17 carbons, R2 is either H (i.e. hydrogen) or CH₃ (i.e. methyl group), R3 is either H, or absent, and R4 is an amino acid side chain, wherein the stereo configuration of the chiral carbon atom in the amino acid moiety is in the D-configuration, with the proviso that when R4 is from a non-polar uncharged amino acid, then R1 comprises 11, 13, 15 or 17 carbon atoms and when R4 is from an acidic amino acid, then R1 comprises 15 or 17 carbon atoms.

In one aspect a FA-Daa according to the present invention may be represented by the general formula:

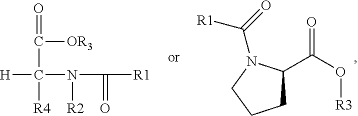

wherein R1 is a hydrocarbon chain comprising 11 to 17 carbon atoms, R2 is either H (i.e. hydrogen) or CH₃ (i.e. methyl group), R3 is either H, or a potassium (K+) or sodium (Na+) salt thereof, and R4 is a amino acid side chain, wherein the stereo configuration of the chiral carbon atom in the amino acid moiety is D, with the proviso that when R4 is from a non-polar uncharged amino acid, then R1 comprises 11, 13, 15 or 17 carbon atoms and when R4 is from an acidic amino acid, then R1 comprises 15 or 17 carbon atoms.

A FA-Daa according to the present invention may be represented by the general formula;

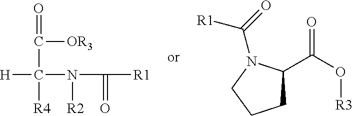

wherein R1 is a hydrocarbon chain comprising 15 to 17 carbons, R2 is either H (i.e. hydrogen) or CH₃ (i.e. methyl group), R3 is either H, or absent, and R4 is an amino acid side chain, wherein the stereo configuration of the chiral carbon atom in the amino acid moiety is in the D-configuration, with the proviso that when R4 is from a non-polar uncharged amino acid, then R1 comprises 15 carbon atoms and when R4 is from an acidic amino acid, then R1 comprises 15 carbon atoms.

In one aspect a FA-Daa according to the present invention may be represented by the general formula:

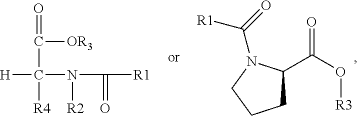

wherein R1 is a hydrocarbon chain comprising 13 to 15 carbon atoms, R2 is either H (i.e. hydrogen) or CH₃ (i.e. methyl group), R3 is either H, or a potassium (K+) or sodium (Na+) salt thereof, and R4 is a amino acid side chain, wherein the stereo configuration of the chiral carbon atom in the amino acid moiety is D, with the proviso that when R4 is from a non-polar uncharged amino acid, then R1 comprises 15 carbon atoms and when R4 is from an acidic amino acid, then R1 comprises 15 carbon atoms.

A FA-Daa according to the present invention may be represented by the general formula;

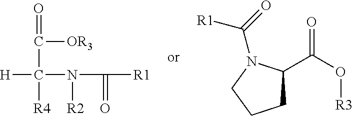

wherein R1 is a hydrocarbon chain comprising 13 to 15 carbons, R2 is either H (i.e. hydrogen) or CH₃ (i.e. methyl group), R3 is either H, or absent, and R4 is an amino acid side chain, wherein the stereo configuration of the chiral carbon atom in the amino acid moiety is in the D-configuration, with the proviso that when R4 is from a non-polar uncharged amino acid, then R1 comprises 13 carbon atoms and when R4 is from an acidic amino acid, then R1 comprises 13 carbon atoms.

In one aspect a FA-Daa according to the present invention may be represented by the general formula:

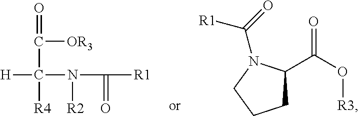

wherein R1 is a hydrocarbon chain comprising 13 to 15 carbon atoms, R2 is either H (i.e. hydrogen) or CH₃ (i.e. methyl group), R3 is either H, or a potassium (K+) or sodium (Na+) salt thereof, and R4 is a amino acid side chain, wherein the stereo configuration of the chiral carbon atom in the amino acid moiety is D, with the proviso that when R4 is from a non-polar uncharged amino acid, then R1 comprises 13 carbon atoms and when R4 is from an acidic amino acid, then R1 comprises 13 carbon atoms.

A FA-Daa according to the present invention may be represented by the general formula;

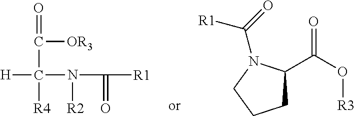

wherein R1 is a hydrocarbon chain comprising 11 to 17 carbons, R2 is either H (i.e. hydrogen) or CH₃ (i.e. methyl group), R3 is either H, or absent, and R4 is an amino acid side chain, wherein the stereo configuration of the chiral carbon atom in the amino acid moiety is in the D-configuration, with the proviso that when R4 is from a non-polar uncharged amino acid, then R1 comprises 11, 13, 15 or 17 carbon atoms and when R4 is from an acidic amino acid, then R1 comprises 15 or 17 carbon atoms.

In one aspect a FA-Daa according to the present invention may be represented by the general formula:

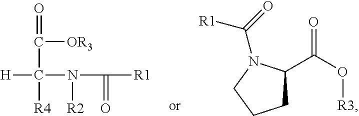

wherein R1 is a hydrocarbon chain comprising 11 to 17 carbon atoms, R2 is either H (i.e. hydrogen) or CH₃ (i.e. methyl group), R3 is either H, or a potassium (K+) or sodium (Na+) salt thereof, and R4 is a amino acid side chain, wherein the stereo configuration of the chiral carbon atom in the amino acid moiety is D, with the proviso that when R4 is from a non-polar uncharged amino acid, then R1 comprises 11, 13, 15 or 17 carbon atoms and when R4 is from an acidic amino acid, then R1 comprises 15 or 17 carbon atoms.

In one aspect FA-Daa according to the present invention may be represented by the general formula;

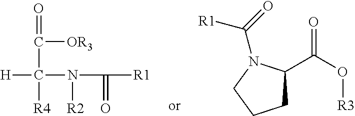

wherein R1 is a hydrocarbon chain comprising 11 to 17 carbons, R2 is either H (i.e. hydrogen) or CH₃ (i.e. methyl group), R3 is either H, or a salt thereof, and R4 is an amino acid side chain of a non-polar uncharged amino acid, wherein the stereo configuration of the chiral carbon atom in the amino acid moiety, is D.

In one aspect a FA-Daa according to the present invention may be represented by the general formula:

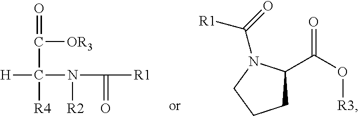

wherein R1 is a hydrocarbon chain comprising 11 to 17 carbon atoms, R2 is either H (i.e. hydrogen) or CH₃ (i.e. methyl group), R3 is either H, or a potassium (K+) o rsodium (Na+) salt thereof, and R4 is a amino acid side chain of a non-polar uncharged amino acid, wherein the stereo configuration of the chiral carbon atom in the amino acid moiety, is D.

A FA-Daa according to the present invention may be represented by the general formula;

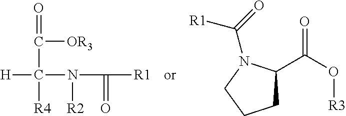

wherein R1 is a hydrocarbon chain comprising 11 to 17 carbons, R2 is either H (i.e. hydrogen) or CH₃ (i.e. methyl group), R3 is either H, or absent, and R4 is an amino acid side chain, wherein the stereo configuration of the chiral carbon atom in the amino acid moiety is in the D-configuration, with the proviso that when R4 is from a non-polar uncharged amino acid selected from the group consisting of: Alanine, Isoleucine, Leucine, Proline and Valine, then R1 comprises 11, 13, 15 or 17 carbon atoms and when R4 is from an acidic amino acid, then R1 comprises 15 or 17 carbon atoms.

In one aspect a FA-Daa according to the present invention may be represented by the general formula:

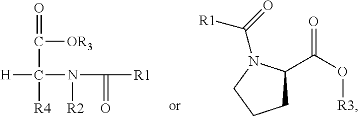

wherein R1 is a hydrocarbon chain comprising 11 to 17 carbon atoms, R2 is either H (i.e. hydrogen) or CH₃ (i.e. methyl group), R3 is either H, or a potassium (K+) or sodium (Na+) salt thereof, and R4 is a amino acid side chain, wherein the stereo configuration of the chiral carbon atom in the amino acid moiety is D, with the proviso that when R4 is from a non-polar uncharged amino acid selected from the group consisting of: Alanine, Isoleucine, Leucine, Proline and Valine, then R1 comprises 11, 13, 15 or 17 carbon atoms and when R4 is from an acidic amino acid, then R1 comprises 15 or 17 carbon atoms.

A FA-Daa according to the present invention may be represented by the general formula;

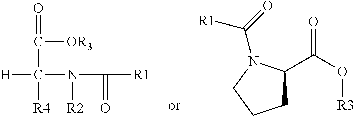

wherein R1 is a hydrocarbon chain comprising 15 to 17 carbons, R2 is either H (i.e. hydrogen) or CH₃ (i.e. methyl group), R3 is either H, or absent, and R4 is an amino acid side chain, wherein the stereo configuration of the chiral carbon atom in the amino acid moiety is in the D-configuration, with the proviso that when R4 is from a non-polar uncharged amino acid selected from the group consisting of: Alanine, Isoleucine, Leucine, Proline and Valine, then R1 comprises 15 carbon atoms and when R4 is from an acidic amino acid, then R1 comprises 15 carbon atoms.

In one aspect a FA-Daa according to the present invention may be represented by the general formula:

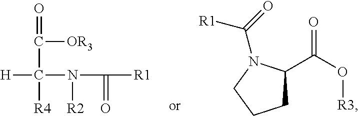

wherein R1 is a hydrocarbon chain comprising 13 to 15 carbon atoms, R2 is either H (i.e. hydrogen) or CH₃ (i.e. methyl group), R3 is either H, or a potassium (K+) or sodium (Na+) salt thereof, and R4 is a amino acid side chain, wherein the stereo configuration of the chiral carbon atom in the amino acid moiety is D, with the proviso that when R4 is from a non-polar uncharged amino acid selected from the group consisting of: Alanine, Isoleucine, Leucine, Proline and Valine, then R1 comprises 15 carbon atoms and when R4 is from an acidic amino acid, then R1 comprises 15 carbon atoms.

A FA-Daa according to the present invention may be represented by the general formula;

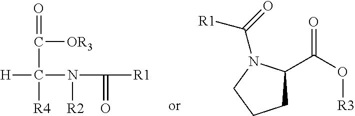

wherein R1 is a hydrocarbon chain comprising 13 to 15 carbons, R2 is either H (i.e. hydrogen) or CH₃ (i.e. methyl group), R3 is either H, or absent, and R4 is an amino acid side chain, wherein the stereo configuration of the chiral carbon atom in the amino acid moiety is in the D-configuration, with the proviso that when R4 is from a non-polar uncharged amino acid selected from the group consisting of: Alanine, Isoleucine, Leucine, Proline and Valine, then R1 comprises 13 carbon atoms and when R4 is from an acidic amino acid, then R1 comprises 13 carbon atoms.

In one aspect a FA-Daa according to the present invention may be represented by the general formula:

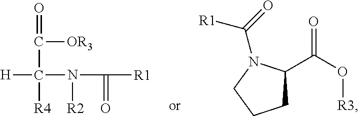

wherein R1 is a hydrocarbon chain comprising 13 to 15 carbon atoms, R2 is either H (i.e. hydrogen) or CH₃ (i.e. methyl group), R3 is either H, or a potassium (K+) or sodium (Na+) salt thereof, and R4 is a amino acid side chain, wherein the stereo configuration of the chiral carbon atom in the amino acid moiety is D, with the proviso that when R4 is from a non-polar uncharged amino acid selected from the group consisting of: Alanine, Isoleucine, Leucine, Proline and Valine, then R1 comprises 13 carbon atoms and when R4 is from an acidic amino acid, then R1 comprises 13 carbon atoms.

A FA-Daa according to the present invention may be represented by the general formula;

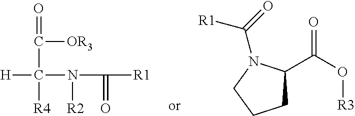

wherein R1 is a hydrocarbon chain comprising 11 to 17 carbons, R2 is either H (i.e. hydrogen) or CH₃ (i.e. methyl group), R3 is either H, or absent, and R4 is an amino acid side chain, wherein the stereo configuration of the chiral carbon atom in the amino acid moiety is in the D-configuration, with the proviso that when R4 is from a non-polar uncharged amino acid selected from the group consisting of: Alanine, Isoleucine, Leucine, Proline and Valine, then R1 comprises 11, 13, 15 or 17 carbon atoms and when R4 is from an acidic amino acid, then R1 comprises 15 or 17 carbon atoms.

In one aspect a FA-Daa according to the present invention may be represented by the general formula:

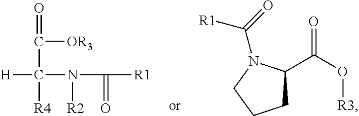

wherein R1 is a hydrocarbon chain comprising 11 to 17 carbon atoms, R2 is either H (i.e. hydrogen) or CH₃ (i.e. methyl group), R3 is either H, or a potassium (K+) or sodium (Na+) salt thereof, and R4 is a amino acid side chain, wherein the stereo configuration of the chiral carbon atom in the amino acid moiety is D, with the proviso that when R4 is from a non-polar uncharged amino acid selected from the group consisting of: Alanine, Isoleucine, Leucine, Proline and Valine, then R1 comprises 11, 13, 15 or 17 carbon atoms and when R4 is from an acidic amino acid, then R1 comprises 15 or 17 carbon atoms.

A FA-Daa according to the present invention may be represented by the general formula;

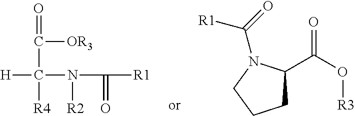

wherein R1 is a hydrocarbon chain comprising 15 to 17 carbons, R2 is either H (i.e. hydrogen) or CH₃ (i.e. methyl group), R3 is either H, or absent, and R4 is an amino acid side chain, wherein the stereo configuration of the chiral carbon atom in the amino acid moiety is in the D-configuration, with the proviso that when R4 is from a non-polar uncharged amino acid selected from the group consisting of: Alanine, Isoleucine, Leucine, Proline and Valine, then R1 comprises 15 carbon atoms and when R4 is from an acidic amino acid, then R1 comprises 15 carbon atoms.

In one aspect a FA-Daa according to the present invention may be represented by the general formula:

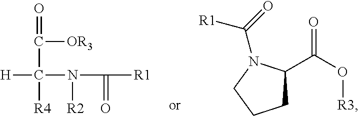

wherein R1 is a hydrocarbon chain comprising 13 to 15 carbon atoms, R2 is either H (i.e. hydrogen) or CH₃ (i.e. methyl group), R3 is either H, or a potassium (K+) or sodium (Na+) salt thereof, and R4 is a amino acid side chain, wherein the stereo configuration of the chiral carbon atom in the amino acid moiety is D, with the proviso that when R4 is from a non-polar uncharged amino acid selected from the group consisting of: Alanine, Isoleucine, Leucine, Proline and Valine, then R1 comprises 15 carbon atoms and when R4 is from an acidic amino acid, then R1 comprises 15 carbon atoms.

A FA-Daa according to the present invention may be represented by the general formula;

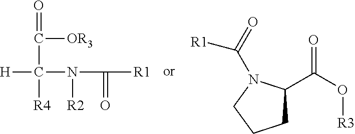

wherein R1 is a hydrocarbon chain comprising 13 to 15 carbons, R2 is either H (i.e. hydrogen) or CH₃ (i.e. methyl group), R3 is either H, or absent, and R4 is an amino acid side chain, wherein the stereo configuration of the chiral carbon atom in the amino acid moiety is in the D-configuration, with the proviso that when R4 is from a non-polar uncharged amino acid selected from the group consisting of: Alanine, Isoleucine, Leucine, Proline and Valine, then R1 comprises 13 carbon atoms and when R4 is from an acidic amino acid, then R1 comprises 13 carbon atoms.

In one aspect a FA-Daa according to the present invention may be represented by the general formula:

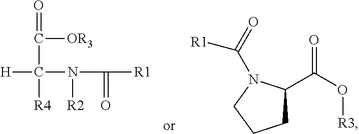

wherein R1 is a hydrocarbon chain comprising 13 to 15 carbon atoms, R2 is either H (i.e. hydrogen) or CH₃ (i.e. methyl group), R3 is either H, or a potassium (K+) or sodium (Na+) salt thereof, and R4 is a amino acid side chain, wherein the stereo configuration of the chiral carbon atom in the amino acid moiety is D, with the proviso that when R4 is from a non-polar uncharged amino acid selected from the group consisting of: Alanine, Isoleucine, Leucine, Proline and Valine, then R1 comprises 13 carbon atoms and when R4 is from an acidic amino acid, then R1 comprises 13 carbon atoms.

A FA-Daa according to the present invention may be represented by the general formula;

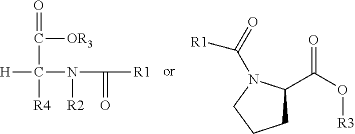

wherein R1 is a hydrocarbon chain comprising 11 to 17 carbons, R2 is either H (i.e. hydrogen) or CH₃ (i.e. methyl group), R3 is either H, or absent, and R4 is an amino acid side chain, wherein the stereo configuration of the chiral carbon atom in the amino acid moiety is in the D-configuration, with the proviso that when R4 is from a non-polar uncharged amino acid selected from the group consisting of: Alanine, Isoleucine, Leucine, Proline and Valine, then R1 comprises 11, 13, 15 or 17 carbon atoms and when R4 is from an acidic amino acid, then R1 comprises 15 or 17 carbon atoms.

In one aspect a FA-Daa according to the present invention may be represented by the general formula:

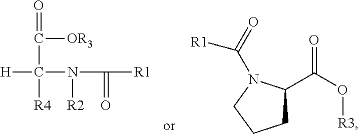

wherein R1 is a hydrocarbon chain comprising 11 to 17 carbon atoms, R2 is either H (i.e. hydrogen) or CH₃ (i.e. methyl group), R3 is either H, or a potassium (K+) or sodium (Na+) salt thereof, and R4 is a amino acid side chain, wherein the stereo configuration of the chiral carbon atom in the amino acid moiety is D, with the proviso that when R4 is from a non-polar uncharged amino acid selected from the group consisting of: Alanine, Isoleucine, Leucine, Proline and Valine, then R1 comprises 11, 13, 15 or 17 carbon atoms and when R4 is from an acidic amino acid, then R1 comprises 15 or 17 carbon atoms.

In one aspect FA-Daa according to the present invention may be represented by the general formula;

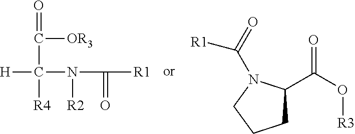

wherein R1 is a hydrocarbon chain comprising 11 to 17 carbons, R2 is either H (i.e. hydrogen) or CH₃ (i.e. methyl group), R3 is either H, or a salt thereof, and R4 is an amino acid side chain of a non-polar uncharged amino acid selected from the group consisting of: Alanine, Isoleucine, Leucine, Proline and Valine, wherein the stereo configuration of the chiral carbon atom in the amino acid moiety, is D.

In one aspect a FA-Daa according to the present invention may be represented by the general formula:

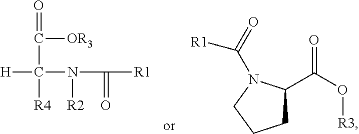

wherein R1 is a hydrocarbon chain comprising 11 to 17 carbon atoms, R2 is either H (i.e. hydrogen) or CH₃ (i.e. methyl group), R3 is either H, or a potassium (K+) o rsodium (Na+) salt thereof, and R4 is a amino acid side chain of a non-polar uncharged amino acid selected from the group consisting of: Alanine, Isoleucine, Leucine, Proline and Valine, wherein the stereo configuration of the chiral carbon atom in the amino acid moiety, is D.

In one aspect FA-Daa according to the present invention may be represented by the general formula;

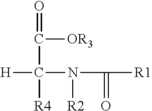

wherein R1 is a hydrocarbon chain comprising 13 to 17 carbons, R2 is either H (i.e. hydrogen) or CH₃ (i.e. methyl group), R3 is either H, or a salt thereof, and R4 is an amino acid side chain of an non-polar uncharged amino acid, wherein the stereo configuration of the chiral carbon atom in the amino acid moiety, is D.

In one aspect a FA-Daa according to the present invention may be represented by the general formula:

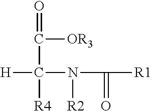

wherein R1 is a hydrocarbon chain comprising 13 to 17 carbon atoms, R2 is either H (i.e. hydrogen) or CH₃ (i.e. methyl group), R3 is either H, or a potassium (K+) or sodium (Na+) salt thereof, and R4 is a amino acid side chain of an non-polar uncharged amino acid, wherein the stereo configuration of the chiral carbon atom in the amino acid moiety, is D.

In one aspect FA-Daa according to the present invention may be represented by the general formula;

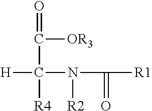

wherein R1 is a hydrocarbon chain comprising 13 to 17 carbons, R2 is either H (i.e. hydrogen) or CH₃ (i.e. methyl group), R3 is either H, or a salt thereof, and R4 is an amino acid side chain of an non-polar uncharged amino acid, wherein the stereo configuration of the chiral carbon atom in the amino acid moiety, is D.

In one aspect a FA-Daa according to the present invention may be represented by the general formula:

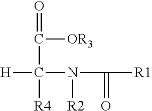

wherein R1 is a hydrocarbon chain comprising 13 to 17 carbon atoms, R2 is either H (i.e. hydrogen) or CH₃ (i.e. methyl group), R3 is either H, or a potassium (K+) or sodium (Na+) salt thereof, and R4 is a amino acid side chain of an non-polar uncharged amino acid, wherein the stereo configuration of the chiral carbon atom in the amino acid moiety, is D.

In one aspect FA-Daa according to the present invention may be represented by the general formula;

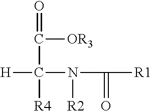

wherein R1 is a hydrocarbon chain comprising 15 to 17 carbons, R2 is either H (i.e. hydrogen) or CH₃ (i.e. methyl group), R3 is either H, or a salt thereof, and R4 is an amino acid side chain of an acidic amino acid, wherein the stereo configuration of the chiral carbon atom in the amino acid moiety, is D.

In one aspect a FA-Daa according to the present invention may be represented by the general formula:

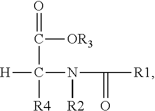

wherein R1 is a hydrocarbon chain comprising 15 to 17 carbon atoms, R2 is either H (i.e. hydrogen) or CH₃ (i.e. methyl group), R3 is either H, or a potassium (K+) or sodium (Na+) salt thereof, and R4 is a amino acid side chain of an acidic amino acid, wherein the stereo configuration of the chiral carbon atom in the amino acid moiety, is D.

In one aspect FA-Daa according to the present invention may be represented by the general formula;

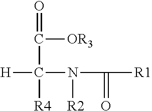

wherein R1 is a hydrocarbon chain comprising 17 carbons, R2 is either H (i.e. hydrogen) or CH₃ (i.e. methyl group), R3 is either H, or a salt thereof, and R4 is an amino acid side chain of an acidic amino acid, wherein the stereo configuration of the chiral carbon atom in the amino acid moiety, is D.

In one aspect a FA-Daa according to the present invention may be represented by the general formula:

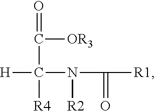

wherein R1 is a hydrocarbon chain comprising 17 carbon atoms, R2 is either H (i.e. hydrogen) or CH₃ (i.e. methyl group), R3 is either H, or a potassium (K+) or sodium (Na+) salt thereof, and R4 is a amino acid side chain of an acidic amino acid, wherein the stereo configuration of the chiral carbon atom in the amino acid moiety, is D.
In one aspect FA-Daa according to the present invention may be represented by the general formula;

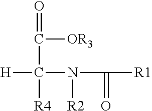

wherein R1 is a hydrocarbon chain comprising 15 carbons, R2 is either H (i.e. hydrogen) or CH₃ (i.e. methyl group), R3 is either H, or a salt thereof, and R4 is an amino acid side chain of an acidic amino acid, wherein the stereo configuration of the chiral carbon atom in the amino acid moiety, is D.

In one aspect a FA-Daa according to the present invention may be represented by the general formula:

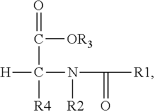

wherein R1 is a hydrocarbon chain comprising 15 carbon atoms, R2 is either H (i.e. hydrogen) or CH₃ (i.e. methyl group), R3 is either H, or a potassium (K+) or sodium (Na+) salt thereof, and R4 is a amino acid side chain of an acidic amino acid, wherein the stereo configuration of the chiral carbon atom in the amino acid moiety, is D.

In one aspect FA-Daa according to the present invention may be represented by the general formula;

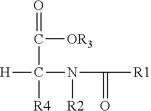

wherein R1 is a hydrocarbon chain comprising 15 to 17 carbons, R2 is either H (i.e. hydrogen) or CH₃ (i.e. methyl group), R3 is either H, or a salt thereof, and R4 is an amino acid side chain of an acidic amino acid selected from the group consisting of: Aspartic acid and Glutamic acid, wherein the stereo configuration of the chiral carbon atom in the amino acid moiety, is D.

In one aspect a FA-Daa according to the present invention may be represented by the general formula:

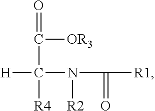

wherein R1 is a hydrocarbon chain comprising 15 to 17 carbon atoms, R2 is either H (i.e. hydrogen) or CH₃ (i.e. methyl group), R3 is either H, or a potassium (K+) or sodium (Na+) salt thereof, and R4 is a amino acid side chain of an acidic amino acid selected from the group consisting of: Aspartic acid and Glutamic acid, wherein the stereo configuration of the chiral carbon atom in the amino acid moiety, is D.

In one aspect FA-Daa according to the present invention may be represented by the general formula;

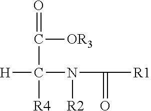

wherein R1 is a hydrocarbon chain comprising 17 carbons, R2 is either H (i.e. hydrogen) or CH₃ (i.e. methyl group), R3 is either H, or a salt thereof, and R4 is an amino acid side chain of an acidic amino acid selected from the group consisting of: Aspartic acid and Glutamic acid, wherein the stereo configuration of the chiral carbon atom in the amino acid moiety, is D.

In one aspect a FA-Daa according to the present invention may be represented by the general formula:

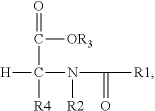

wherein R1 is a hydrocarbon chain comprising 17 carbon atoms, R2 is either H (i.e. hydrogen) or CH₃ (i.e. methyl group), R3 is either H, or a potassium (K+) or sodium (Na+) salt thereof, and R4 is a amino acid side chain of an acidic amino acid selected from the group consisting of: Aspartic acid and Glutamic acid, wherein the stereo configuration of the chiral carbon atom in the amino acid moiety, is D.

In one aspect FA-Daa according to the present invention may be represented by the general formula;

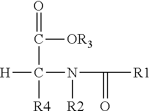

wherein R1 is a hydrocarbon chain comprising 15 carbons, R2 is either H (i.e. hydrogen) or CH₃ (i.e. methyl group), R3 is either H, or a salt thereof, and R4 is an amino acid side chain of an acidic amino acid selected from the group consisting of: Aspartic acid and Glutamic acid, wherein the stereo configuration of the chiral carbon atom in the amino acid moiety, is D.

In one aspect a FA-Daa according to the present invention may be represented by the general formula:

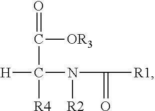

wherein R1 is a hydrocarbon chain comprising 15 carbon atoms, R2 is either H (i.e. hydrogen) or CH₃ (i.e. methyl group), R3 is either H, or a potassium (K+) or sodium (Na+) salt thereof, and R4 is a amino acid side chain of an acidic amino acid selected from the group consisting of: Aspartic acid and Glutamic acid, wherein the stereo configuration of the chiral carbon atom in the amino acid moiety, is D.

In one aspect a FA-Daa according to this invention may be chosen from the group consisting of formula (m) and (n) wherein R1 is a hydrocarbon chain comprising 15 to 17 carbons, R2 is either H (i.e. hydrogen) or CH₃ (i.e. methyl group), and R3 is either H, or a salt thereof.

In one aspect a FA-Daa according to this invention may be chosen from the group consisting of formula (m) and (n) wherein R1 is a hydrocarbon chain comprising 15 to 17 carbon atoms, R2 is either H (i.e. hydrogen) or CH₃ (i.e. methyl group), and R3 is either H, or a sodium (Na+) or potassium (K+) salt thereof.

In one aspect a FA-Daa according to this invention may be chosen from the group consisting of formula (m) and (n) wherein R1 is a hydrocarbon chain comprising 15 carbons, R2 is either H (i.e. hydrogen) or CH₃ (i.e. methyl group), and R3 is either H, or a salt thereof. In one aspect a FA-Daa according to this invention may be chosen from the group consisting of formula (m) and (n) wherein R1 is a hydrocarbon chain comprising 17 carbons, R2 is either H (i.e. hydrogen) or CH₃ (i.e. methyl group), and R3 is either H, or a salt thereof.

In one aspect a FA-Daa according to this invention may be chosen from the group consisting of formula (m) and (n) wherein R1 is a hydrocarbon chain comprising 15 to 17 carbon atoms, R2 is either H (i.e. hydrogen) or CH₃ (i.e. methyl group), and R3 is either H, or a sodium (Na+) or potassium (K+) salt thereof. In one aspect a FA-Daa according to this invention may be chosen from the group consisting of formula (m) and (n) wherein R1 is a hydrocarbon chain comprising 15 carbon atoms, R2 is either H (i.e. hydrogen) or CH₃ (i.e. methyl group), and R3 is either H, or a sodium (Na+) or potassium (K+) salt thereof. In one aspect a FA-Daa according to this invention may be chosen from the group consisting of formula (m) and (n) wherein R1 is a hydrocarbon chain comprising 17 carbon atoms, R2 is either H (i.e. hydrogen) or CH₃ (i.e. methyl group), and R3 is either H, or a sodium (Na+) or potassium (K+) salt thereof.

In one aspect a FA-Daa according to this invention may be chosen from the group consisting of (m) and (n) wherein R1 is a hydrocarbon chain comprising 15 to 17 carbons, R2 is either H (i.e. hydrogen) or CH₃ (i.e. methyl group), and R3 is either H, or a salt thereof. In one aspect a FA-Daa according to this invention may be chosen from the group consisting of (m) and (n) wherein R1 is a hydrocarbon chain comprising 15 to 17 carbon atoms, R2 is either H (i.e. hydrogen) or CH₃ (i.e. methyl group), and R3 is either H, or a sodium (Na+) or potassium (K+) salt thereof.

In one aspect a FA-Daa according to this invention may be chosen from the group consisting of formula (h), (i), (j), (k) and (l) wherein R1 is a hydrocarbon chain comprising 11 to 17 carbons, R2 is either H (i.e. hydrogen) or CH₃ (i.e. methyl group), and R3 is either H, or a salt thereof. In one aspect a FA-Daa according to this invention may be chosen from the group consisting of formula (h), (i), (j), (k) and (l) wherein R1 is a hydrocarbon chain comprising 11 carbons, R2 is either H (i.e. hydrogen) or CH₃ (i.e. methyl group), and R3 is either H, or a salt thereof. In one aspect a FA-Daa according to this invention may be chosen from the group consisting of formula (h), (i), (j), (k) and (l) wherein R1 is a hydrocarbon chain comprising 13 carbons, R2 is either H (i.e. hydrogen) or CH₃ (i.e. methyl group), and R3 is either H, or a salt thereof. In one aspect a FA-Daa according to this invention may be chosen from the group consisting of formula (h), (i), (j), (k) and (l) wherein R1 is a hydrocarbon chain comprising 13 carbons, R2 is either H (i.e. hydrogen) or CH₃ (i.e. methyl group), and R3 is either H, or a salt thereof. In one aspect a FA-Daa according to this invention may be chosen from the group consisting of formula (h), (i), (j), (k) and (l) wherein R1 is a hydrocarbon chain comprising 15 carbons, R2 is either H (i.e. hydrogen) or CH₃ (i.e. methyl group), and R3 is either H, or a salt thereof. In one aspect a FA-Daa according to this invention may be chosen from the group consisting of formula (h), (i), (j), (k) and (l) wherein R1 is a hydrocarbon chain comprising 17 carbons, R2 is either H (i.e. hydrogen) or CH₃ (i.e. methyl group), and R3 is either H, or a salt thereof.

In one aspect a FA-Daa according to this invention may be chosen from the group consisting of formula (h), (i), (j), (k) and (l) wherein R1 is a hydrocarbon chain comprising 11 to 17 carbon atoms, R2 is either H (i.e. hydrogen) or CH₃ (i.e. methyl group), and R3 is either H, or a sodium (Na+) or potassium (K+) salt thereof. In one aspect a FA-Daa according to this invention may be chosen from the group consisting of formula (h), (i), (j), (k) and (l) wherein R1 is a hydrocarbon chain comprising 11 carbon atoms, R2 is either H (i.e. hydrogen) or CH₃ (i.e. methyl group), and R3 is either H, or a sodium (Na+) or potassium (K+) salt thereof. In one aspect a FA-Daa according to this invention may be chosen from the group consisting of formula (h), (i), (j), (k) and (l) wherein R1 is a hydrocarbon chain comprising 13 carbon atoms, R2 is either H (i.e. hydrogen) or CH₃ (i.e. methyl group), and R3 is either H, or a sodium (Na+) or potassium (K+) salt thereof. In one aspect a FA-Daa according to this invention may be chosen from the group consisting of formula (h), (i), (j), (k) and (l) wherein R1 is a hydrocarbon chain comprising 15 carbon atoms, R2 is either H (i.e. hydrogen) or CH₃ (i.e. methyl group), and R3 is either H, or a sodium (Na+) or potassium (K+) salt thereof. In one aspect a FA-Daa according to this invention may be chosen from the group consisting of formula (h), (i), (j), (k) and (l) wherein R1 is a hydrocarbon chain comprising 17 carbon atoms, R2 is either H (i.e. hydrogen) or CH₃ (i.e. methyl group), and R3 is either H, or a sodium (Na+) or potassium (K+) salt thereof.

In one aspect a FA-Daa according to this invention may be chosen from the group consisting of formula (h), (i), (j), (k) and (l) wherein R1 is a hydrocarbon chain comprising 13 to 17 carbons, R2 is either H (i.e. hydrogen) or CH₃ (i.e. methyl group), and R3 is either H, or a salt thereof.

In one aspect a FA-Daa according to this invention may be chosen from the group consisting of formula (h), (i), (j), (k) and (l) wherein R1 is a hydrocarbon chain comprising 13 to 17 carbon atoms, R2 is either H (i.e. hydrogen) or CH₃ (i.e. methyl group), and R3 is either H, or a sodium (Na+) or potassium (K+) salt thereof.

In one aspect a FA-Daa according to this invention may be chosen from the group consisting of (h), (i), (j), (k) and (l) wherein R1 is a hydrocarbon chain comprising 15 to 17 carbons, R2 is either H (i.e. hydrogen) or CH₃ (i.e. methyl group), and R3 is either H, or a salt thereof.

In one aspect a FA-Daa according to this invention may be chosen from the group consisting of (h), (i), (j), (k) and (l) wherein R1 is a hydrocarbon chain comprising 15 to 17 carbon atoms, R2 is either H (i.e. hydrogen) or CH₃ (i.e. methyl group), and R3 is either H, or a sodium (Na+) or potassium (K+) salt thereof.

In one aspect the formulas (h), (i), (j), (k) and (l) are present as follows, wherein R1 is a hydrocarbon chain comprising 11 to 17 carbon atoms, R2 is either H (i.e. hydrogen) or CH₃ (i.e. methyl group), and R3 is either H, or a sodium (Na+) or potassium (K+) salt thereof:

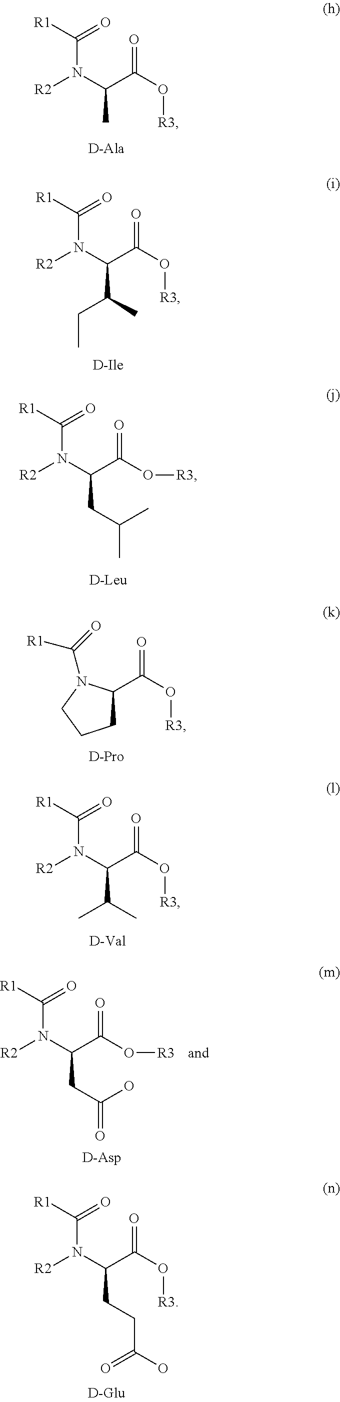

For illustration purposes, the L-Proline FA-Laa structure has been shown, wherein R1 is a hydrocarbon chain comprising 11 to 17 carbon atoms, R2 is either H (i.e. hydrogen) or CH₃ (i.e. methyl group), and R3 is either H, or a sodium (Na+) or potassium (K+) salt thereof:

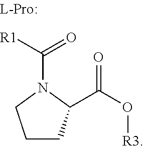

In one aspect a FA-Daa can be selected from the group consisting of: Sodium or potassium lauroyl D-alaninate, N-dodecanoyl-D-alanine, Sodium or potassium myristoyl D-Alaninate, N-tetradecanoyl D-Alanine, Sodium or potassium palmitoyl D-Alaninate, N-hexadecanoyl D-Alanine, Sodium or potassium stearoyl D-Alaninate and N-octadecanoyl D-Alanine.

In one aspect a FA-Daa can be selected from the group consisting of: Sodium or potassium lauroyl D-Isoleucinate, N-dodecanoyl-D-Isoleucine, Sodium or potassium myristoyl D-Isoleucinate, N-tetradecanoyl D-Isoleucine, Sodium or potassium palmitoyl D-Isoleucinate, N-hexadecanoyl D-Isoleucine, Sodium or potassium stearoyl D-Isoleucinate and N-octadecanoyl D-Isoleucine.

In one aspect a FA-Daa can be selected from the group consisting of: Sodium or potassium lauroyl D-Leucinate, N-dodecanoyl-D-Leucine, Sodium or potassium myristoyl D-Leucinate, N-tetradecanoyl D-Leucine, Sodium or potassium palmitoyl D-Leucinate, N-hexadecanoyl D-Leucine, Sodium or potassium stearoyl D-Leucinate and N-octadecanoyl D-Leucine.

In one aspect a FA-Daa can be selected from the group consisting of: Sodium or potassium lauroyl D-Prolinate, N-dodecanoyl-D-Proline, Sodium or potassium myristoyl D-Prolinate, N-tetradecanoyl D-Proline, Sodium or potassium palmitoyl D-Prolinate, N-hexadecanoyl D-Proline, Sodium or potassium stearoyl D-Prolinate and N-octadecanoyl D-Proline.

In one aspect a FA-Daa can be selected from the group consisting of: Sodium or potassium lauroyl D-Valinate, N-dodecanoyl-D-Valine, Sodium or potassium myristoyl D-Valinate, N-tetradecanoyl D-Valine, Sodium or potassium palmitoyl D-Valinate, N-hexadecanoyl D-Valine, Sodium or potassium stearoyl D-Valinate and N-octadecanoyl D-Valine.

In one aspect a FA-Daa can be selected from the group consisting of: Sodium or potassium lauroyl D-alaninate, N-dodecanoyl-D-alanine, Sodium or potassium myristoyl D-Alaninate, N-tetradecanoyl D-Alanine, Sodium or potassium palmitoyl D-Alaninate, N-hexadecanoyl D-Alanine, Sodium or potassium stearoyl D-Alaninate, N-octadecanoyl D-Alanine, Sodium or potassium lauroyl D-Isoleucinate, N-dodecanoyl-D-Isoleucine, Sodium or potassium myristoyl D-Isoleucinate, N-tetradecanoyl D-Isoleucine, Sodium or potassium palmitoyl D-Isoleucinate, N-hexadecanoyl D-Isoleucine, Sodium or potassium stearoyl D-Isoleucinate, N-octadecanoyl D-Isoleucine, Sodium or potassium lauroyl D-Leucinate, N-dodecanoyl-D-Leucine, Sodium or potassium myristoyl D-Leucinate, N-tetradecanoyl D-Leucine, Sodium or potassium palmitoyl D-Leucinate, N-hexadecanoyl D-Leucine, Sodium or potassium stearoyl D-Leucinate, N-octadecanoyl D-Leucine, Sodium or potassium lauroyl D-Prolinate, N-dodecanoyl-D-Proline, Sodium or potassium myristoyl D-Prolinate, N-tetradecanoyl D-Proline, Sodium or potassium palmitoyl D-Prolinate, N-hexadecanoyl D-Proline, Sodium or potassium stearoyl D-Prolinate, N-octadecanoyl D-Proline, Sodium or potassium lauroyl D-Valinate, N-dodecanoyl-D-Valine, Sodium or potassium myristoyl D-Valinate, N-tetradecanoyl D-Valine, Sodium or potassium palmitoyl D-Valinate, N-hexadecanoyl D-Valine, Sodium or potassium stearoyl D-Valinate and N-octadecanoyl D-Valine.

In one aspect a FA-Daa can be selected from the group consisting of: Sodium or potassium lauroyl D-alaninate, N-dodecanoyl-D-alanine, Sodium or potassium lauroyl D-Isoleucinate, N-dodecanoyl D-Isoleucine, Sodium or potassium lauroyl D-Leucinate, N-dodecanoyl-D-Leucine, Sodium or potassium lauroyl D-Prolinate, N-dodecanoyl-D-Proline, Sodium or potassium lauroyl D-Valinate and N-dodecanoyl-D-Valine.

In one aspect a FA-Daa can be selected from the group consisting of: Sodium or potassium lauroyl D-alaninate, N-dodecanoyl-D-alanine, Sodium or potassium lauroyl D-Isoleucinate, N-dodecanoyl-D-Isoleucine, Sodium or potassium lauroyl D-Leucinate, N-dodecanoyl-D-Leucine, Sodium or potassium lauroyl D-Prolinate, N-dodecanoyl-D-Proline, Sodium or potassium lauroyl D-Valinate and N-dodecanoyl-D-Valine.

In one aspect a FA-Daa can be selected from the group consisting of: Sodium or potassium myristoyl D-Alaninate, N-tetradecanoyl D-Alanine, Sodium or potassium myristoyl D-Isoleucinate, N-tetradecanoyl D-Isoleucine, Sodium or potassium myristoyl D-Leucinate, N-tetradecanoyl D-Leucine, Sodium or potassium myristoyl D-Prolinate, N-tetradecanoyl D-Proline, Sodium or potassium myristoyl D-Valinate and N-tetradecanoyl D-Valine.

In one aspect a FA-Daa can be selected from the group consisting of: Sodium or potassium palmitoyl D-Alaninate, N-hexadecanoyl D-Alanine, Sodium or potassium palmitoyl D-Isoleucinate, N-hexadecanoyl D-Isoleucine, Sodium or potassium palmitoyl D-Leucinate, N-hexadecanoyl D-Leucine, Sodium or potassium palmitoyl D-Prolinate, N-hexadecanoyl D-Proline, Sodium or potassium palmitoyl D-Valinate and N-hexadecanoyl D-Valine.

In one aspect a FA-Daa can be selected from the group consisting of: Sodium or potassium stearoyl D-Alaninate, N-octadecanoyl D-Alanine, Sodium or potassium stearoyl D-Isoleucinate, N-octadecanoyl D-Isoleucine, Sodium or potassium stearoyl D-Leucinate, N-octadecanoyl D-Leucine, Sodium or potassium stearoyl D-Prolinate, N-octadecanoyl D-Proline, Sodium or potassium stearoyl D-Valinate and N-octadecanoyl D-Valine.

In one aspect a FA-Daa can be selected from the group consisting of: Sodium or potassium lauroyl D-alaninate, N-dodecanoyl-D-alanine, Sodium or potassium lauroyl D-Isoleucinate, N-dodecanoyl D-Isoleucine, Sodium or potassium lauroyl D-Leucinate, N-dodecanoyl-D-Leucine, Sodium or potassium lauroyl D-Prolinate, N-dodecanoyl-D-Proline, Sodium or potassium lauroyl D-Valinate, N-dodecanoyl-D-Valine, Sodium or potassium lauroyl D-alaninate, N-dodecanoyl-D-alanine, Sodium or potassium lauroyl D-Isoleucinate, N-dodecanoyl-D-Isoleucine, Sodium or potassium lauroyl D-Leucinate, N-dodecanoyl-D-Leucine, Sodium or potassium lauroyl D-Prolinate, N-dodecanoyl-D-Proline, Sodium or potassium lauroyl D-Valinate and N-dodecanoyl-D-Valine.

In one aspect a FA-Daa can be selected from the group consisting of: Sodium or potassium palmitoyl D-Aspartate, N-hexadecanoyl D-Aspartic acid, Sodium or potassium palmitoyl D-Glutamate, N-hexadecanoyl D-Glutamic acid, Sodium or potassium stearoyl D-Aspartate, N-octadecanoyl D-Aspartic acid, Sodium or potassium stearoyl D-Glutamate and N-octadecanoyl D-Glutamic acid.

In one aspect a FA-Daa can be selected from the group consisting of: Sodium or potassium palmitoyl D-Aspartate, N-hexadecanoyl D-Aspartic acid, Sodium or potassium palmitoyl D-Glutamate and N-hexadecanoyl D-Glutamic acid.

In one aspect a FA-Daa can be selected from the group consisting of: Sodium or potassium stearoyl D-Aspartate, N-octadecanoyl D-Aspartic acid, Sodium or potassium stearoyl D-Glutamate and N-octadecanoyl D-Glutamic acid.

Modifications of amino acids by acylation are readily performed using acylation agents known in the art that react with the free alpha-amino group of the amino acid.

According to the present invention, the FA-Daa may be part of an oral pharmaceutical composition.

In one aspect of the invention the pharmaceutical composition comprises of at least one therapeutic macromolecule, such as a hydrophilic peptide or protein and at least on FA-Daa and propylene glycol.

In one aspect the amino acid FA-Daa may be used in a liquid or semisolid liquid and surfactant based delivery system. In one aspect the amino acid FA-Daa may be used in a liquid or semisolid liquid and surfactant based delivery system, such as SEDDS, SMEDDS or SNEDDS. In one aspect the amino acid FA-Daa may be used in a solid surfactant based delivery system. In one aspect the amino acid FA-Daa may be used in a solid surfactant based delivery system, such as SEDDS, SMEDDS or SNEDDS. Liquid or semisolid SEDDS, SMEDDS or SNEDDS comprising FA-Daa's according to the invention may be encapsulated with any available soft- or hard capsule technology to result in a solid oral pharmaceutical dosage form. Thus the term “solid” as used herein refers to liquid compositions encapsulated in a soft or hard capsule technology, but also to tablets and multiparticulates.

Liquid or semisolid SEDDS, SMEDDS or SNEDDS according to the invention may be encapsulated with any available soft- or hard capsule technology to result in a solid oral pharmaceutical dosage form which may further comprise enteric or delayed release coatings.

Liquid or semisolid SEDDS, SMEDDS or SNEDDS comprising FA-Daa's according to the invention may be encapsulated with any available soft- or hard capsule technology to result in a solid oral pharmaceutical dosage form which may further comprise enteric or delayed release coatings, such as poly(meth)acrylates, commercially known as Eudragit®.

In one aspect of the invention the pharmaceutical composition is a SEDDS, SMEDDS or SNEDDS, comprising at least one therapeutic macromolecule, such as a hydrophilic peptide or protein and at least one FA-Daa, propylene glycol.

In one aspect the pharmaceutical composition according to the present comprises less than 10% (w/w) water. In one aspect the pharmaceutical composition according to the present comprises less than 9% (w/w) water. In one aspect the pharmaceutical composition according to the present comprises less than 8% (w/w) water. In one aspect the pharmaceutical composition according to the present comprises less than 7% (w/w) water. In one aspect the pharmaceutical composition according to the present comprises less than 6% (w/w) water. In one aspect the pharmaceutical composition according to the present comprises less than 5% (w/w) water. In one aspect the pharmaceutical composition according to the present comprises less than 4% (w/w) water. In one aspect the pharmaceutical composition according to the present comprises less than 3% (w/w) water. In one aspect the pharmaceutical composition according to the present comprises less than 2% (w/w) water. In one aspect the pharmaceutical composition according to the present comprises less than 1% (w/w) water. In one aspect the pharmaceutical composition according to the present comprises less than 0% (w/w) water.

In one aspect the pharmaceutical composition according to the present invention is is a liquid. In one aspect the pharmaceutical composition according to the present invention is is a liquid and comprises less than 10% (w/w) water. In one aspect the pharmaceutical composition according to the present invention is a liquid and comprises less than 9% (w/w) water. In one aspect the pharmaceutical composition according to the present invention is a liquid and comprises less than 8% (w/w) water. In one aspect the pharmaceutical composition according to the present invention is a liquid and comprises less than 7% (w/w) water. In one aspect the pharmaceutical composition according to the present invention is a liquid and comprises less than 6% (w/w) water. In one aspect the pharmaceutical composition according to the present invention is a liquid and comprises less than 5% (w/w) water. In one aspect the pharmaceutical composition according to the present invention is a liquid and comprises less than 4% (w/w) water. In one aspect the pharmaceutical composition according to the present invention is a liquid and comprises less than 3% (w/w) water. In one aspect the pharmaceutical composition according to the present invention is a liquid and comprises less than 2% (w/w) water. In one aspect the pharmaceutical composition according to the present invention is a liquid and comprises less than 1% (w/w) water. In one aspect the pharmaceutical composition according to the present invention is a liquid and comprises less than 0% (w/w) water.

In one aspect of the invention the pharmaceutical composition comprises at least one therapeutic macromoecule. In one aspect a therapeutic macromolecule, such as a hydrophilic peptide or protein according to this invention is a therapeutic active peptide or protein. In one aspect a therapeutic peptide or protein according to this invention is a hydrophilic peptide or protein.

In one aspect a hydrophilic peptide or protein of this invention is a peptide or protein having a solubility of at least 50 mg/mL in water. In one aspect a hydrophilic peptide or protein of this invention is a peptide or protein having a solubility of at least 60 mg/mL in water In one aspect a hydrophilic peptide or protein of this invention is a peptide or protein having a solubility of at least 70 mg/mL in water In one aspect a hydrophilic peptide or protein of this invention is a peptide or protein having a solubility of at least 80 mg/mL in water. In one aspect a hydrophilic peptide or protein of this invention is a peptide or protein having a solubility of at least 90 mg/mL in water. In one aspect a hydrophilic peptide or protein of this invention is a peptide or protein having a solubility of at least 100 mg/mL in water. In one aspect a hydrophilic peptide or protein of this invention is a peptide or protein having a solubility of at least 110 mg/mL in water. In one aspect a hydrophilic peptide or protein of this invention is a peptide or protein having a solubility of at least 120 mg/mL in water. In one aspect a hydrophilic peptide or protein of this invention is a peptide or protein having a solubility of at least 130 mg/mL in water. In one aspect a hydrophilic peptide or protein of this invention is a peptide or protein having a solubility of at least 140 mg/mL in water. In one aspect a hydrophilic peptide or protein of this invention is a peptide or protein having a solubility of at least 150/mL in water. In one aspect a hydrophilic peptide or protein of this invention is a peptide or protein having a solubility of at least 160 mg/mL in water. In one aspect a hydrophilic peptide or protein of this invention is a peptide or protein having a solubility of at least 170 mg/mL in water. In one aspect a hydrophilic peptide or protein of this invention is a peptide or protein having a solubility of at least 180 mg/mL in water. In one aspect a hydrophilic peptide or protein of this invention is a peptide or protein having a solubility of at least 190 mg/mL in water. In one aspect a hydrophilic peptide or protein of this invention is a peptide or protein having a solubility of at least 200 mg/mL in water. In one aspect a hydrophilic peptide or protein of this invention is a peptide or protein having a solubility of at least 210 mg/mL in water. In one aspect a hydrophilic peptide or protein of this invention is a peptide or protein having a solubility of at least 220 mg/mL in water. In one aspect a hydrophilic peptide or protein of this invention is a peptide or protein having a solubility of at least 230 mg/mL in water. In one aspect a hydrophilic peptide or protein of this invention is a peptide or protein having a solubility of at least 240 mg/mL in water.

In one aspect a therapeutic active peptide or protein according to this invention is a peptide or protein of more than 1500 Da. In one aspect a therapeutic active peptide or protein according to this invention is a peptide or protein of more than 1750 Da. In one aspect a therapeutic active peptide or protein according to this invention is a peptide or protein of more than 2000 Da. In one aspect a therapeutic active peptide or protein according to this invention is a peptide or protein of more than 2250 Da. In one aspect a therapeutic active peptide or protein according to this invention is a peptide or protein of more than 2500 Da. In one aspect a therapeutic active peptide or protein according to this invention is a peptide or protein of more than 2750 Da. In one aspect a therapeutic active peptide or protein according to this invention is a peptide or protein of more than 3000 Da. In one aspect a therapeutic active peptide or protein according to this invention is a peptide or protein of more than 3250 Da. In one aspect a therapeutic active peptide or protein according to this invention is a peptide or protein of more than 3500 Da. In one aspect a therapeutic active peptide or protein according to this invention is a peptide or protein of more than 3750 Da. In one aspect a therapeutic active peptide or protein according to this invention is a peptide or protein of more than 4000 Da. In one aspect a therapeutic active peptide or protein according to this invention is a peptide or protein of more than 4250 Da. In one aspect a therapeutic active peptide or protein according to this invention is a peptide or protein of more than 4500 Da. In one aspect a therapeutic active peptide or protein according to this invention is a peptide or protein of more than 4750 Da. In one aspect a therapeutic active peptide or protein according to this invention is a peptide or protein of more than 5000 Da. In one aspect a therapeutic active peptide or protein according to this invention is a peptide or protein of more than 1500 Da. In one aspect a therapeutic active peptide or protein according to this invention is a peptide or protein of between 1500 Da and 5000 Da.

In one aspect a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester, and a polar or semipolar solvent.

In one aspect a pharmaceutical composition according to the present invention is a liquid and comprises a therapeutic hydrophilic protein or polypeptide, at least one fatty acid acylated amino acid, at least one polyglycerol fatty acid ester, further comprising Polyethylene glycol sorbitan fatty acid ester, and a polar or semipolar solvent, wherein the solvent is selected from the group consisting of water and propylene glycol.

In one aspect a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester, wherein said Polyethylene glycol sorbitan fatty acid ester is selected from the group consisting of Tween 20, Tween 40, Tween 60 and Tween 80. In one aspect a pharmaceutical composition according to the present invention is a liquid and comprises a therapeutic hydrophilic protein or polypeptide, at least one fatty acid acylated amino acid, at least one polyglycerol fatty acid ester, further comprising Polyethylene glycol sorbitan fatty acid ester, wherein said Polyethylene glycol sorbitan fatty acid ester is selected from the group consisting of Tween 20, Tween 40, Tween 60 and Tween 80.

In one aspect a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester, and a polar or semipolar solvent, wherein the solvent is selected from the group consisting of water and propylene glycol.

In one aspect a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester, and a polar or semipolar solvent, wherein the Polyethylene glycol sorbitan fatty acid ester is a Polyethylene glycol sorbitan trioleate, commercially known as Tween 85.

In one aspect a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester, and a polar or semipolar solvent, wherein the Polyethylene glycol sorbitan fatty acid ester is a Polyethylene glycol sorbitan trioleate, commercially known as Tween 85 and the solvent is selected form the group consisting of water and propylene glycol.

In one aspect a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan trioleate commercially known as Tween 85 and a polar or semipolar solvent selected from the group consistin of water and propylene glycol, wherein the composition forms a microemulsion after dilution in an aqeous medium.

In one aspect a pharmaceutical composition according to the present invention is a liquid and comprises a therapeutic hydrophilic protein or polypeptide, at least one fatty acid acylated amino acid, at least one polyglycerol fatty acid ester, further comprising Polyethylene glycol sorbitan trioleate commercially known as Tween 85 and a polar or semipolar solvent selected from the group consistin of water and propylene glycol, wherein the composition forms a microemulsion after dilution in an aqeous medium.

In one aspect a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester, and a polar or semipolar solvent, wherein the Polyethylene glycol sorbitan fatty acid ester is a Polyethylene glycol sorbitan trioleate, commercially known as Tween 20.

In one aspect a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester, and a polar or semipolar solvent, wherein the Polyethylene glycol sorbitan fatty acid ester is a Polyethylene glycol sorbitan monolaurate, commercially known as Tween 20 and the solvent is selected form the group consisting of water and propylene glycol.

In one aspect a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan monolaurate commercially known as Tween 20 and a polar or semipolar solvent selected from the group consistin of water and propylene glycol, wherein the composition forms a microemulsion after dilution in an aqeous medium.

In one aspect a pharmaceutical composition according to the present invention is a liquid and comprises a therapeutic hydrophilic protein or polypeptide, at least one fatty acid acylated amino acid, at least one polyglycerol fatty acid ester, further comprising Polyethylene glycol sorbitan monolaurate commercially known as Tween 20 and a polar or semipolar solvent selected from the group consistin of water and propylene glycol, wherein the composition forms a microemulsion after dilution in an aqeous medium.

In one aspect a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester and a polar or semipolar solvent. In one aspect a pharmaceutical composition according to the present invention is a liquid and comprises a therapeutic hydrophilic protein or polypeptide, at least one fatty acid acylated amino acid, at least one polyglycerol fatty acid ester, further comprising Polyethylene glycol sorbitan fatty acid ester and a polar or semipolar solvent.

In one aspect a pharmaceutical composition according to the present invention is a liquid and comprises at least one therapeutic hydrophilic protein or polypeptide, at least one fatty acid acylated amino acid, at least one polyglycerol fatty acid ester, further comprising Polyethylene glycol sorbitan fatty acid ester and a polar or semipolar solvent, wherein said polar or semipolar solvent is selected from the group consisting of water and propylene glycol. In one aspect a pharmaceutical composition according to the present invention is a liquid and comprises a therapeutic hydrophilic protein or polypeptide, at least one fatty acid acylated amino acid, at least one polyglycerol fatty acid ester, further comprising Polyethylene glycol sorbitan fatty acid ester and a polar or semipolar solvent, wherein said polar or semipolar solvent is selected from the group consisting of water and propylene glycol.

In one aspect a pharmaceutical composition according to the present invention is a liquid and comprises at least one therapeutic hydrophilic protein or polypeptide, at least one fatty acid acylated amino acid, at least one polyglycerol fatty acid ester, further comprising sorbitan fatty acid ester and a polar or semipolar solvent (such as water or propylene glycol). In one aspect a pharmaceutical composition according to the present invention is a liquid and comprises a therapeutic hydrophilic protein or polypeptide, at least one fatty acid acylated amino acid, at least one polyglycerol fatty acid ester, further comprising sorbitan fatty acid ester (Span 40), and a polar or semipolar solvent (such as water or propylene glycol).

In one aspect a pharmaceutical composition according to the present invention is a liquid and comprises at least one therapeutic hydrophilic protein or polypeptide, at least one fatty acid acylated amino acid, at least one polyglycerol fatty acid ester, further comprising sorbitan fatty acid ester, wherein said sorbitan fatty acid ester is Span 40. In one aspect a pharmaceutical composition according to the present invention is a liquid and comprises a therapeutic hydrophilic protein or polypeptide, at least one fatty acid acylated amino acid, at least one polyglycerol fatty acid ester, further comprising sorbitan fatty acid ester, wherein said sorbitan fatty acid ester is sorbitan mono palmitate commercially known as Span 40.

In one aspect a pharmaceutical composition according to the present invention is a liquid and comprises at least one therapeutic hydrophilic protein or polypeptide, at least one fatty acid acylated amino acid, at least one polyglycerol fatty acid ester, further comprising sorbitan fatty acid ester and a polar or semipolar solvent. In one aspect a pharmaceutical composition according to the present invention is a liquid and comprises a therapeutic hydrophilic protein or polypeptide, at least one fatty acid acylated amino acid, at least one polyglycerol fatty acid ester, further comprising sorbitan fatty acid ester and a polar or semipolar solvent.

In one aspect a pharmaceutical composition according to the present invention is a liquid and comprises at least one therapeutic hydrophilic protein or polypeptide, at least one fatty acid acylated amino acid, at least one polyglycerol fatty acid ester, further comprising sorbitan fatty acid ester and a polar or semipolar solvent, wherein said polar or semipolar solvent is selected from the group consisting of water or propylene glycol.

In one aspect of the invention the pharmaceutical composition comprises at least one therapeutic active peptide or protein. In one aspect at said at least one therapeutic active peptide or protein is a hydrophilic protein.

In one aspect of the present invention the pharmaceutical composition comprises at least one therapeutic active peptide or protein, which has been pH neutralised.

In one aspect of the invention the therapeutical active peptide or protein is dissolved and the pH of the resulting solution is adjusted to a value of the target pH value, which is 1 unit, alternatively 2 units and alternatively 2.5 pH units above or below the pI of the insulin peptide, whereafter said resulting solution is freeze or spray dryed. In one aspect said pH adjustment is performed with a non-volitale acid or base.

In one aspect of the invention the pharmaceutical composition comprises of at least one insulin peptide and at least on FA-Daa. In one aspect of the invention the pharmaceutical composition comprises of at least one peptide or protein and at least on FA-Daa.

In one aspect of the invention the pharmaceutical composition comprises of at least one insulin peptide and at least on FA-Daa and propylene glycol.

In one aspect the amino acid FA-Daa may be used in a liquid or semisolid liquid and surfactant based delivery system. In one aspect the amino acid FA-Daa may be used in a liquid or semisolid liquid and surfactant based delivery system comprising less than 10% (w/w) water. In one aspect the amino acid FA-Daa may be used in a liquid or semisolid liquid and surfactant based delivery system comprising less than 9% (w/w) water. In one aspect the amino acid FA-Daa may be used in a liquid or semisolid liquid and surfactant based delivery system comprising less than 8% (w/w) water. In one aspect the amino acid FA-Daa may be used in a liquid or semisolid liquid and surfactant based delivery system comprising less than 7% (w/w) water. In one aspect the amino acid FA-Daa may be used in a liquid or semisolid liquid and surfactant based delivery system comprising less than 6% (w/w) water. In one aspect the amino acid FA-Daa may be used in a liquid or semisolid liquid and surfactant based delivery system comprising less than 5% (w/w) water. In one aspect the amino acid FA-Daa may be used in a liquid or semisolid liquid and surfactant based delivery system comprising less than 4% (w/w) water. In one aspect the amino acid FA-Daa may be used in a liquid or semisolid liquid and surfactant based delivery system comprising less than 3% (w/w) water. In one aspect the amino acid FA-Daa may be used in a liquid or semisolid liquid and surfactant based delivery system comprising less than 2% (w/w) water. In one aspect the amino acid FA-Daa may be used in a liquid or semisolid liquid and surfactant based delivery system comprising less than 1% (w/w) water. In one aspect the amino acid FA-Daa may be used in a liquid or semisolid liquid and surfactant based delivery system comprising less than 0% (w/w) water.

In one aspect a pharmaceutical composition according to the present invention comprises at least one therapeutic hydrophilic protein or polypeptide, at least one fatty acid acylated amino acid, at least one high HLB surfactant, at least one low HLB co-surfactant and a polar solvent. In one aspect a pharmaceutical composition according to the present invention comprises a therapeutic hydrophilic protein or polypeptide, at least one fatty acid acylated amino acid, at least one high HLB surfactant, at least one low HLB co-surfactant and a polar solvent.

In one aspect a pharmaceutical composition according to the present invention comprises at least one therapeutic hydrophilic protein or polypeptide, at least one fatty acid acylated amino acid, at least two high HLB surfactants, and a polar solvent. In one aspect a pharmaceutical composition according to the present invention comprises a therapeutic hydrophilic protein or polypeptide, at least one fatty acid acylated amino acid, at least two high HLB surfactants, and a polar solvent.

In one aspect the amino acid FA-Daa may be used in a liquid or semisolid liquid and surfactant based delivery system, such as SEDDS, SMEDDS or SNEDDS. In one aspect the amino acid FA-Daa may be used in a solid surfactant based delivery system comprising less than 10% (w/w) water. In one aspect the amino acid FA-Daa may be used in a solid surfactant based delivery system comprising less than 9% (w/w) water. In one aspect the amino acid FA-Daa may be used in a solid surfactant based delivery system comprising less than 8% (w/w) water. In one aspect the amino acid FA-Daa may be used in a solid surfactant based delivery system comprising less than 7% (w/w) water. In one aspect the amino acid FA-Daa may be used in a solid surfactant based delivery system comprising less than 6% (w/w) water. In one aspect the amino acid FA-Daa may be used in a solid surfactant based delivery system comprising less than 6% (w/w) water. In one aspect the amino acid FA-Daa may be used in a solid surfactant based delivery system comprising less than 5% (w/w) water. In one aspect the amino acid FA-Daa may be used in a solid surfactant based delivery system comprising less than 4% (w/w) water. In one aspect the amino acid FA-Daa may be used in a solid surfactant based delivery system comprising less than 3% (w/w) water. In one aspect the amino acid FA-Daa may be used in a solid surfactant based delivery system comprising less than 2% (w/w) water. In one aspect the amino acid FA-Daa may be used in a solid surfactant based delivery system comprising less than 1% (w/w) water. In one aspect the amino acid FA-Daa may be used in a solid surfactant based delivery system comprising less than 0% (w/w) water. In one aspect the amino acid FA-Daa may be used in a solid surfactant based delivery system, such as SEDDS, SMEDDS or SNEDDS.

In one aspect the pharmaceutical composition according to the present invention is a liquid.

In one aspect pharmaceutical composition is a liquid or semisolid SEDDS, SMEDDS or SNEDDS comprising FA-Daa's according to the invention and is encapsulated with any available soft- or hard capsule technology to result in a solid oral pharmaceutical dosage form. In one aspect a soft capsule technology used for encapsulating a composition according to the present invention is gelatine free. In one aspect a gelatine free soft capsule technology as commercially known under the name Vegicaps® from Catalent® is used for encapsulation of the pharmaceutical composition according to the present invention.

In one aspect the pharmaceutical composition a liquid or semisolid SEDDS, SMEDDS or SNEDDS comprising FA-Daa's according to the invention and is encapsulated with any available soft- or hard capsule technology to result in a solid oral pharmaceutical dosage form comprising less than 10% (w/w) water. In one aspect the pharmaceutical composition a liquid or semisolid SEDDS, SMEDDS or SNEDDS comprising FA-Daa's according to the invention and is encapsulated with any available soft- or hard capsule technology to result in a solid oral pharmaceutical dosage form comprising less than 9% (w/w) water In one aspect the pharmaceutical composition a liquid or semisolid SEDDS, SMEDDS or SNEDDS comprising FA-Daa's according to the invention and is encapsulated with any available soft- or hard capsule technology to result in a solid oral pharmaceutical dosage form comprising less than 8% (w/w) water. In one aspect the pharmaceutical composition a liquid or semisolid SEDDS, SMEDDS or SNEDDS comprising FA-Daa's according to the invention and is encapsulated with any available soft- or hard capsule technology to result in a solid oral pharmaceutical dosage form comprising less than 7% (w/w) water In one aspect the pharmaceutical composition a liquid or semisolid SEDDS, SMEDDS or SNEDDS comprising FA-Daa's according to the invention and is encapsulated with any available soft- or hard capsule technology to result in a solid oral pharmaceutical dosage form comprising less than 6% (w/w) water. In one aspect the pharmaceutical composition a liquid or semisolid SEDDS, SMEDDS or SNEDDS comprising FA-Daa's according to the invention and is encapsulated with any available soft- or hard capsule technology to result in a solid oral pharmaceutical dosage form comprising less than 5% (w/w) water. In one aspect the pharmaceutical composition a liquid or semisolid SEDDS, SMEDDS or SNEDDS comprising FA-Daa's according to the invention and is encapsulated with any available soft- or hard capsule technology to result in a solid oral pharmaceutical dosage form comprising less than 4% (w/w) water. In one aspect the pharmaceutical composition a liquid or semisolid SEDDS, SMEDDS or SNEDDS comprising FA-Daa's according to the invention and is encapsulated with any available soft- or hard capsule technology to result in a solid oral pharmaceutical dosage form comprising less than 3% (w/w) water. In one aspect the pharmaceutical composition a liquid or semisolid SEDDS, SMEDDS or SNEDDS comprising FA-Daa's according to the invention and is encapsulated with any available soft- or hard capsule technology to result in a solid oral pharmaceutical dosage form comprising less than 2% (w/w) water. In one aspect the pharmaceutical composition a liquid or semisolid SEDDS, SMEDDS or SNEDDS comprising FA-Daa's according to the invention and is encapsulated with any available soft- or hard capsule technology to result in a solid oral pharmaceutical dosage form comprising less than 1% (w/w) water In one aspect the pharmaceutical composition a liquid or semisolid SEDDS, SMEDDS or SNEDDS comprising FA-Daa's according to the invention and is encapsulated with any available soft- or hard capsule technology to result in a solid oral pharmaceutical dosage form comprising less than 0% (w/w) water.

In one aspect a liquid or semisolid formulation according to the invention is encapsulated with any available soft- or hard capsule technology to result in a solid oral pharmaceutical dosage form further comprising an enteric or delayed release coating.

In one aspect a liquid or semisolid formulation according to the invention is encapsulated with any available enteric soft- or hard capsule technology to result in a solid oral pharmaceutical dosage.

In one aspect a liquid or semisolid SEDDS, SMEDDS or SNEDDS comprising FA-Daa's according to the invention is encapsulated with any available soft- or hard capsule technology to result in a solid oral pharmaceutical dosage form further comprising an enteric or delayed release coatings. In one aspect a liquid or semisolid SEDDS, SMEDDS or SNEDDS comprising FA-Daa's according to the invention is encapsulated with any available enteric soft- or hard capsule technology to result in a solid oral pharmaceutical dosage.

In one aspect a liquid or semisolid SEDDS, SMEDDS or SNEDDS comprising FA-Daa's according to the invention is encapsulated with any available soft- or hard capsule technology to result in a solid oral pharmaceutical dosage form which may further comprise an enteric or delayed release coatings, such as poly(meth)acrylates, commercially known as Eudragit®.

In one aspect, the coating comprises at least one release modifying polymer which can be used to control the site where the drug (insulin derivative) is released. The modified release polymer can be a polymethacrylate polymer such as those sold under the Eudragit® trade name (Evonik Rohm GmbH, Darmstadt, Germany), for example Eudragit® L30 D55, Eudragit® L100-55, Eudragit® L100, Eudragit® S100, Eudragit® S12.5, Eudragit® FS30D, Eudragit® NE30D and mixtures thereof as e.g. described in Eudragit® Application Guidelines, Evonik Industries, 11th edition, September 2009.

In one aspect of the invention the pharmaceutical composition is a formulation, comprising at least one insulin and at least one FA-Daa, propylene glycol.

In one aspect of the invention the pharmaceutical composition comprises of at least one insulin and at least one FA-Daa, propylene glycol.

In one aspect of the invention the pharmaceutical comprises at least one peptide or protein and at least one FA-Daa, propylene glycol.

In one aspect of the invention the pharmaceutical composition is a SEDDS, SMEDDS or SNEDDS, comprising at least one peptide or protein and at least one FA-Daa, propylene glycol.

The components of the drug delivery system may be present in any relative amounts. In one aspect the drug delivery system comprises up to 90% of a surfactant, or up to 90% of a polar organic solvent such as Polyethylene glycol (PEG) 300 g/mol, PEG 400 g/mol, PEG 600 g/mol, PEG 1000 g/mol, or up to 90% of a lipid component. PEGs are prepared by polymerization of ethylene oxide and are commercially available over a wide range of molecular weights from 300 g/mol to Ser. No. 10/000,000 g/mol.

In one aspect the oral pharmaceutical composition comprises from 5 to 20% of propylene glycol.

In one aspect, the oral pharmaceutical composition comprises at least one FA-Daa, propylene glycol, and at least two non ionic surfactants.

In one aspect, the oral pharmaceutical composition comprises at least one FA-Daa, propylene glycol, polysorbate 20 and a co-surfactant. Polysorbate 20 is a polysorbate surfactant whose stability and relative non-toxicity allows it to be used as a detergent and emulsifier in a number of domestic, scientific, and pharmacological applications. The number 20 refers to the total number of oxyethylene —(CH₂CH₂O)— groups found in the molecule.

In one aspect of the present invention, the oral pharmaceutical composition comprises at least one FA-Daa, propylene glycol, polysorbate 20 and a polyglycerol fatty acid ester.

In one aspect, the oral pharmaceutical composition comprises at least one FA-Daa, propylene glycol, polysorbate 20 and a co-surfactant.

In one aspect, the oral pharmaceutical composition comprises at least one FA-Daa, propylene glycol, polysorbate 20 and a polyglycerol fatty acid ester such as diglycerol monocaprylate.

In certain aspects of the present invention, the pharmaceutical composition may comprise additional excipients commonly found in pharmaceutical compositions, examples of such excipients include, but are not limited to, antioxidants, antimicrobial agents, enzyme inhibitors, stabilizers, preservatives, flavors, sweeteners and other components as described in Handbook of Pharmaceutical Excipients, Rowe et al., Eds., 4th Edition, Pharmaceutical Press (2003), which is hereby incorporated by reference

These additional excipients may be in an amount from about 0.05-5% by weight of the total pharmaceutical composition. Antioxidants, anti-microbial agents, enzyme inhibitors, stabilizers or preservatives typically provide up to about 0.05-1% by weight of the total pharmaceutical composition. Sweetening or flavouring agents typically provide up to about 2.5% or 5% by weight of the total pharmaceutical composition.

Oral pharmaceutical compositions according to this invention may be formulated as solid dosage forms.
Oral pharmaceutical compositions according to this invention may be formulated as solid dosage forms and may be selected from the group consisting of capsules, tablets, dragees, pills, lozenges, powders and granules.
Oral pharmaceutical compositions according to this invention may be formulated as multiparticulate dosage forms.
Oral pharmaceutical compositions according to this invention may be formulated as multiparticulate dosage forms and may be selected from the group consisting of pellets, microparticles, nanoparticles, liquid or semisolid fill formulations in soft- or hard capsules, enteric coated soft-hard capsules.
In one aspect the oral pharmaceutical compositions may be prepared with one or more coatings such as enteric coatings or be formulated as delayed release formulations according to methods well known in the art.
Enteric or delayed release coatings according to this invention may be based on poly(meth)acrylates commercially known as Eudragit®.

In one aspect, the pharmaceutical composition according to the invention is used for the preparation of a medicament.

In one aspect, the pharmaceutical composition according to the invention is used for the preparation of a medicament for the treatment or prevention of hyperglycemia, type 2 diabetes mellitus, impaired glucose tolerance, type 1 diabetes mellitus and/or anti obesity treatment.

The terms “fatty acid N-acylated D-amino acid” or “acylated D-amino acid” or “FA-Daa” may be used interchangeable and refer when used herein to a D-amino acids that is acylated with a fatty acid at its alpha-amino group or any corresponding salt thereof. FA-Daa according to the present invention are based on the acidic or non-polar uncharged amino acids selected form the group consisting of: Alanine (Ala), Valine (Val), Leucine (Leu), Leucine (Ile), Phenylalanine (Phe), Tryptophan (Trp), Proline (Pro), Apartic acid (Asp), Gltamic acid (Glu) Tyrosine (Tyr). In one aspect the specific D-amino acids according to the present invention are indicated by adding a D before the name of the amino acid. This is exemplified by the amino acid Valine, wherein the D-amino acid of Valine according to this invention is indicated by the term “D-Valine”.

The term “D-amino acid” as used herein refers to an amino acid with a stereo configuration of the chiral carbon atom in the D-configuration. In the R/S system, the chiral carbon in all D-amino acids is in the (R) configuration with the exception of D-cysteine where the chiral carbon is in (S) configuration.

Amino acids exist in the stereoisomeric form of either D (dextro) or L (levo). The D and L refer to the absolute confirmation of optically active compounds. With the exception of glycine, all other amino acids are mirror images that can not be superimposed. Most of the amino acids found in nature are of the L-type. Hence, eukaryotic proteins are always composed of L-amino acids although D-amino acids are found in bacterial cell walls and in some peptide antibiotics. At least 300 amino acids have been described in nature but only twenty of these are typically found as components in human peptides and proteins. Twenty standards amino acids are used by cells in peptide biosynthesis, and these are specified by the general genetic code. The twenty standard amino acids are Alanine (Ala), Valine (Val), Leucine (Leu), Leucine (Ile), Phenylalanine (Phe), Tryptophan (Trp), Methionine (Met), Proline (Pro), Apartic acid (Asp), Gltamic acid (Glu), Glycine (Gly), Serine (Ser), Threonine (Thr), Cysteine (Cys), Tyrosine (Tyr), Apsaragine (Asn), Glutamine (Gin), Lysine (Lys), Arginine (Arg) and Histidine (His).

In one aspect the amino moiety is in the form of a pure enantiomer. In one aspect the chiral carbon atom in the amino acid moiety is in the D form. In the R/S system, the chiral carbon in all D-amino acids according to the present invention is in the (R) configuration.

The amino acid moiety of the modified FA-Daa may be in the form of a pure (>90%) enantiomer wherein the stereo configuration of the chiral carbon atom in the amino acid moiety is D. The amino acid moiety of the modified FA-Daa may be in the form of a mixture of enantiomers wherein at least 10% of the enantiomers correspond to D-enantiomer. The amino acid moiety of the modified FA-Daa may be in the form of a mixture of enantiomers wherein at least 20% of the enantiomers correspond to D-enantiomer. The amino acid moiety of the modified FA-Daa may be in the form of a mixture of enantiomers wherein at least 30% of the enantiomers correspond to D-enantiomer. The amino acid moiety of the modified FA-Daa may be in the form of a mixture of enantiomers wherein at least 40% of the enantiomers correspond to D-enantiomer. The amino acid moiety of the modified FA-Daa may be in the form of a mixture of enantiomers wherein at least 60% of the enantiomers correspond to D-enantiomer. The amino acid moiety of the modified FA-Daa may be in the form of a mixture of enantiomers wherein at least 70% of the enantiomers correspond to D-enantiomer. The amino acid moiety of the modified FA-Daa may be in the form of a mixture of enantiomers wherein at least 80% of the enantiomers correspond to D-enantiomer.

In one aspect of the invention the amino acid moiety is in the form of a mixture of enantiomers.

The term “fatty acid chain” may be used interchangeably with the term “fatty acid moiety” and refers to a hydrocarbon chain comprising at least one acid group. The term hydrocarbon chain as used herein could be but is not limited to alkane chain with a general formula CₙH₂ₙ₊₂ that is substituted with an acid group typically at one end.

The term “non-polar uncharged amino acids” as used herein refer to categorisation of amino acids used by the person skilled in the art. The term “non-polar uncharged amino acids” as used herein refer to categorisation of amino acids used by the person skilled in the art and may specifically be selected from the group consisting of: Alanine (Ala, A), Leucine (Ile, I), Leucine (Leu, L), Proline (Pro, P), Valine (Val, V).

As used herein the term “acidic amino acid” refers to categorisation of amino acids used by the person skilled in the art. The term “acidic amino acids” as used herein refers to catogorisation of amino acids used by the person skilled in the art and it is understood such that the side chain of this amino acid is negatively charged under physiological conditions (i.e. pH˜7). As used herein the term “acidic amino acid” as used herein refer to categorisation of amino acids used by the person skilled in the art and may specifically be selected from the following amino acids: Aspartic acid (Asp) and Glutamic acid (Glu).

The term “fasted state simulated intestinal fluid” or “FASSIF” as used herin refers to 3 mM sodium taurocholate, 0.75 mM lecithine, 10.5 mM NaOH, 28.65 mM NaH₂PO₄, 105.85 mM NaCl, pH=6.5 and osmomolarity 270±10 mOsmol (http://biorelevant.com/).

The term “fed state simulated intestinal fluid” or “FESSIF” as used herein refers to 15 mM sodium taurocholate, 3.75 mM lecithine, 101.02 mM NaOH, 144.05 mM glacial acetic acid, 203.18 mM NaCl, pH=5 and osmomolarity 635±10 mOsmol (http://biorelevant.com/).

With the term “oral bioavailability” is herein meant the fraction of the administered dose of drug that reaches the systemic circulation after having been administered orally. By definition, when a medication is administered intravenously, its bioavailability is 100%. However, when a drug is administered orally the bioavailability of the active ingredient decreases due to incomplete absorption and first-pass metabolism. The biological activity of an insulin peptide may be measured in an assay as known by a person skilled in the art as e.g. described in WO 2005012347.

The term “surfactant” as used herein refers to any substance, in particular a detergent, that can adsorb at surfaces and interfaces, such as but not limited to liquid to air, liquid to liquid, liquid to container or liquid to any solid. In one aspect the term “surfactant” includes FA-Daa.

The term “permeation enhancer” when used herein refers to biologicals or chemicals that promote the absorption of drugs.

The term “preservative” as used herein refers to a chemical compound which is added to a pharmaceutical composition to prevent or delay microbial activity (growth and metabolism). Examples of pharmaceutically acceptable preservatives are phenol, m-cresol and a mixture of phenol and m-cresol.

The term “macromolecular” or “macromolecule” used herein refer to non-polymeric molecules and comprises nucleic acids, peptides, proteins, carbohydrates, and lipids.

The term “polypeptide” and “peptide” as used herein means a compound composed of at least two constituent amino acids connected by peptide bonds. The constituent amino acids may be from the group of the amino acids encoded by the genetic code and they may be natural amino acids which are not encoded by the genetic code, as well as synthetic amino acids. Commonly known natural amino acids which are not encoded by the genetic code are e.g., γ-carboxyglutamate, ornithine, phosphoserine, D-alanine and D-glutamine. Commonly known synthetic amino acids comprise amino acids manufactured by chemical synthesis, i.e. D-isomers of the amino acids encoded by the genetic code such as D-alanine and D-leucine, Aib (a-aminoisobutyric acid), Abu (a-aminobutyric acid), Tle (tert-butylglycine), β-alanine, 3-aminomethyl benzoic acid, anthranilic acid.

The term “Protein” as used herein means a biochemical compound consisting of one or more polypeptides. The term “hydrophilic peptide or protein” as used herin refers to the overall physical/chemical characteristics of the peptide, protein, analogue or derivative, such as but not limited to the group of insulin, insulin analogues and insulin derivatives. The term “insulin peptide or protein” as used herin refers to insulin, insulin analogues and insulin derivatives. The term “insulin peptide or protein” as used herin includes insulin, insulin analogues and insulin derivatives. The term “hydropilic peptide or protein” as used herin, also refers to the physical/chemical characteristics of the parts of the peptide or protein which has been derivatised, such as but not limited to the insulin backbone (i.e. the insulin subject to derivatisation), that has been derivatised.

The term “macromolecular therapeutic” or “therapeutic macromolecule” may be used interchangeable and as used herein refer to nucleic acids, peptides, proteins, carbohydrates, and lipids as well as non-polymeric molecules with large molecular mass used in therapy and includes without being limited therto insulin, insulin analouges and insulin derivatives. In one aspect lage molecular mass means a molecular mass above 1500 Da. In one aspect large molecular mass means a molecular mass between 150 Da and 6000 Da. In one aspect large molecular mass means a molecular mass between 150 Da and 8000 Da.

The term “drug”, “therapeutic”, “medicament” or “medicine” when used herein refer to an active ingredient used in a pharmaceutical composition, which may be used in therapy and thus also refer to what was defined as “macromolecular therapeutic” or “therapeutic macromolecule” in the present patent application.

With “insulin peptide”, “an insulin peptide” or “the insulin peptide” as used herein is meant human insulin comprising disulfide bridges between CysA7 and CysB7 and between CysA20 and CysB19 and an internal disulfide bridge between CysA6 and CysA11 or an insulin analogue or derivative thereof.

The term “peptide” as used herein comprises also peptides, proteins, conjugates of such peptides and proteins and biologically active fragments thereof. The term “protein” comprises peptides and also refers to proteins and biologically active fragments thereof.

Human insulin consists of two polypeptide chains, the A and B chains which contain 21 and 30 amino acid residues, respectively. The A and B chains are interconnected by two disulphide bridges. Insulin from most other species is similar, but may contain amino acid substitutions in some positions.

The term “insulin” as used herein is, if not specified further, an insulin selected from the group consisting of human insulin, insulin analogues and insulin derivatives.

An insulin analogue as used herein is a polypeptide, such as an insulin peptide which has a molecular structure which formally may be derived from the structure of a naturally occurring insulin, for example that of human insulin, by deleting and/or substituting at least one amino acid residue occurring in the natural insulin and/or by adding at least one amino acid residue.

The term “insulin analogue” as used herein means a modified insulin wherein one or more amino acid residues of the insulin have been substituted by other amino acid residues and/or wherein one or more amino acid residues have been deleted from the insulin and/or wherein one or more amino acid residues have been added and/or inserted to the insulin.

In one aspect an insulin analogue according to the invention comprises less than 8 modifications (substitutions, deletions, additions) relative to human insulin.

In one aspect an insulin analogue comprises less than 7 modifications (substitutions, deletions, additions) relative to human insulin. In one aspect an insulin analogue comprises less than 6 modifications (substitutions, deletions, additions) relative to human insulin.

In one aspect an insulin analogue comprises less than 5 modifications (substitutions, deletions, additions) relative to human insulin. In one aspect an insulin analogue comprises less than 4 modifications (substitutions, deletions, additions) relative to human insulin. In one aspect an insulin analogue comprises less than 3 modifications (substitutions, deletions, additions) relative to human insulin. In one aspect an insulin analogue comprises less than 2 modifications (substitutions, deletions, additions) relative to human insulin.

The term “insulin derivative” as used herein refers to chemically modified parent insulin or an analogue thereof, wherein the modification(s) are in the form of attachment of amides, carbohydrates, alkyl groups, acyl groups, esters, PEGylations, and the like.

An insulin derivative according to the invention is a naturally occurring insulin or an insulin analogue which has been chemically modified, e.g. by introducing a side chain in one or more positions of the insulin backbone or by oxidizing or reducing groups of the amino acid residues in the insulin or by converting a free carboxylic group to an ester group or to an amide group. Other derivatives are obtained by acylating a free amino group or a hydroxy group, such as in the B29 position of human insulin or desB30 human insulin.

Herein, the term “acylated insulin” covers modification of insulin by attachment of one or more lipophilic substituents optionally via a linker to the insulin peptide.

An insulin derivative is thus human insulin, an insulin analogue or insulin peptide which comprises at least one covalent modification such as a side-chain attached to one or more amino acids of the insulin peptide.

Herein, the naming of the insulin peptide is done according to the following principles: The names are given as mutations and modifications (acylations) relative to human insulin. For the naming of the acyl moiety, the naming is done according to IUPAC nomenclature and in other cases as peptide nomenclature. For example, naming the acyl moiety:

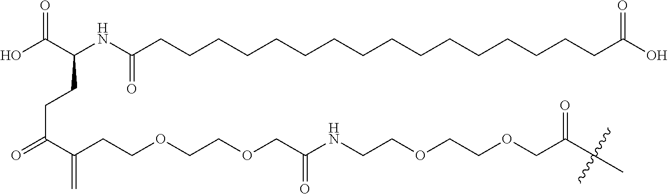 [SMILES: *C(=O)COCCOCCNC(=O)COCCOCCC(=C)C(=O)CC[C@H](NC(=O)CCCCCCCCCCCCCCCCC(=O)O)C(=O)O]

may be e.g. “octadecanedioyl-γ-L-Glu-OEG-OEG”, or “17-carboxyheptadecanoyl-γ-L-Glu-OEG-OEG”, wherein OEG is short hand notation for the amino acid —NH(CH₂)₂O(CH₂)₂OCH₂CO—, and γ-L-Glu (or g-L-Glu) is short hand notation for the L-form of the amino acid gamma glutamic acid moiety.

In one aspect an insulin derivative in an oral pharmaceutical composition according to the invention is an insulin peptide that is acylated in one or more amino acids of the insulin peptide.

In one aspect an insulin derivative in an oral pharmaceutical composition according to the invention is an insulin peptide that is stabilized towards proteolytic degradation (by specific mutations) and further acylated at the B29-lysine. A non-limiting example of insulin peptides that are stabilized towards proteolytic degradation (by specific mutations) may e.g. be found in WO 2008034881, which is hereby incorporated by reference.

The acylated insulin peptides suitable for this invention may be mono-substituted having only one acylation group attached to a lysine amino acid residue in the protease stabilized insulin molecule.

A non-limiting list of acylated insulin peptides suitable for the liquid oral pharmaceutical composition of the invention may e.g. be found in WO 2009115469 such as in the passage beginning on page 24 thereof and continuing the next 6 pages.

In one aspect of the invention, the acylated insulin peptide is selected from the group consisting of:

B29K(N(ε)hexadecanedioyl-γ-L-Glu) A14E B25H desB30 human insulin;

B29K(N(ε)octadecanedioyl-γ-L-Glu-OEG-OEG) desB30 human insulin;

B29K(N(ε)octadecanedioyl-γ-L-Glu) A14E B25H desB30 human insulin;

B29K(N(ε)eicosanedioyl-γ-L-Glu) A14E B25H desB30 human insulin;

B29K(N(ε)octadecanedioyl-γ-L-Glu-OEG-OEG) A14E B25H desB30 human insulin;

B29K(N(ε)eicosanedioyl-γ-L-Glu-OEG-OEG) A14E B25H desB30 human insulin;

B29K(N(ε)eicosanedioyl-γ-L-Glu-OEG-OEG)A14E B16H B25H desB30 human insulin;

B29K(N(ε)hexadecanedioyl-γ-L-Glu)A14E B16H B25H desB30 human insulin;

B29K(N(ε)eicosanedioyl-γ-L-Glu-OEG-OEG)A14E B16H B25H desB30 human insulin; and

B29K(N(ε)octadecanedioyl)A14E B25H desB30 human insulin.

B29K(N(ε)hexadecanedioyl-γ-L-Glu) A14E B25H desB27 desB30 human insulin;

B29K(N(ε)octadecanedioyl-γ-L-Glu-OEG-OEG) desB27 desB30 human insulin;

B29K(N(ε)octadecanedioyl-γ-L-Glu) A14E B25H desB27 desB30 human insulin;

B29K(N(ε)eicosanedioyl-γ-L-Glu) A14E B25H desB27 desB30 human insulin;

B29K(N(ε)octadecanedioyl-γ-L-Glu-OEG-OEG) A14E B25H desB27 desB30 human insulin;

B29K(N(ε)eicosanedioyl-γ-L-Glu-OEG-OEG) A14E B25H desB27 desB30 human insulin;

B29K(N(ε)eicosanedioyl-γ-L-Glu-OEG-OEG) A14E B16H B25H desB27 desB30 human insulin;

B29K(N(ε)hexadecanedioyl-γ-L-Glu) A14E B16H B25H desB27 desB30 human insulin;

B29K(N(ε)eicosanedioyl-γ-L-Glu-OEG-OEG) A14E B16H B25H desB27 desB30 human insulin; and

B29K(N(ε)octadecanedioyl) A14E B25H desB27 desB30 human insulin.

In one aspect of the invention, the insulin derivative is B29K(N(ε)octadecanedioyl-γ-L-Glu-OEG-OEG) A14E B25H desB30 human insulin.

A non-limiting list of acylated insulin peptides suitable for the liquid oral pharmaceutical composition of the invention may e.g. be found in the PCT application WO2011068019 such as outlined and exemplified in but not limited to the passage beginning on page 20 line 20 and continuing the next 6 pages, to be publwashed in April 2013.

In one aspect of the invention, the acylated insulin peptide is selected from the group consisting of N-terminally modified insulin consisting of:

A1(N^{α},N^{α}-Dimethyl), A14E, B1(N^{α},N^{α}-dimethyl), B25H, B29K(N^{ε}octadecanedioyl-gGlu-2xOEG), desB30 human insulin

A1(N^{α},N^{α}-Diethyl), A14E, B1(N^{α},N^{α}-diethyl), B25H, B29K(N^{ε}Octadecanedioyl-gGlu-2xOEG), desB30 human insulin

A1(N^{α},N^{α}-Dimethyl), A14E, B1(N^{α},N^{α}-dimethyl), B16H, B25H, B29K(N^{ε}hexadecanedioyl-gGlu), desB30 human insulin

A1(N^{α},N^{α}-Dimethyl), A14E, B1(N^{α},N^{α}-dimethyl), B25H, desB27, B29K(N^{ε}octadecanedioyl-gGlu), desB30 human insulin

A1(N^{α},N^{α}-Dimethyl), A14E, B1(N^{α},N^{α}-dimethyl), B25H, desB27, B29K(N^{ε}octadecanedioyl-gGlu-2xOEG), desB30 human insulin

A1(N^{α},N^{α}-Dimethyl), A14E, B1(N^{α},N^{α}-dimethyl), desB27, B29K(N^{ε}octadecanedioyl-gGlu-2xOEG), desB30 human insulin

A1(N^{α},N^{α}-Dimethyl), A14E, B1(N^{α},N^{α}-dimethyl), B16H, B25H, B29K(N^{ε}eicosanedioyl-gGlu-2xOEG), desB30 human insulin

A1G(N^{α},N^{α}-Dimethyl), A14E, B1F(N^{α},N^{α}-dimethyl), B25H, desB27, B29K(N^{ε}hexadecanedioyl-gGlu), desB30 human insulin

A1G(N^{α},N^{α}-Dimethyl), A14E, B1F(N(alpha),N(N^{α},N^{α}-dimethyl), B25H, desB27, B29K(N^{ε}hexadecanedioyl-gGlu-2xOEG), desB30 human insulin

A1(N^{α},N^{α}-Dimethyl), A14E, B1(N^{α},N^{α}-dimethyl), desB27, B29K(N^{ε}octadecanedioyl-gGlu), desB30 human insulin

A1(N^{α},N^{α}-Dimethyl), A14E, B1(N^{α},N^{α}-dimethyl), B25H, B29K(N^{ε}octadecanedioyl-gGlu), desB30 human insulin

A1(N^{α}Carbamoyl), A14E, B1(N^{α}Carbamoyl), B25H, B29K(N^{ε}octadecanedioyl-gGlu-2xOEG), desB30 human insulin

A1(N^{α}Carbamoyl), A14E, B1(N^{α}Carbamoyl), B25H, B29K(N^{ε}hexadecanedioyl-gGlu), desB30 human insulin

A1(N^{α}Carbamoyl), A14E, B1(N^{α}Carbamoyl), B25H, B29K(N^{ε}eicosanedioyl-gGlu), desB30 human insulin

A1(N^{α}Carbamoyl), A14E, B1(N^{α}Carbamoyl), B25H, B29K(N^{ε}eicosanedioyl-gGlu-2xOEG), desB30 human insulin

A1(N^{α}Carbamoyl), A14E, B1(N^{α}Carbamoyl), B16H, B25H, B29K(N^{ε}eicosanedioyl-gGlu-2xOEG), desB30 human insulin

A1(N^{α}Carbamoyl), A14E, B1(N^{α}Carbamoyl), B25H, desB27, B29K(N^{ε}octadecandioyl-gGlu), desB30 human insulin

A1(N^{α}Carbamoyl), A14E, B1(N^{α}Carbamoyl), B25H, desB27, B29K(N^{ε}octadecandioyl-gGlu-2xOEG), desB30 human insulin

A1G(N(alpha)carbamoyl), A14E, B1F(N(alpha)carbamoyl), desB27, B29K(N(eps)hexadecanedioyl-gGlu), desB30 human insulin

A1G(N(alpha)carbamoyl), A14E, B1F(N(alpha)carbamoyl), desB27, B29K(Neps)-hexadecanedioyl-gGlu-2xOEG), desB30 human insulin

A1G(N(alpha)carbamoyl), A14E, B1F(N(alpha)carbamoyl), desB27, B29K(Neps)-eicosanedioyl-gGlu), desB30 human insulin

A1G(N^{α}carbamoyl), A14E, B1F(N^{α}carbamoyl), B16H, desB27, B29K(Neps)-eicosanedioyl-gGlu-2xOEG), desB30 human insulin

A1(N^{α}Carbamoyl), A14E, B1(N^{α}Carbamoyl), desB27, B29K(N^{ε}octadecanedioyl-gGlu), desB30 human insulin

A1(N^{α}Carbamoyl), A14E, B1(N^{α}Carbamoyl), B16H, B25H, B29K(N^{ε}eicosanedioyl-gGlu), desB30 human insulin

A1(N^{α}Carbamoyl), A14E, B1(N^{α}Carbamoyl), desB27, B29K(N^{ε}octadecanedioyl-gGlu-2xOEG), desB30 human insulin

A1(N^{α}Carbamoyl), A14E, B1(N^{α}carbamoyl), B25H, B29K(N^{ε}octadecanedioyl-gGlu), desB30 human insulin

A1(N^{α}Carbamoyl), A14E, B1(N^{α}Carbamoyl), B16H, B25H, B29K(N^{ε}eicosanedioyl-gGlu), desB30 human insulin

A1G(N^{α}carbamoyl), A14E, B1F(N^{α}carbamoyl), B25H, desB27, B29K(N^{ε}eicosanedioyl-gGlu-2xOEG), desB30 human insulin

A1G(N^{α}carbamoyl), A14E, B1F(N^{α}carbamoyl), desB27, B29K(N^{ε}eicosanedioyl-gGlu-2xOEG), desB30 human insulin

A1G(N^{α}carbamoyl), A14E, B1F(N^{α}carbamoyl), B16H, desB27, B29K(N^{ε}-eicosanedioyl-gGlu-2xOEG), desB30 human insulin

A1G(N^{α}thiocarbamoyl), A14E, B1F(N N^{α}thiocarbamoyl), B25H, desB27, B29K(N^{ε}-octadecanedioyl-gGlu-2xOEG), desB30 human insulin

A1(N^{α}Acetyl), A14E, B1(N^{α}Acetyl), B25H, B29K(N^{ε}hexadecanedioyl-gGlu), desB30 human insulin

A1(N^{α}Acetyl), A14E, B1(N^{α}Acetyl), B25H, desB27, B29K(N^{ε}octadecanedioyl-gGlu), desB30 human insulin

A1(N^{α}Acetyl), A14E, B1(N^{α}Acetyl), B25H, B29K(N^{ε}octadecandioyl-gGlu-2xOEG), desB30 human insulin

A1(N^{α}Dimethylglycyl), A14E, B1(N^{α}Dimethylglycyl), B25H, B29K(N^{ε}octadecanedioyl-gGlu-2xOEG), desB30 human insulin

A1(N^{α}3-(N,N-Dimethylamino)propionyl), A14E, B1 (N^{α}3-(N,N-dimethylamino)propionyl), B25H, B29K(N^{ε}octadecanedioyl-gGlu-2xOEG), desB30 human insulin

A1(N^{α}4-(N,N-Dimethylamino)butanoyl), A14E, B1 (N^{α}4-(N,N-dimethylamino)butanoyl), B25H, B29K(N^{ε}octadecanedioyl-gGlu-2xOEG), desB30 human insulin

A1(N^{α}3-(1-Piperidinyl)propionyl), A14E, B1(N^{α}3-(1-piperidinyl)propionyl), B25H, B29K(N^{ε}octadecanedioyl-gGlu-2xOEG), desB30 human insulin

A1(N^{α}Dimethylglycyl), A14E, B1(N^{α}Dimethylglycyl), B25H, desB27, B29K(N^{ε}octadecanedioyl-gGlu), desB30 human insulin

A1G(N^{α}acetyl), A14E, B1F(N^{α}acetyl),B25H, desB27, B29K(N^{ε}octadecanedioyl-gGlu-2xOEG), desB30 human insulin

A1G(N^{α}2-Picolyl), A14E, B1F(N^{α}2-Picolyl), B25H, desB27, B29K(N(eps)-octadecanedioyl-gGlu-2xOEG), desB30 human insulin

A1(N^{α}Acetyl), A14E, B1(N^{α}Acetyl), B25H, B29K(N^{ε}eicosanedioyl-gGlu), desB30 human insulin

A1(N^{α}Acetyl), A14E, B1(N^{α}Acetyl), B25H, B29K(N^{ε}eicosanedioyl-gGlu-2xOEG), desB30 human insulin

A1(N^{α}Acetyl), A14E, B1(N^{α}Acetyl), B16H, B25H, B29K(N^{ε}eicosanedioyl-gGlu-2xOEG), desB30 human insulin

A1(N^{α}Acetyl), A14E, B1(N^{α}Acetyl), B16H, B25H, B29K(N^{ε}eicosanedioyl-gGlu), desB30 human insulin

A1(N^{α}Dimethylglycyl), A14E, B1(N^{α}Dimethylglycyl), B16H, B25H, B29K(N^{ε}hexadecanedioyl-gGlu), desB30 human insulin

A-1(N^{α}Trimethyl), A14E, B-1(N^{α}Trimethyl), B25H, B29K(N^{ε}octadecanedioyl-gGlu-2xOEG), desB30 human insulin

A1(N^{α}Acetyl), A14E, B1(N^{α}Acetyl), desB27, B29K(N^{ε}octadecanedioyl-gGlu), desB30 human insulin

A1(N^{α}Acetyl), A14E, B1(N^{α}Acetyl), desB27, B29K(N^{ε}octadecanedioyl-gGlu-2xOEG), desB30 human insulin

A1(N^{α}Acetyl), A14E, B1(N^{α}Acetyl), B25H, B29K(N^{ε}octadecanedioyl-gGlu), desB30 human insulin

A1G(N^{α}Acetyl), A14E, B1F(N^{α}Acetyl), desB27, B29K(N^{ε}eicosanedioyl-gGlu), desB30 human insulin

A1G(N^{α}Acetyl), A14E, B1F(N^{α}Acetyl), desB27, B29K(N^{ε}eicosanedioyl-gGlu-2xOEG), desB30 human insulin

A1G(N^{α}Acetyl), A14E, B1F(N^{α}Acetyl), B25H, desB27, B29K(N^{ε}eicosanedioyl-gGlu-2xOEG), desB30 human insulin

A1(N^{α}Succinyl), A14E, B1(N^{α}succinyl), B25H, desB27, B29K(N^{ε}octadecanedioyl-gGlu-2xOEG), desB30 human insulin

A1(N^{α}Succinyl), A14E, B1(N^{α}succinyl), B25H, B29K(N^{ε}octadecanedioyl-gGlu-2xOEG), desB30 human insulin

A1(N^{α}Succinyl), A14E, B1(N^{α}succinyl), desB27, B29K(N^{ε}octadecanedioyl-gGlu-2xOEG), desB30 human insulin

A1(N^{α}Glutaryl), A14E, B1(N^{α}glutaryl), B25H, B29K(N^{ε}octadecanedioyl-gGlu-2xOEG), desB30 human insulin

A1(N^{α}Glutaryl), A14E, B1(N^{α}glutaryl), desB27, B29K(N^{ε}octadecanedioyl-gGlu-2xOEG), desB30 human insulin

A1(N^{α}Diglycolyl), A14E, B1(N^{α}diglycolyl), B25H, desB27, B29K(N^{ε}octadecanedioyl-gGlu-2xOEG), desB30 human insulin

A1(N^{α}Glutaryl), A14E, B1(N^{α}glutaryl), B25H, desB27, B29K(N^{ε}octadecanedioyl-gGlu-2xOEG), desB30 human insulin

A1(N^{α}Succinyl), A14E, B1(N^{α}succinyl), desB27, B29K(N^{ε}octadecanedioyl-gGlu), desB30 human insulin

A1(N^{α}Succinyl), A14E, B1(N^{α}succinyl), B25H, desB27, B29K(N^{ε}eicosanedioyl-gGlu-2xOEG), desB30 human insulin

A1(N^{α}Succinyl), A14E, B1(N^{α}succinyl), desB27, B29K(N^{ε}eicosanedioyl-gGlu-2xOEG), desB30 human insulin

A1(N^{α}Succinyl), A14E, B1(N^{α}succinyl), B16H, desB27, B29K(N^{ε}eicosanedioyl-gGlu-2xOEG), desB30 human insulin

A1(N^{α}Succinyl), A14E, B1(N^{α}succinyl), B25H, B29K(N^{ε}eicosanedioyl-gGlu-2xOEG), desB30 human insulin

A1(N^{α}Succinyl), A14E, B1(N^{α}succinyl), desB27, B29K(N^{ε}eicosanedioyl-gGlu), desB30 human insulin

A1(N^{α}Glutaryl), A14E, B1(N^{α}glutaryl), desB27, B29K(N^{ε}eicosanedioyl-gGlu), desB30 human insulin

A1(N^{α}Glutaryl), A14E, B1(N^{α}glutaryl), desB27, B29K(N^{ε}eicosanedioyl-gGlu-2xOEG), desB30 human insulin

A1(N^{α}Glutaryl), A14E, B1(N^{α}glutaryl), B25H, desB27, B29K(N^{ε}eicosanedioyl-gGlu-2xOEG), desB30 human insulin

A1(N^{α}Glutaryl), A14E, B1(N^{α}glutaryl), desB27, B29K(N^{ε}eicosanedioyl-gGlu-2xOEG), desB30 human insulin

A1(N^{α}Glutaryl), A14E, B1(N^{α}glutaryl), B25H, B29K(N^{ε}eicosanedioyl-gGlu-2xOEG), desB30 human insulin

In one aspect, an N-terminally modified insulin according to the invention has a peptide part which is selected from the group consisting of the following insulin peptides (i.e. insulins of the invention without N-terminal modifications and without the “lipophilic substituent” or acyl moiety): A14E, B25H, desB30 human insulin; A14H, B25H, desB30 human insulin; A14E, B1E, B25H, desB30 human insulin; A14E, B16E, B25H, desB30 human insulin; A14E, B25H, B28D, desB30 human insulin; A14E, B25H, B27E, desB30 human insulin; A14E, B1E, B25H, B27E, desB30 human insulin; A14E, B1E, B16E, B25H, B27E, desB30 human insulin; A8H, A14E, B25H, desB30 human insulin; A8H, A14E, B25H, B27E, desB30 human insulin; A8H, A14E, B1E, B25H, desB30 human insulin; A8H, A14E, B1E, B25H, B27E, desB30 human insulin; A8H, A14E, B1E, B16E, B25H, B27E, desB30 human insulin; A8H, A14E, B16E, B25H, desB30 human insulin; A14E, B25H, B26D, desB30 human insulin; A14E, B1E, B27E, desB30 human insulin; A14E, B27E, desB30 human insulin; A14E, B28D, desB30 human insulin; A14E, B28E, desB30 human insulin; A14E, B1E, B28E, desB30 human insulin; A14E, B1E, B27E, B28E, desB30 human insulin; A14E, B1E, B25H, B28E, desB30 human insulin; A14E, B1E, B25H, B27E, B28E, desB30 human insulin; A14D, B25H, desB30 human insulin; B25N, B27E, desB30 human insulin; A8H, B25N, B27E, desB30 human insulin; A14E, B27E, B28E, desB30 human insulin; A14E, B25H, B28E, desB30 human insulin; B25H, B27E, desB30 human insulin; B1E, B25H, B27E, desb30 human insulin; A8H, B1E, B25H, B27E, desB30 human insulin; A8H, B25H, B27E, desB30 human insulin; B25N, B27D, desB30 human insulin; A8H, B25N, B27D, desB30 human insulin; B25H, B27D, desB309 human insulin; A8H, B25H, B27D, desB30 human insulin; A(−1)P, A(0)P, A14E, B25H, desB30 human insulin; A14E, B(−1)P, B(0)P, B25H, desB30 human insulin; A(−1)P, A(0)P, A14E, B(−1)P, B(0)P, B25H, desB30 human insulin; A14E, B25H, B30T, B31L, B32E human insulin; A14E, B25H human insulin; A14E, B16H, B25H, desB30 human insulin; A14E, B10P, B25H, desB30 human insulin; A14E, B10E, B25H, desB30 human insulin; A14E, B4E, B25H, desB30 human insulin; A14H, B16H, B25H, desB30 human insulin; A14H, B10E, B25H, desB30 human insulin; A13H, A14E, B10E, B25H, desB30 human insulin; A13H, A14E, B25H, desB30 human insulin; A14E, A18Q, B3Q, B25H, desB30 human insulin; A14E, B24H, B25H, desB30 human insulin; A14E, B25H, B26G, B27G, B28G, desB30 human insulin; A14E, A21G, B25H, B26G, B27G, B28G, desB30 human insulin; A14E, A18Q, A21Q, B3Q, B25H, desB30 human insulin; A14E, A18Q, A21Q, B3Q, B25H, B27E, desB30 human insulin; A14E, A18Q, B3Q, B25H, desB30 human insulin; A13H, A14E, B1E, B25H, desB30 human insulin; A13N, A14E, B25H, desB30 human insulin; A13N, A14E, B1E, B25H, desB30 human insulin; A(−2)G, A(−1)P, A(0)P, A14E, B25H, desB30 human insulin; A14E, B(−2)G, B(−1)P, B(0)P, B25H, desB30 human insulin; A(−2)G, A(−1)P, A(0)P, A14E, B(−2)G, B(−1)P, B(0)P, B25H, desB30 human insulin; A14E, B27R, B28D, B29K, desB30 human insulin; A14E, B25H, B27R, B28D, B29K, desB30 human insulin; A14E, B25H, B26T, B27R, B28D, B29K, desB30 human insulin; A14E, B25H, B27R, desB30 human insulin; A14E, B25H, B27H, desB30 human insulin; A14E, A18Q, B3Q, B25H, desB30 human insulin; A13E, A14E, B25H, desB30 human insulin; A12E, A14E, B25H, desB30 human insulin; A15E, A14E, B25H, desB30 human insulin; A13E, B25H, desB30 human insulin; A12E, B25H, desB30 human insulin; A15E, B25H, desB30 human insulin; A14E, B25H, desB27, desB30 human insulin; A14E, desB27, desB30 human insulin; A14H, desB27, desB30 human insulin; A14E, B16H, desB27, desB30 human insulin; A14H, B16H, desB27, desB30 human insulin; A14E, B25H, B26D, B27E, desB30 human insulin; A14E, B25H, B27R, desB30 human insulin; A14E, B25H, B27N, desB30 human insulin; A14E, B25H, B27D, desB30 human insulin; A14E, B25H, B27Q, desB30 human insulin; A14E, B25H, B27E, desB30 human insulin; A14E, B25H, B27G, desB30 human insulin; A14E, B25H, B27H, desB30 human insulin; A14E, B25H, B27K, desB30 human insulin; A14E, B25H, B27P, desB30 human insulin; A14E, B25H, B27S, desB30 human insulin; A14E, B25H, B27T, desB30 human insulin; A13R, A14E, B25H, desB30 human insulin; A13N, A14E, B25H, desB30 human insulin; A13D, A14E, B25H, desB30 human insulin; A13Q, A14E, B25H, desB30 human insulin; A13E, A14E, B25H, desB30 human insulin; A13G, A14E, B25H, desB30 human insulin; A13H, A14E, B25H, desB30 human insulin; A13K, A14E, B25H, desB30 human insulin; A13P, A14E, B25H, desB30 human insulin; A13S, A14E, B25H, desB30 human insulin; A13T, A14E, B25H, desB30 human insulin; A14E, B16R, B25H, desB30 human insulin; A14E, B16D, B25H, desB30 human insulin; A14E, B16Q, B25H, desB30 human insulin; A14E, B16E, B25H, desB30 human insulin; A14E, B16H, B25H, desB30 human insulin; A14R, B25H, desB30 human insulin; A14N, B25H, desB30 human insulin; A14D, B25H, desB30 human insulin; A14Q, B25H, desB30 human insulin; A14E, B25H, desB30 human insulin; A14G, B25H, desB30 human insulin; A14H, B25H, desB30 human insulin; A8H, B10D, B25H human insulin; and A8H, A14E, B10E, B25H, desB30 human insulin and this aspect may, optionally, comprise B25H, desB30 human insulin and B25N, desB30 human insulin.

In a preferred aspect, a N-terminally modified insulin according to the invention has a peptide part which is selected from the group consisting of: A14E, B25H, desB30 human insulin; A14E, B16H, B25H, desB30 human insulin; A14E, B16E, B25H, desB30 human insulin; A14E, desB27, desB30 human insulin; A14E, B16H, desB27, desB30 human insulin; A14E, B25H, B26G, B27G, B28G, desB30 human insulin; B25H, desB30 human insulin and A14E, B25H, desB27, desB30 human insulin.

In a preferred aspect, a N-terminally modified insulin according to the invention has a peptide part which is selected from any one of the insulins mentioned above that, in addition, are containing the desB27 mutation.

In a preferred aspect, a N-terminally modified insulin according to the invention has a peptide part which is selected from the group consisting of: A14E, B25H, desB27, desB30 human insulin; A14E, B16H, B25H, desB27, desB30 human insulin; A14E, desB27, desB30 human insulin; A14E, B16E, B25H, desB27, desB30 human insulin; and B25H, desB27, desB30 human insulin.

In one aspect, a N-terminally modified insulin according to the invention has a peptide part which is selected from any one of the above mentioned insulins and, in addition, comprise one or two of the following mutations in position A21 and/or B3 to improve chemical stability: A21G, desA21, B3Q, or B3G.

In a preferred aspect, a N-terminally modified insulin according to the invention has a peptide part which is selected from the group consisting of: A14E, A21G, B25H, desB30 human insulin; A14E, A21G, B16H, B25H, desB30 human insulin; A14E, A21G, B16E, B25H, desB30 human insulin; A14E, A21G, B25H, desB27, desB30 human insulin; A14E, A21G, B25H, desB27, desB30 human insulin; A14E, A21G, B25H, B26G, B27G, B28G, desB30 human insulin; A21G, B25H, desB30 human insulin and A21G, B25N, desB30 human insulin, and, preferably, it is selected from the following protease stabilised insulins: A14E, A21G, B25H, desB30 human insulin; A14E, A21G, desB27, desB30 human insulin; A14E, A21G, B16H, B25H, desB30 human insulin; A14E, A21G, B16E, B25H, desB30 human insulin; A14E, A21G, B25H, desB27, desB30 human insulin; A14E, A21G, B25H, desB27, desB30 human insulin; A21G, B25H, desB30 human insulin and A21G, B25N, desB30 human insulin.

Herein, the term “acylated insulin” covers modification of insulin by attachment of one or more lipophilic substituents optionally via a linker to the insulin peptide.

A “lipophilic substituent” is herein understood as a side chain consisting of a fatty acid or a fatty diacid attached to the insulin, optionally via a linker, in an amino acid position such as LysB29, or equivalent.

The insulin peptide may be present in an amount of a pharmaceutical composition according to the invention in up to about 20% such as up to about 10% by weight of the total pharmaceutical composition, or from about 0.1% such as from about 1%. In one aspect of the invention, the insulin peptide is present in an amount from about 0.1% to about 20%, in a further aspect from about 0.1% to 15%, 0.1% to 10%, 1% to 8% or from about 1% to 5% by weight of the total composition. It is intended, however, that the choice of a particular level of insulin peptide will be made in accordance with factors well-known in the pharmaceutical arts, including the solubility of the insulin peptide in the polar organic solvent or optional hydrophilic component or surfactant used, or a mixture thereof, mode of administration and the size and condition of the patient.

Each unit dosage will suitably contain from 1 mg to 200 mg insulin peptide, e.g. about 1 mg, 5 mg, 10 mg, 15 mg, 25 mg, 50 mg, 80 mg, 90 mg, 100 mg, 150 mg, 200 mg insulin peptide, e.g. between 5 mg and 200 mg of insulin peptide. In one aspect of the invention each unit dosage contains between 10 mg and 200 mg of insulin peptide. In a further aspect a unit dosage form contains between 10 mg and 100 mg of insulin peptide.

One aspect of the invention, the unit dosage form contains between 20 mg and 80 mg of insulin peptide. In yet a further aspect of the invention, the unit dosage form contains between 30 mg and 60 mg of insulin peptide.

In one aspect of the invention, the unit dosage form contains between 30 mg and 50 mg of insulin peptide. Such unit dosage forms are suitable for administration 1-5 times daily depending upon the particular purpose of therapy.

The production of polypeptides and peptides such as insulin is well known in the art. Polypeptides or peptides may for instance be produced by classical peptide synthesis, e.g. solid phase peptide synthesis using t-Boc or Fmoc chemistry or other well established techniques, see e.g. Greene and Wuts, “Protective Groups in Organic Synthesis”, John Wiley & Sons, 1999. The polypeptides or peptides may also be produced by a method which comprises culturing a host cell containing a DNA sequence encoding the (poly)peptide and capable of expressing the (poly)peptide in a suitable nutrient medium under conditions permitting the expression of the peptide. For (poly)peptides comprising non-natural amino acid residues, the recombinant cell should be modified such that the non-natural amino acids are incorporated into the (poly)peptide, for instance by use of tRNA mutants.

As used herein, the term “microemulsion preconcentrate” means a composition, which spontaneously forms a microemulsion or a nanoemulsion, e.g., an oil-in-water microemulsion or nanoemulsion, swollen micelle, micellar solution, in an aqueous medium, e.g. in water or in the gastrointestinal fluids after oral application. The composition self-emulsifies upon dilution in an aqueous medium for example in a dilution of 1:5, 1:10, 1:50, 1:100 or higher. In one aspect the composition according to the present invention forms the microemuslion or nanoemulsion comprising particles or domains of a size below 100 nm in diameter. The term “domain size” or “particle size” as used herein refers to repetitive scattering units and may be measured by e.g., small angle Xy-ray. In one aspect of the invention, the domain size is smaller than 150 nm, in another aspect, smaller than 100 nm and in another aspect, smaller than 50 nm, in another aspect, smaller than 20 nm, in another aspect, smaller than 15 nm, in yet another aspect, smaller than 10 nm.

“SEDDS” (self emulsifying drug delivery systems) are herein defined as mixtures of a hydrophilic component, a surfactant, optionally a co-surfactant or lipid component and a therapeutic peptide or protein that forms spontaneously a fine oil in water emulsion when exposed to aqueous media under conditions of gentle agitation or digestive motility that would be encountered in the GI tract. “SMEDDS” (self micro-emulsifying drug delivery systems) are herein defined as isotropic mixtures of a hydrophilic component a surfactant, optionally a co-surfactant or lipid component and a therapeutic peptide or protein that rapidly form an oil in water microemulsion or nanoemulsion when exposed to aqueous media under conditions of gentle agitation or digestive motility that would be encountered in the GI tract. “SNEDDS” (self nano-emulsifying drug delivery systems) are herein defined as isotropic mixtures of a hydrophilic component, at least one surfactant with HLB above 10, optionally a co-surfactant and optionally a lipid component and a therapeutic peptide or protein that rapidly form a nanoemulsion (droplet size below 20 nm in diameter as e.g. measured by PCS) when exposed to aqueous media under conditions of gentle agitation or digestive motility that would be encountered in the GI tract.

As used herein, the term “emulsion” refers to a slightly opaque, opalescent or opague colloidal coarse dispersion that is formed spontaneously or substantially spontaneously when its components are brought into contact with an aqueous medium.

In some aspects the term “microemulsion” refers to a clear or translucent, slightly opaque, opalescent, non-opaque or substantially non-opaque colloidal dispersion that is formed spontaneously or substantially spontaneously when its components are brought into contact with an aqueous medium; a microemulsion is thermodynamically stable and contains homogenously dispersed particles or domains, for example of a solid or liquid state (e.g., liquid lipid particles or droplets), of a mean diameter of less than 150 nm as measured by standard light scattering techniques, e.g. using a MALVERN ZETASIZER Nano ZS. In some aspects when the composition is brought into contact with an aqueous medium a microemulsion is formed which contains homogenously dispersed particles or domains of a mean diameter of less than 100 nm, such as less than 50 nm, less than 40 nm and less than 30 nm. In some aspects “domain” refers to an area of a composition with predominantly lipophilic or hydrophilic composition and said domain may be spherical or have other shapes, such as rod-like or oval. As used herein, the term “nanoemulsion” refers to a clear or translucent, slightly opaque, opalescent, non-opaque or substantially non-opaque colloidal dispersion with particle or droplet size (i.e. domain size) below 20 nm in diameter (as e.g. measured by PCS) that is formed spontaneously or substantially spontaneously when its components are brought into contact with an aqueous medium. In some aspects when the composition is brought into contact with an aqueous medium a nanoemulsion is formed which contains homogenously dispersed particles or domains of a mean diameter of less than 20 nm, such as less than 15 nm, less than 10 nm. In some aspects when the composition is brought into contact with an aqueous medium a nanoemulsion is formed which contains homogenously dispersed particles or domains of a mean diameter of less than 20 nm, such as less than 15 nm, less than 10 nm, and optionally greater than about 2-4 nm. The SEDDS, SMEDDS or SNEDDS self-emulsifies upon dilution in an aqueous medium for example in a dilution of 1:5, 1:10, 1:50, 1:100 or higher. In some aspects the composition forms the microemuslion or nanoemulsion comprising particles or domains of a size below 100 nm in diameter. In some aspects the term “domain size” or “particle size” or “droplet size” as used herein refers to repetitive scattering units and may be measured by e.g., small angle X-ray. In some aspects the domain size is less than 150 nm, such as less than 100 nm or less than 50 nm. In some aspects the domain size is less than 20 nm, such as less than 15 nm or less than 10 nm.

The term “domain size” as used herein refers to repetitive scattering units and may be measured by e.g., small angle Xy-ray. In one aspect of the invention, the domain size is smaller than 150 nm. In one aspect, smaller than 100 nm and In one aspect, smaller than 50 nm, In one aspect, smaller than 20 nm, In one aspect, smaller than 15 nm, in yet another aspect, smaller than 10 nm.

As used herein, the term “nanoemulsion” refers to a clear or translucent, slightly opaque, opalescent, non-opaque or substantially non-opaque colloidal dispersion with particle or droplet size below 20 nm in diameter (as e.g. measured by PCS) that is formed spontaneously or substantially spontaneously when its components are brought into contact with an aqueous medium. In one aspect when the pharmaceutical composition according to the invention is brought into contact with an aqueous medium a microemulsion is formed which contains homogenously dispersed particles or domains of a mean diameter of less than 20 nm, such as less than 15 nm, less than 10 nm and greater than about 2-4 nm.

As used herein the term “spontaneously dispersible” when referring to a preconcentrate refers to a composition that is capable of producing colloidal structures such as nanoemulsions, microemulsions, emulsions and other colloidal systems, when diluted with an aqueous medium when the components of the composition of the invention are brought into contact with an aqueous medium, e.g. by simple shaking by hand for a short period of time, for example for ten seconds. In one aspect a spontaneously dispersible concentrate according to the invention is a SEDDS, SMEDDS or SNEDDS.

The term “non-ionic surfactant” as used herein refers to any substance, in particular a detergent, that can adsorb at surfaces and interfaces, like liquid to air, liquid to liquid, liquid to container or liquid to any solid and which has no charged groups in its hydrophilic group(s) (sometimes referred to as “heads”). The non-ionic surfactant may be selected from a detergent such as ethoxylated castor oil, polyglycolyzed glycerides, acetylated monoglycerides and sorbitan fatty acid esters, polysorbate such as polysorbate-20, polysorbate-40, polysorbate-60, polysorbate-80, super refined polysorbate 20, super refined polysorbate 40, super refined polysorbate 60 and super refined polysorbate 80 (where the term “super refined” is used by the supplier Croda for their high purity Tween products), poloxamers such as poloxamer 124, poloxamer 188 and poloxamer 407, polyoxyethylene sorbitan fatty acid esters, polyoxyethylene derivatives such as alkylated and alkoxylated derivatives (Tweens, e.g. Tween-20 or Tween-80), block copolymers such as polyethyleneoxide/polypropyleneoxide block copolymers (e.g. Pluronics/Tetronics, Triton Xy-100 and/or Synperonic PE/L 44 PEL) and ethoxylated sorbitan alkanoates surfactants (e. g. Tween-20, Tween-40, Tween-80, Brij-35), diglycerol laurate, diglycerol caprate, diglycerol caprylate, diglycerol monocaprylate, polyglycerol laurate, polyglycerol caprate and polyglycerol caprylate.

The term “non-aqueous” as used herein refers to a composition to which no water is added during preparation of the pharmaceutical composition. It is known to the person skilled in the art that a composition which has been prepared without addition of water may take up small amounts of water from the surroundings during handling of the pharmaceutical composition such as e.g. a soft-capsule or a hard-capsule used to encapsulate the composition. Also, the insulin peptide and/or one or more of the excipients in the pharmaceutical composition may have small amounts of water bound to it before preparing a pharmaceutical composition according to the invention. A non-aqueous pharmaceutical composition according to the invention may thus contain small amounts of water. In one aspect a non-aqueous pharmaceutical composition according to the invention comprises less than 10%(w/w) water. In another aspect, the composition according to the invention comprises less than 5%(w/w) water. In another aspect, the composition according to the invention comprises less than 4%(w/w) water, in another aspect less than 3%(w/w) water, in another aspect less than 2%(w/w) water and in yet another aspect less than 1%(w/w) water. In one aspect the composition accord 0%(w/w) water

Examples of other non-ionic surfactants include, but are not limited to: diglycerol monocaprylate, Tween 20, Tween 40, Tween 60, Tween 80, Span 40, poloxamer 124

When used herein the term “Hydrophilic-lipophilic balance” or “HLB” of a surfactant or lipophilic component is a measure of the degree to which it is hydrophilic or lipophilic, determined by calculating values for the different regions of the molecule, as described by Griffin (Griffin W C: “Classification of Surface-Active Agents by ‘HLB,’” Journal of the Society of Cosmetic Chemists 1 (1949): 311) or by Davies (Davies J T: “A quantitative kinetic theory of emulsion type, I. Physical chemistry of the emulsifying agent,” Gas/Liquid and Liquid/Liquid Interface. Proceedings of the International Congress of Surface Activity (1957): 426-438).

“Non-ionic surfactants with HLB above 10” are a selection of non-ionic surfactants which have the common feature of having HLB above 10.

For exemplification, a non-limiting list of surfactants with HLB above 10 is provided below together with their HLB value:

Polyethylene glycol sorbitane monolaurate (Tween 20, Polysorbate 20, super refined polysorbate 20) with an HLB of 16.7;
Polyoxyethylene (20) sorbitan monooleate (Tween 80, Polysorbate 80, super refined polysorbate 80) with an HLB of 15;
Polyoxyethylene (20) sorbitan monopalmitate (Tween 40, Polysorbate 40, super refined polysorbate 40) with an HLB of 15.6;
Diglycerol caprylate (diglycerol monocaprylate, polyglycerol caprylate) with an HLB of 11. Polyglycerol caprate (Rylo PG10 Pharma) with HLB of 10;
Caprylocaproyl macrogolglycerides (Labrasol, Labrasol ALF) with an HLB of 14;
Block polymers such as SYNPERONIC PE/L 44 (Poloxamer 124);
Polyoxyethylenestearate (Myrj 45, Macrogolstearate) with HLB of 11.1;
Polyoxyethylenestearate (Myrj 49, Macrogolstearate) with HLB of 15;
Polyoxyethylenestearate (Myrj 51, Macrogolstearate) with HLB of 16;
Polyoxyethylenestearate (Myrj 52, Macrogolstearate) with HLB of 16.9;
Polyoxyethylenestearate (Myrj 53, Macrogolstearate) with HLB of 17.9;
Polyoxyethylenestearate (Myrj 59, Macrogolstearate) with HLB of 18.8; and
Polyoxyethyleneglyceroltriricinoleat (Cremophor EL) with HLB of 13.3.

As used herein the term “amino acid” refers to any molecule that contains both amine and carboxyl functional groups.

The term “enteric coating” as used herein means a polymer coating that controls disintegration and release of the solid oral dosage form. The site of disintegration and release of the solid dosage form may be designed depending on the pH of the targeted area, where absorbtion of the therapeutic peptide or protein (i.e. therapeutical active peptide or protein) is desired, thus does also include acid resistant protective coatings. The term includes known enteric coatings, but also any other coating with enteric properties, wherein said term “enteric properties” means properties controlling the disintegration and release of the solid oral dosage form (i.e. the oral pharmaceutical composition according to this invention).

The term “enteric soft- or hard capsule technology” when used herein means soft- or hard capsule technology comprising at least one element with enteric properties, such as at least one layer of an enteric coating. The term “delayed release coatings” as used herein means a polymer coating which releases the API in a delayed manner after oral dosing. Delayed release can be achieved by pH dependent or pH independent polymer coatings.

The term “co-surfactant” when used herein refers to an additional surfactant added to a composition or formulation, wherein a first surfactant is present.

In the present context, 1,2-propanediol and propylene glycol is used interchangeably.

The following method can be used to measure the in vivo effect of the FA-Daas according to the present inventions or compound comprising FA-Daas according to the present invention.

An insulin derivative (60 nmol/kg) is dissolved in phosphate buffer (pH 7.4) in presence of fatty acid acylated amino acids. The composition is injected into mid-jejunum of anaesthetized overnight fasted Sprague-Dawley rats (n=6) and the pharmacokinetic profile is obtained by determining concentration of this insulin derivative (using ELISA, LOCI or LC-MS protocols) in plasma samples taken at different time points.

The term “diabetes” or “diabetes mellitus” includes type 1 diabetes, type 2 diabetes, gestational diabetes (during pregnancy) and other states that cause hyperglycaemia. The term is used for a metabolic disorder in which the pancreas produces insufficient amounts of insulin, or in which the cells of the body fail to respond appropriately to insulin thus preventing cells from absorbing glucose. As a result, glucose builds up in the blood.

Type 1 diabetes, also called insulin-dependent diabetes mellitus (IDDM) and juvenile-onset diabetes, is caused by B-cell destruction, usually leading to absolute insulin deficiency.

Type 2 diabetes, also known as non-insulin-dependent diabetes mellitus (NIDDM) and adult-onset diabetes, is associated with predominant insulin resistance and thus relative insulin deficiency and/or a predominantly insulin secretory defect with insulin resistance.

In one aspect, pharmaceutical compostion according to the present invention according to the invention is used for the preparation of a medicament for the treatment or prevention of hyperglycemia including stress induced hyperglycemia, type 2 diabetes, impaired glucose tolerance, type 1 diabetes, burns, operation wounds, other diseases or injuries where an anabolic effect is needed in the treatment, myocardial infarction, stroke, coronary heart disease, other cardiovascular disorders, treatment of critically ill diabetic and non-diabetic patients and polyneuropathy.

In another aspect, [an insulin/insulin analogue/insulin derivative] according to the invention is used as a medicament for delaying or preventing disease progression in type 2 diabetes.

In one aspect of the invention, the pharmaceutical compostion according to the present invention is for use as a medicament for the treatment or prevention of hyperglycemia including stress induced hyperglycemia, type 2 diabetes, impaired glucose tolerance, type 1 diabetes, and burns, operation wounds and other diseases or injuries where an anabolic effect is needed in the treatment, myocardial infarction, stroke, coronary heart disease and other cardiovascular disorders.

In a further aspect the invention is related to a method for the treatment or prevention of hyperglycemia including stress induced hyperglycemia, type 2 diabetes, impaired glucose tolerance, type 1 diabetes, and burns, operation wounds and other diseases or injuries where an anabolic effect is needed in the treatment, myocardial infarction, coronary heart disease and other cardiovascular disorders, stroke, the method comprising administering to a patient in need of such treatment an effective amount for such treatment of pharmaceutical compostion according to the present invention according to the invention.

The term “treatment” is meant to include both the prevention and minimization of the referenced disease, disorder, or condition (i.e., “treatment” refers to both prophylactic and therapeutic administration of a pharmaceutical compostion according to the present invention unless otherwise indicated or clearly contradicted by context.

The route of administration may be any route which effectively transports a compound of this invention to the desired or appropriate place in the body, such as parenterally, for example, subcutaneously, intramuscularly or intraveneously. Alternatively, a compound of this invention can be administered orally, pulmonary, rectally, transdermally, buccally, sublingually, or nasally.

For parenterally administration, a compound of this invention is formulated analogously with the formulation of known insulins. Furthermore, for parenterally administration, a compound of this invention is administered analogously with the administration of known insulins and the physicians are familiar with this procedure.

The amount of a compound of this invention to be administered, the determination of how frequently to administer a compound of this invention, and the election of which compound or compounds of this invention to administer, optionally together with another antidiabetic compound, is decided in consultation with a practitioner who is familiar with the treatment of diabetes.

The Following is a Non-Limiting List of Embodiments Further Comprised within the Scope of the Invention:
1. A pharmaceutical composition comprising
   a. At least one FA-Daa or a salt thereof represented by the general formula A-Xy, wherein A is a non-polar uncharged or acidic amino acid and Xy is a fatty acid moiety attached by acylation to A's alpha amino group and y represents the number of carbon atoms in said fatty acid moiety, wherein y is 10, 12, 14, 16 or 18 when said amino acid is a non-polar uncharged amino acid and y is 16 or 18 when said amino acid is an acidic, wherein the stereo configuration of the chiral carbon atom in the amino acid moiety is D and
   b. a hydrophilic peptide or protein.
2. The pharmaceutical composition according to embodiment 1, wherein said composition is an oral composition.
3. The pharmaceutical composition according to any one of the embodiments 1 or 2, wherein said non-polar uncharged amino acid is selected from the group consisting of Alanine, Isoleucine, Leucine, Proline and Valine and the acidic amino acid is selected from the group consisting of Aspartic acid and Glutamic acid.
4. The pharmaceutical composition according to any one of the embodiments 1 or 3, wherein said non-polar uncharged amino acid is selected from the group consisting of Alanine, Isoleucine, Leucine, Proline and Valine.
5. The pharmaceutical composition according to any one of the embodiments 1 or 3, wherein the acidic amino acid is selected from the group consisting of Aspartic acid and Glutamic acid
6. The pharmaceutical composition according to any one of the embodiments 1-5, wherein y is 18.
7. The pharmaceutical composition according to any one of the embodiments 1-5, wherein y is 16.
8. The pharmaceutical composition according to any one of the embodiments 1-4, wherein y is 14.
9. The pharmaceutical composition according to any one of the embodiments 1-4, wherein y is 12.
9A. The pharmaceutical composition according to any one of the embodiments 1-4, wherein y is 10.
10. The oral pharmaceutical composition according to any one of the embodiments 1 to 12 comprising
   a. one or more FA-Daa or salt thereof represented by the general formula;

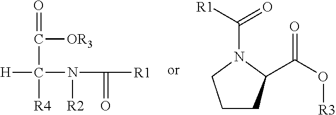

wherein R1 is a hydrocarbon chain comprising 11 to 17 carbons, R2 is either H (i.e. hydrogen) or CH₃ (i.e. methyl group), R3 is either H, or absent, and R4 is an amino acid side chain, wherein the stereo configuration of the chiral carbon atom in the amino acid moiety is D, with the proviso that when R4 is from a non-polar uncharged amino acid, then R1 comprises 11, 13, 15 or 17 carbon atoms and when R4 is from an acidic amino acid, then R1 comprises 15 or 17 carbon atoms and
   b. a hydrophilic peptide or protein.
11. The oral pharmaceutical composition according to any one of the preceding claims selected from the group consisting of general formula (h), (i), (j), (l), (m) and (n).
12. The oral pharmaceutical composition according to any one of the embodiments 1 to 10 comprising
   a. one or more FA-Daa or salt thereof represented by the general formula;

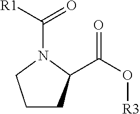

wherein R1 is a hydrocarbon chain comprising 11 to 17 carbons and R3 is either H, or absent and
   b. a hydrophilic peptide or protein.
13. The oral pharmaceutical composition according to any one of the preceding embodiments is of the general formula (k).
14. The oral pharmaceutical composition according to any one of the embodiments 1 to 9 comprising
   a. one or more FA-Daa or salt thereof represented by the general formula;

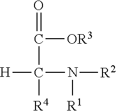

wherein R1 is a fatty acid chain comprising 12 to 18 carbons, R2 is either H (i.e. hydrogen) or CH₃ (i.e. methyl group), R3 is either H, or absent, and R4 is an amino acid side chain, wherein the stereo configuration of the chiral carbon atom in the amino acid moiety is D, with the proviso that when R4 is from a non-polar uncharged amino acid, then R1 comprises 12, 14, 16 or 18 carbon atoms and when R4 is from an acidic amino acid, then R1 comprises 16 or 18 carbon atoms.
   a. a hydrophilic peptide or protein.
15. The oral pharmaceutical composition according to any one of the embodiments 1 to 11, wherein R1 comprises 13 to 17 carbon atoms, with the proviso that when R4 is from a non-polar uncharged amino acid, then R1 comprises 13, 15 or 17 carbon atoms and when R4 is from an acidic amino acid, then R1 comprises 15 or 17 carbon atoms.
16. The oral pharmaceutical composition according to any one of the embodiments 1 to 13, wherein R1 comprises 15 to 17 carbon atoms.
17. The oral pharmaceutical composition according to any one of the embodiments 1 to 13, wherein R1 comprises 15 carbon atoms.
18. The oral pharmaceutical composition according to any one of the embodiments 1 to 13, wherein R1 comprises 17 carbon atoms.
19. The oral pharmaceutical composition according to any one of the embodiments 1 to 13, wherein R1 comprises 11 to 13 carbon atoms, with the proviso that R4 is from a non-polar uncharged amino acid.
20. The oral pharmaceutical composition according to any one of the embodiments 1 to 13, wherein R1 comprises 13 carbon atoms, with the proviso that R4 is from a non-polar uncharged amino acid.
21. The oral pharmaceutical composition according to any one of the embodiments 1 to 13, wherein R1 comprises 11 carbon atoms, with the proviso that R4 is from a non-polar uncharged amino acid.
22. The oral pharmaceutical composition according to any one of the embodiments 1-11 and 14, wherein R1 comprises 12 carbon atoms.
23. The oral pharmaceutical composition according to any one of the embodiments 1-11 and 14, wherein R1 comprises 14 carbon atoms.
24. The oral pharmaceutical composition according to any one of the embodiments 1-11 and 14, wherein R1 comprises 16 carbon atoms.
25. The oral pharmaceutical composition according to any one of the embodiments 1-11 and 14, wherein R1 comprises 18 carbon atoms.
26. The oral pharmaceutical composition according to any one of the preceding embodiments wherein said salt is selected from the group consisting of sodium (Na+) and potassium (K+).
27. The oral pharmaceutical composition according to any one of the preceding embodiments wherein said salt is selected from the group consisting of sodium (Na+) and potassium (K+).
28. The oral pharmaceutical composition according to any one of the preceding embodiments wherein said salt is a sodium (Na+) salt.
29. The oral pharmaceutical composition according to any one of the preceding embodiments wherein said salt is a potassium (K+).
30. The oral composition according to any one of the preceeding embodiments wherein said hydrophilic peptide or protein is an insulin.
31. The oral composition according to any one of the preceeding embodiments wherein said hydrophilic peptide or protein is an insulin peptide or protein.
32. The oral composition according to any one of the preceeding embodiments wherein said hydrophilic peptide or protein is an insulin analogue or derivative.
33. The oral composition according to any one of the preceeding embodiments further comprising an enteric or delayed release coating.
34. The oral pharmaceutical composition according to any one of the embodiments 1-33, wherein the amino acid residue of said FA-Daa is selected from the group consisting of: Sodium or potassium lauroyl D-alaninate, N-dodecanoyl-D-alanine, Sodium or potassium myristoyl D-Alaninate, N-tetradecanoyl D-Alanine, Sodium or potassium palmitoyl D-Alaninate, N-hexadecanoyl D-Alanine, Sodium or potassium stearoyl D-Alaninate, N-octadecanoyl D-Alanine, Sodium or potassium lauroyl D-Isoleucinate, N-dodecanoyl-D-Isoleucine, Sodium or potassium myristoyl D-Isoleucinate, N-tetradecanoyl D-Isoleucine, Sodium or potassium palmitoyl D-Isoleucinate, N-hexadecanoyl D-Isoleucine, Sodium or potassium stearoyl D-Isoleucinate, N-octadecanoyl D-Isoleucine, Sodium or potassium lauroyl D-Leucinate, N-dodecanoyl-D-Leucine, Sodium or potassium myristoyl D-Leucinate, N-tetradecanoyl D-Leucine, Sodium or potassium palmitoyl D-Leucinate, N-hexadecanoyl D-Leucine, Sodium or potassium stearoyl D-Leucinate, N-octadecanoyl D-Leucine, Sodium or potassium lauroyl D-Prolinate, N-dodecanoyl-D-Proline, Sodium or potassium myristoyl D-Prolinate, N-tetradecanoyl D-Proline, Sodium or potassium palmitoyl D-Prolinate, N-hexadecanoyl D-Proline, Sodium or potassium stearoyl D-Prolinate, N-octadecanoyl D-Proline, Sodium or potassium lauroyl D-Valinate, N-dodecanoyl-D-Valine, Sodium or potassium myristoyl D-Valinate, N-tetradecanoyl D-Valine, Sodium or potassium palmitoyl D-Valinate, N-hexadecanoyl D-Valine, Sodium or potassium stearoyl D-Valinate, N-octadecanoyl D-Valine Sodium or potassium palmitoyl D-Aspartate, N-hexadecanoyl D-Aspartic acid, Sodium or potassium palmitoyl D-Glutamate, N-hexadecanoyl D-Glutamic acid, Sodium or potassium stearoyl D-Aspartate, N-octadecanoyl D-Aspartic acid, Sodium or potassium stearoyl D-Glutamate and N-octadecanoyl D-Glutamic acid.
35. The oral pharmaceutical composition according to any one of the embodiments 1-3, 5-10 and 14-33, wherein the amino acid residue of said FA-Daa is selected from the group consisting of: Sodium or potassium palmitoyl D-Aspartate, N-hexadecanoyl D-Aspartic acid, Sodium or potassium palmitoyl D-Glutamate, N-hexadecanoyl D-Glutamic acid, Sodium or potassium stearoyl D-Aspartate, N-octadecanoyl D-Aspartic acid, Sodium or potassium stearoyl D-Glutamate and N-octadecanoyl D-Glutamic acid.
36. The oral pharmaceutical composition according to any one of the embodiments 1-4 and 6-33, wherein the amino acid residue of said FA-Daa is selected from the group consisting of: Sodium or potassium lauroyl D-alaninate, N-dodecanoyl-D-alanine, Sodium or potassium lauroyl D-Isoleucinate, N-dodecanoyl D-Isoleucine, Sodium or potassium lauroyl D-Leucinate, N-dodecanoyl-D-Leucine, Sodium or potassium lauroyl D-Prolinate, N-dodecanoyl-D-Proline, Sodium or potassium lauroyl D-Valinate, N-dodecanoyl-D-Valine, Sodium or potassium lauroyl D-alaninate, N-dodecanoyl-D-alanine, Sodium or potassium lauroyl D-Isoleucinate, N-dodecanoyl-D-Isoleucine, Sodium or potassium lauroyl D-Leucinate, N-dodecanoyl-D-Leucine, Sodium or potassium lauroyl D-Prolinate, N-dodecanoyl-D-Proline, Sodium or potassium lauroyl D-Valinate and N-dodecanoyl-D-Valine.
37. The oral pharmaceutical composition according to any one of the embodiments 1-4 and 6-33, wherein the amino acid residue of said FA-Daa is selected from the group consisting of: Sodium or potassium lauroyl D-alaninate, N-dodecanoyl-D-alanine, Sodium or potassium myristoyl D-Alaninate, N-tetradecanoyl D-Alanine, Sodium or potassium palmitoyl D-Alaninate, N-hexadecanoyl D-Alanine, Sodium or potassium stearoyl D-Alaninate and N-octadecanoyl D-Alanine.
38. The oral pharmaceutical composition according to any one of the embodiments 1-4 and 6-33, wherein the amino acid residue of said FA-Daa is selected from the group consisting of: Sodium or potassium lauroyl D-alaninate, N-dodecanoyl-D-alanine, Sodium or potassium myristoyl D-Alaninate, N-tetradecanoyl D-Alanine, Sodium or potassium palmitoyl D-Alaninate, N-hexadecanoyl D-Alanine, Sodium or potassium stearoyl D-Alaninate and N-octadecanoyl D-Alanine.
39. An oral pharmaceutical composition according to any one of the 1-4 and 6-33, wherein the amino acid residue of said FA-Daa is selected from the group consisting of: Sodium or potassium lauroyl D-alaninate, N-dodecanoyl-D-alanine, Sodium or potassium myristoyl D-Alaninate, N-tetradecanoyl D-Alanine, Sodium or potassium palmitoyl D-Alaninate, N-hexadecanoyl D-Alanine, Sodium or potassium stearoyl D-Alaninate and N-octadecanoyl D-Alanine.
40. The oral pharmaceutical composition according to any one of the embodiments 1-4 and 6-33 wherein the amino acid residue of said FA-Daa is selected from the group consisting of: Sodium or potassium lauroyl D-Isoleucinate, N-dodecanoyl-D-Isoleucine, Sodium or potassium myristoyl D-Isoleucinate, N-tetradecanoyl D-Isoleucine, Sodium or potassium palmitoyl D-Isoleucinate, N-hexadecanoyl D-Isoleucine, Sodium or potassium stearoyl D-Isoleucinate and N-octadecanoyl D-Isoleucine.
41. The oral pharmaceutical composition according to any one of the embodiments 1-4 and 6-33, wherein the amino acid residue of said FA-Daa is selected from the group consisting of: Sodium or potassium lauroyl D-Leucinate, N-dodecanoyl-D-Leucine, Sodium or potassium myristoyl D-Leucinate, N-tetradecanoyl D-Leucine, Sodium or potassium palmitoyl D-Leucinate, N-hexadecanoyl D-Leucine, Sodium or potassium stearoyl D-Leucinate and N-octadecanoyl D-Leucine.
42. The oral pharmaceutical composition according to any one of the embodiments 1-4 and 6-33, wherein the amino acid residue of said FA-Daa is selected from the group consisting of: Sodium or potassium lauroyl D-Prolinate, N-dodecanoyl-D-Proline, Sodium or potassium myristoyl D-Prolinate, N-tetradecanoyl D-Proline, Sodium or potassium palmitoyl D-Prolinate, N-hexadecanoyl D-Proline, Sodium or potassium stearoyl D-Prolinate and N-octadecanoyl D-Proline.
43. The oral pharmaceutical composition according to any one of the embodiments 1-4 and 6-33, wherein the amino acid residue of said FA-Daa is selected from the group consisting of: Sodium or potassium lauroyl D-Valinate, N-dodecanoyl-D-Valine, Sodium or potassium myristoyl D-Valinate, N-tetradecanoyl D-Valine, Sodium or potassium palmitoyl D-Valinate, N-hexadecanoyl D-Valine, Sodium or potassium stearoyl D-Valinate and N-octadecanoyl D-Valine.
44. The oral pharmaceutical composition according to any one of the embodiments 1-4 and 6-33, wherein the amino acid residue of said FA-Daa is selected from the group consisting of: Sodium or potassium lauroyl D-alaninate, N-dodecanoyl-D-alanine, Sodium or potassium lauroyl D-Isoleucinate, N-dodecanoyl D-Isoleucine, Sodium or potassium lauroyl D-Leucinate, N-dodecanoyl-D-Leucine, Sodium or potassium lauroyl D-Prolinate, N-dodecanoyl-D-Proline, Sodium or potassium lauroyl D-Valinate and N-dodecanoyl-D-Valine.
45. The oral pharmaceutical composition according to any one of the embodiments 1-4 and 6-33, wherein the amino acid residue of said FA-Daa is selected from the group consisting of: Sodium or potassium lauroyl D-alaninate, N-dodecanoyl-D-alanine, Sodium or potassium lauroyl D-Isoleucinate, N-dodecanoyl-D-Isoleucine, Sodium or potassium lauroyl D-Leucinate, N-dodecanoyl-D-Leucine, Sodium or potassium lauroyl D-Prolinate, N-dodecanoyl-D-Proline, Sodium or potassium lauroyl D-Valinate and N-dodecanoyl-D-Valine.
46. The oral pharmaceutical composition according to any one of the embodiments 1-4 and 6-33, wherein the amino acid residue of said FA-Daa is selected from the group consisting of: Sodium or potassium myristoyl D-Alaninate, N-tetradecanoyl D-Alanine, Sodium or potassium myristoyl D-Isoleucinate, N-tetradecanoyl D-Isoleucine, Sodium or potassium myristoyl D-Leucinate, N-tetradecanoyl D-Leucine, Sodium or potassium myristoyl D-Prolinate, N-tetradecanoyl D-Proline, Sodium or potassium myristoyl D-Valinate and N-tetradecanoyl D-Valine.
47. The oral pharmaceutical composition according to any one of the embodiments 1-4 and 6-33, wherein the amino acid residue of said FA-Daa is selected from the group consisting of: Sodium or potassium palmitoyl D-Alaninate, N-hexadecanoyl D-Alanine, Sodium or potassium palmitoyl D-Isoleucinate, N-hexadecanoyl D-Isoleucine, Sodium or potassium palmitoyl D-Leucinate, N-hexadecanoyl D-Leucine, Sodium or potassium palmitoyl D-Prolinate, N-hexadecanoyl D-Proline, Sodium or potassium palmitoyl D-Valinate and N-hexadecanoyl D-Valine.
48. The oral pharmaceutical composition according to any one of the embodiments 1-4 and 6-33, wherein the amino acid residue of said FA-Daa is selected from the group consisting of: Sodium or potassium stearoyl D-Alaninate, N-octadecanoyl D-Alanine, Sodium or potassium stearoyl D-Isoleucinate, N-octadecanoyl D-Isoleucine, Sodium or potassium stearoyl D-Leucinate, N-octadecanoyl D-Leucine, Sodium or potassium stearoyl D-Prolinate, N-octadecanoyl D-Proline, Sodium or potassium stearoyl D-Valinate and N-octadecanoyl D-Valine.
49. The oral pharmaceutical composition according to any one of the embodiments 1-3, 5-10 and 14-33, wherein the amino acid residue of said FA-Daa is selected from the group consisting of: Sodium or potassium palmitoyl D-Aspartate, N-hexadecanoyl D-Aspartic acid, Sodium or potassium palmitoyl D-Glutamate and N-hexadecanoyl D-Glutamic acid.
50. The oral pharmaceutical composition according to any one of the embodiments 1-3, 5-10 and 14-33, wherein the amino acid residue of said FA-Daa is selected from the group consisting of: Sodium or potassium stearoyl D-Aspartate, N-octadecanoyl D-Aspartic acid, Sodium or potassium stearoyl D-Glutamate and N-octadecanoyl D-Glutamic acid.
51. The oral pharmaceutical composition according to any one of the preceeding embodiments, wherein the amino acid residue of said FA-Daa is selected from the group consisting of the combinations possible from Table 1.
51A. The oral pharmaceutical composition according to any one of the preceeding embodiments, wherein the amino acid residue of said FA-Daa is selected from the group consisting of the combinations possible from Table 1A.
52. The oral pharmaceutical composition according to any one of the preceeding embodiments, wherein the amino acid residue of said FA-Daa is selected from the group consisting of the combinations possible from Table 2.
53. The oral pharmaceutical composition according to any one of the preceeding embodiments, further comprising other pharmaceutical excipients.
54. The oral pharmaceutical composition according to any one of the preceeding embodiments for use as a medicament.
55. The oral pharmaceutical composition according to any one of the preceeding embodiments for use as a medicament for treatment of Diabetes Mellitus.
56. The pharmaceutical composition according to any one of the preceding embodiments, wherein said hydrophilic peptide or protein is an insulin peptide.
57. The oral composition according to any one of the preceeding embodiments further comprising an enteric or delayed release coating.
58. The oral pharmaceutical composition according to any one of the preceeding embodiments, wherein the fatty acid acylated amino acid is in the form of its free acid or salt.
59. The oral pharmaceutical composition according to any one of the preceeding embodiments, further comprising propylene glycol.
60. The oral pharmaceutical composition according to any one of the preceeding embodiments, further comprising SEDDS, SMEDDS or SNEDDS.
61. The pharmaceutical composition according to any one of the preceding embodiments, which comprises less than 10% (w/w) water.
62. The oral pharmaceutical composition according to any one of the preceeding embodiments, further comprising other pharmaceutical excipients.
63. The oral pharmaceutical composition according to any one of the preceeding embodiments for use as a medicament.
64. The oral pharmaceutical composition according to any one of the preceeding embodiments for use as a medicament for treatment of Diabetes Mellitus.
65. Use of an oral pharmaceutical composition according to any one of the preceeding embodiments, for increasing the bioavailability of said hydrophilic peptide or protein.
66. Use of an oral pharmaceutical composition according to any one of the preceeding embodiments, for increasing the bioavailability of said therapeutic macromolecule.
67. Use of an oral pharmaceutical composition according to any one of the preceeding embodiments, for increasing the bioavailability of said therapeutic active peptide.
68. A method for increasing bioavailability of an insulin peptide or protein comprising the steps of including a FA-aa in a pharmaceutical composition of insulin peptides or proteins administered to an individual.
69. A method for increasing the plasma concentration of insulin, insulin peptide or protein or insulin analouges or derivatives comprising the step of exposing the gastrointestinal tract of an individual to a pharmaceutical composition comprising an of insulin, insulin peptide or protein or insulin analouges or derivatives and a FA-aa resulting in an increased plasma concentration of said insulin peptide or protein in said individual.
70. The method of embodiment 62, wherein said exposure is achieved by oral administration of said pharmaceutical composition.
71. A method for increasing the up-take of an of insulin, insulin peptide or protein or insulin analouges or derivatives comprising the step of: exposing the gastrointestinal tract of an individual to a of insulin, insulin peptide or protein or insulin analouges or derivatives and at least one FA-aa, whereby the plasma concentration of said insulin, insulin peptide or protein or insulin analouges or derivatives in said individual is increased compared to an exposure not including the at least one FA-aa.
72. A method for treatment of insulin related disorders or diseases comprising administering a pharmaceutical composition comprising an insulin peptide or protein and at least one FA-aa.
73. A method for treatment of Diabetes Mellitus comprising administering a pharmaceutical composition comprising an insulin peptide or protein compound and at least one FA-aa.
74. A method for increasing uptake of an insulin peptide or protein across the mucous membrane of the gastro intestinal tract comprising the steps of, administering a pharmaceutical composition comprising an insulin peptide or protein and at least one FA-aa to an individual, whereby an increased uptake of said insulin peptide or protein is obtained compared to the uptake of said insulin peptide or protein obtained when said growth hormone composition does not including the at least one FA-aa.
75. A method for increasing uptake of an insulin peptide or protein across the epithelia cell layer of the gastro intestinal tract comprising the steps of, administering a pharmaceutical composition comprising an insulin peptide or protein and at least one FA-aa to an individual, whereby an increased uptake of said insulin peptide or protein is obtained compared to the uptake of said insulin peptide or protein obtained when said growth hormone composition does not including the at least one FA-aa.
76. A method for increasing uptake of an insulin peptide or protein across the mucous membrane of the gastro intestinal tract comprising the steps of, administering a pharmaceutical composition comprising an insulin peptide or protein and at least one FA-aa to an individual, whereby an increased uptake of said insulin peptide or protein is obtained compared to the uptake of said insulin peptide or protein obtained when said growth hormone composition does not including the at least one FA-aa.
77. The method of embodiment 68-76, wherein the pharmaceutical composition is described by any one of embodiments 1-67.
78. A method for the manufacture of compositions according to the present invention comprising the step of dissolving insulin in propylene glycol.
79. A method for the manufacture of compositions according to the present invention comprising the step of mixing said FA-Daa to a mixture of an insulin peptide or protein and the ingredients for SEDDS, SMEDDS or SNEDDS.

### EXAMPLES

### Example 1

Liquid formulations comprising insulin, SEDDS, SMEDDS or SNEDDS formulations were prepared according to the guidance given in WO08145728 example 1 and 2, pages 53-54 wherein the FA-Daa according to this invention were added to the isulin solution. Insulin was dissolved in the solvent (propylene glycol, water and/or glycerol), The FA-Daa was then dissolven in said insulin solution whereupon the lipid phase components of SEDDS, SMEDDS or SNEDDS were added to this mixture followed by the surfactants. All formulations contained insulin derivative A14E, B25H, B29K(N(eps)Octadecanedioyl-gGlu-OEG-OEG), desB30 human insulin (5 mg/g). The insulin was first dissolved in water and the pH adjusted to pH 7.4 with a non volatile base (NaOH) followed by freeze drying, the resulting insulin powder was then dissolved first in propylene glycol and then mixed with FA-Daa and other excipients. Liquid formulations comprising insulin, FA-Daa's according to the present invention and SEDDS, SMEDDS or SNEDDS formulations were prepared comprising different FA-Daa salts, propylene glycol, polysorbate 20 and diglycerol monocaprylate. Mean particle size (hydrodynamic diameter) was analysed after 10 fold dilution in MilliQ water at 37° C. and respective PDI (polydispersity index). The compositions and the results of particle size analysis are shown in table 3.

TABLE 3
Insulin SEDDS and SMEDDS compositions comprising different fatty acid acylated D-
aminoacid salts, propylene glycol, polysorbate 20 and diglycerol monocaprylate.

| | | | fatty acid | | | Average | |
|---|---|---|---|---|---|---|---|
| | | Pro- | acylated D- | Poly- | Diglycerol | paricle size | Poly- |
| | Insulin | pylene | amino acid, | sorbate | mono- | diameter | dispersity |
| No | analogue | glycol | 3% | 20 | caprylate | (nm) | index |
| 1 | 5 mg/ml | 15% | C10-D- | 30% | 51.5% | 5.233 | 0.268 |
| | | | Leucinate | | | | |
| | | | potassium | | | | |
| 2 | 5 mg/ml | 15% | C12-D- | 30% | 51.5% | 5.016 | 0.261 |
| | | | Leucinate | | | | |
| | | | potassium | | | | |
| 3 | 5 mg/ml | 15% | C14-D- | 30% | 51.5% | 12.65 | 0.209 |
| | | | Glutamate | | | | |
| | | | potassium | | | | |
| 4 | 5 mg/ml | 15% | C16-D- | 30% | 51.5% | 10.52 | 0.455 |
| | | | Glutamate | | | | |
| | | | potassium | | | | |
| 5 | 5 mg/ml | 15% | C16-D- | 30% | 51.5% | 6.213 | 0.327 |
| | | | Aspartate | | | | |
| | | | potassium | | | | |
| 6 | 5 mg/ml | 15% | C12-D- | 30% | 51.5% | 13.63 | 0.308 |
| | | | Alanine | | | | |
| | | | potassium | | | | |
| 7 | 5 mg/ml | 15% | C12-D- | 30% | 51.5% | 7.461 | 0.343 |
| | | | Phenylalanine | | | | |
| | | | potassium | | | | |
| 8 | 5 mg/ml | 15% | C14-D- | 30% | 51.5% | 5.31 | 0.268 |
| | | | Aspartate | | | | |
| | | | potassium | | | | |
| 9 | 5 mg/ml | 15% | C14-D- | 30% | 51.5% | 6.263 | 0.309 |
| | | | Tryptophane | | | | |
| | | | potassium | | | | |
| 10 | 5 mg/ml | 15% | C14-D- | 30% | 51.5% | 78.3 | 0.916 |
| | | | Tyrosine | | | | |
| | | | potassium | | | | |
| 11 | 5 mg/ml | 15% | C12-D- | 30% | 51.5% | 289.9 | 0.57 |
| | | | Isoleucinate | | | | |
| | | | potassium | | | | |
| 12 | 5 mg/ml | 15% | C12-D- | 30% | 51.5% | 96.98 | 0.223 |
| | | | Proline | | | | |
| | | | potassium | | | | |
| 13 | 5 mg/ml | 15% | C12-D- | 30% | 51.5% | 5.047 | 0.266 |
| | | | Valine | | | | |
| | | | potassium | | | | |

### Example 2

Liquid formulations comprising insulin, SEDDS, SMEDDS or SNEDDS formulations were prepared according to the guidance given in WO08145728 example 1 and 2, pages 53-54 wherein the FA-Daa according to this invention were added to the isulin solution. Insulin was dissolved in the solvent (propylene glycol, water and/or glycerol), The FA-Daa was then dissolven in said insulin solution whereupon the lipid phase components of SEDDS, SMEDDS or SNEDDS were added to this mixture followed by the surfactants. All formulations contained insulin derivative A14E, B25H, B29K(N(eps)Octadecanedioyl-gGlu-OEG-OEG), desB30 human insulin (5 mg/g). The insulin was first dissolved in water and the pH adjusted to pH 7.4 with a non volatile base (NaOH) followed by freeze drying, the resulting insulin powder was then dissolved first in propylene glycol and then mixed with the other excipients. Liquid formulations comprising insulin, FA-Daa's according to the present invention and SEDDS, SMEDDS or SNEDDS formulations were prepared comprising different amounts of C12-D-leucinate potassium, propylene glycol, polysorbate 20 and diglycerol monocaprylate. Mean particle size (hydrodynamic diameter) was analysed after 10 fold dilution in MilliQ water at 37° C. and respective PDI (polydispersity index). The compositions and the results of particle size analysis are shown in table 4.

TABLE 4
Insulin SEDDS and SMEDDS compositions comprising different amounts of C12-D-leucinate
potassium, propylene glycol, polysorbate 20 and diglycerol monocaprylate.

| | | | C12-D- | | | Average | |
|---|---|---|---|---|---|---|---|
| | | Pro- | Leucinate | Poly- | Diglycerol | paricle size | Poly- |
| | Insulin | pylene | potas- | sorbate | mono- | diameter | dispersity |
| No | analogue | glycol | sium | 20 | caprylate | (nm) | index |
| 1 | 5 mg/ml | 15% | 3% | 30% | 51.5% | 4.866 | 0.269 |
| 2 | 5 mg/ml | 15% | 5% | 30% | 49.5% | 4.061 | 0.255 |
| 3 | 5 mg/ml | 15% | 7% | 30% | 47.5% | 10.81 | 0.335 |
| 4 | 5 mg/ml | 15% | 10% | 30% | 44.5% | 6.303 | 0.171 |

### Example 3

Liquid formulations comprising insulin, SEDDS, SMEDDS or SNEDDS formulations were prepared according to the guidance given in WO08145728 example 1 and 2, pages 53-54 wherein the FA-Daa according to this invention were added to the isulin solution. Insulin was dissolved in the solvent (propylene glycol, water and/or glycerol), The FA-Daa was then dissolven in said insulin solution whereupon the lipid phase components of SEDDS, SMEDDS or SNEDDS were added to this mixture followed by the surfactants. All formulations contained insulin derivative A14E, B25H, B29K(N(eps)Octadecanedioyl-gGlu-OEG-OEG), desB30 human insulin (5 mg/g). The insulin was first dissolved in water and the pH adjusted to pH 7.4 with a non volatile base (NaOH) followed by freeze drying, the resulting insulin powder was then dissolved first in propylene glycol, water or glycerol and then mixed with the other excipients. Liquid formulations comprising insulin, FA-Daa's according to the present invention and SEDDS, SMEDDS or SNEDDS formulations were prepared comprising C10-D-leucinate potassium, polysorbate 20 and different solvents and lipids/co-surfactants. Mean particle size (hydrodynamic diameter) was analysed after 10 fold dilution in MilliQ water at 37° C. and respective PDI (polydispersity index). The compositions and the results of particle size analysis are shown in table 5.

TABLE 5
Insulin SEDDS and SMEDDS compositions comprising C10-D-leucinate
potassium, polysorbate 20 and different solvents and lipids/co-surfactants.

| | | | C10-D- | | | Average | |
|---|---|---|---|---|---|---|---|
| | | | Leucinate | Poly- | Lipid/Co- | paricle size | Poly- |
| | Insulin | Solvent | potas- | sorbate | surfactant, | diameter | dispersity |
| No | analogue | 15% | sium | 20 | 51.5% | (nm) | index |
| 1 | 5 mg/ml | Propylene | 3% | 30% | Diglycerol | 5.233 | 0.268 |
| | | glycol | | | monocaprylate | | |
| 2 | 5 mg/ml | Glycerol | 3% | 30% | Diglycerol | 5.424 | 0.27 |
| | | | | | monocaprylate | | |
| 3 | 5 mg/ml | Water | 3% | 30% | Diglycerol | 5.447 | 0.269 |
| | | | | | monocaprylate | | |
| 4 | 5 mg/ml | Propylene | 3% | 30% | Glycerol | 817.3 | 1 |
| | | glycol | | | monocaprylate | | |
| 5 | 5 mg/ml | Glycerol | 3% | 30% | Glycerol | 636.5 | 1 |
| | | | | | monocaprylate | | |
| 6 | 5 mg/ml | Water | 3% | 30% | Glycerol | 610.6 | 1 |
| | | | | | monocaprylate | | |

### Example 4

Liquid formulations comprising insulin, SEDDS, SMEDDS or SNEDDS formulations were prepared according to the guidance given in WO08145728 example 1 and 2, pages 53-54 wherein the FA-Daa according to this invention were added to the isulin solution. Insulin was dissolved in the solvent (propylene glycol, water and/or glycerol), The FA-Daa was then dissolven in said insulin solution whereupon the lipid phase components of SEDDS, SMEDDS or SNEDDS were added to this mixture followed by the surfactants. All formulations contained insulin derivative A14E, B25H, B29K(N(eps)Octadecanedioyl-gGlu-OEG-OEG), desB30 human insulin (5 mg/g). The insulin was first dissolved in water and the pH adjusted to pH 7.4 with a non volatile base (NaOH) followed by freeze drying, the resulting insulin powder was then dissolved first in propylene glycol and then mixed with the other excipients. Liquid formulations comprising insulin, FA-Daa's according to the present invention and SEDDS, SMEDDS or SNEDDS formulations were prepared comprising C12-D-valinate potassium, propylene glycol, diglycerol monocaprylate and different surfactants. Mean particle size (hydrodynamic diameter) was analysed after 10 fold dilution in MilliQ water at 37° C. and respective PDI (polydispersity index). The compositions and the results of particle size analysis are shown in table 6.

TABLE 6
Insulin SEDDS and SMEDDS compositions comprising C12-D-valinate
potassium, propyleneglycol, diglycerol monocaprylate and different surfactants.

| | | | C12-D- | | | Average | |
|---|---|---|---|---|---|---|---|
| | | Pro- | Valine | | Diglycerol | paricle size | Poly- |
| | Insulin | pylene | potas- | Surfactant, | mono- | diameter | dispersity |
| No | analogue | glycol | sium | 30% | caprylate | (nm) | index |
| 1 | 5 mg/ml | 15% | 3% | Tween 20 | 51.5% | 5.047 | 0.266 |
| 2 | 5 mg/ml | 15% | 3% | Tween 40 | 51.5% | 4.835 | 0.279 |
| 3 | 5 mg/ml | 15% | 3% | Tween 60 | 51.5% | 4.872 | 0.274 |
| 4 | 5 mg/ml | 15% | 3% | Tween 80 | 51.5% | 5.408 | 0.287 |
| 5 | 5 mg/ml | 15% | 3% | Span 40 | 51.5% | 25540 | 0.751 |
| 6 | 5 mg/ml | 15% | 3% | Poloxamer | 51.5% | 4.316 | 0.266 |
| | | | | 124 | 51.5% | | |

### Example 5

Liquid formulations comprising insulin, SEDDS, SMEDDS or SNEDDS formulations were prepared according to the guidance given in WO08145728 example 1 and 2, pages 53-54 wherein the FA-Daa according to this invention were added to the isulin solution. Insulin was dissolved in the solvent (propylene glycol, water and/or glycerol), The FA-Daa was then dissolven in said insulin solution whereupon the lipid phase components of SEDDS, SMEDDS or SNEDDS were added to this mixture followed by the surfactants. All formulations contained insulin derivative A1(N,N-Dimethyl), A14E, B1(N, N-dimethyl), B25H, B29K(N(eps)octadecanedioyl-gGlu-OEG-OEG), desB30 human insulin (5 mg/g). The insulin was first dissolved in water and the pH adjusted to pH 7.4 with a non volatile base (NaOH) followed by freeze drying, the resulting insulin powder was then dissolved first in propylene glycol and then mixed with the other excipients as described Liquid formulations comprising insulin, FA-Daa's according to the present invention and SEDDS, SMEDDS or SNEDDS formulations were prepared comprising different fatty acid acylated D-aminoacid salts, propylene glycol, polysorbate 20 and diglycerol monocaprylate. Mean particle size (hydrodynamic diameter) was analysed after 10 fold dilution in MilliQ water at 37° C. and respective PDI (polydispersity index). The compositions and the results of particle size analysis are shown in table 7.

TABLE 7
Insulin SEDDS and SMEDDS compositions comprising different fatty acid acylated D-
aminoacid salts, propylene glycol, polysorbate 20 and diglycerol monocaprylate.

| | | | Fatty acid | | | Average | |
|---|---|---|---|---|---|---|---|
| | | Pro- | acylated D- | Poly- | Diglycerol | paricle size | Poly- |
| | Insulin | pylene | amino acid, | sorbate | mono- | diameter | dispersity |
| No | analogue | glycol | 3% | 20 | caprylate | (nm) | index |
| 1 | 5 mg/ml | 15% | C14-D- | 30% | 51.5% | 59.57 | 0.211 |
| | | | Glutamate | | | | |
| | | | potassium | | | | |
| 2 | 5 mg/ml | 15% | C12-D- | 30% | 51.5% | 12.58 | 0.202 |
| | | | Proline | | | | |
| | | | potassium | | | | |
| 3 | 5 mg/ml | 15% | C12-D- | 30% | 51.5% | 5.086 | 0.264 |
| | | | Leucinate | | | | |
| | | | potassium | | | | |
| 4 | 5 mg/ml | 15% | C12-D- | 30% | 51.5% | 77.7 | 0.332 |
| | | | Alanine | | | | |
| | | | potassium | | | | |
| 5 | 5 mg/ml | 15% | C14-D- | 30% | 51.5% | 73.5 | 1 |
| | | | Tyrosine | | | | |
| | | | potassium | | | | |

### Example 6

An insulin derivative (60 nmol/kg) was dissolved in phosphate buffer (pH 7.4) in presence of fatty acid acylated amino acids. The composition was injected into mid-jejunum of anaesthetized overnight fasted Sprague-Dawley rats (n=6) and the pharmacokinetic profile was obtained by determining concentration of this insulin derivative in plasma samples taken at different time points using ELISA, LOCI or LC-MS protocols.

### Detailed Protocol:

### Rat Pharmacokinecics, Rat PK Following Intraintestinal Injection:

Anaesthetized rats were dosed intraintestinally (into jejunum) with reference compounds and fatty acid acylated D-aminoacid of the invention. Plasma concentrations of insulin analogue as well as changes in blood glucose were measured at specified intervals for 4 hours or more post-dosing. Pharmacokinetic parameters were subsequently calculated using WinNonLin Professional (Pharsight Inc., Mountain View, Calif., USA).

Male Sprague-Dawley rats (Taconic), weighing 250-300 g, fasted for ˜18 h were anesthetized using Hypnorm-Dormicum s.c. (0.079 mg/ml fentanyl citrate, 2.5 mg/ml fluanisone and 1.25 mg/ml midazolam) 2 ml/kg as a priming dose (to timepoint −60 min prior to test substance dosing), 1 ml/kg after 20 min followed by 1 ml/kg every 40 min.

The formulations for the intraintestinal injection model were prepared for example according to the following composition (in weight %):

600 nmol/g A14E, B25H, B29K(N(eps)Octadecanedioyl-gGlu-OEG-OEG), desB30 human insulin

Formulation:

0.15 mM insulin analogue

0.1M fatty acid acylated D-amino acid of the invention

50 mM phosphate buffer pH=8.5

Dose: 60 nmol/kg of insulin

The anesthetized rat was placed on a homeothermic blanket stabilized at 37° C. A 20 cm polyethylene catheter mounted a 1-ml syringe was filled with insulin formulation or vehicle. A 4-5 cm midline incision was made in the abdominal wall. The catheter was gently inserted into mid-jejunum ˜50 cm from the caecum by penetration of the intestinal wall. If intestinal content was present, the application site was moved ±10 cm. The catheter tip was placed approx. 2 cm inside the lumen of the intestinal segment and fixed without the use of ligatures. The intestines were carefully replaced in the abdominal cavity and the abdominal wall and skin were closed with autoclips in each layer. At time 0, the rats were dosed via the catheter, 0.4 ml/kg of test compound or vehicle.

Blood samples for the determination of whole blood glucose concentrations were collected in heparinised 10 μl capillary tubes by puncture of the capillary vessels in the tail tip. Blood glucose concentrations were measured after dilution in 500 μl analysis buffer by the glucose oxidase method using a Biosen autoanalyzer (EKF Diagnostic Gmbh, Germany). Mean blood glucose concentration courses (mean±SEM) were made for each compound.

Samples were collected for determination of the plasma insulin concentration. 100 μl blood samples were drawn into chilled tubes containing EDTA. The samples were kept on ice until centrifuged (7000 rpm, 4° C., 5 min), plasma was pipetted into Micronic tubes and then frozen at 20° C. until assay. Plasma concentrations of the insulin analogue were measured in a immunoassay.

Blood samples were drawn at t=−10 (for blood glucose only), at t=−1 (just before dosing) and at specified intervals for 4 hours or more post-dosing.

Plasma concentration-time profiles were analysed by a non-compartmental pharmacokinetics analysis in WinNonlin 5.2 (Pharsight Inc., Mountain View, Calif., USA). Calculations were performed using individual concentration-time values from each animal. For the calculations of oral bioavailability iv data from previous studies in rats were applied. Results are presented in table 8:

TABLE 8

| Fatty acid acylated D-amino acid | Bioavailability (%) |
|---|---|
| C12-D-Leucine | 7.5 ± 5.4 |
| C16-D-Aspartic acid | 3.2 ± 3.3 |

### Example 7

### Cell Culturing

Caco-2 cells were obtained from the American Type Culture Collection (Manassas, Va.). Cells were seeded in culturing flasks and passaged in Dulbecco's Modified Eagle′ medium supplemented with 10% fetal bovine serum, 1% penicillin/streptomycin (100 U/ml and 100 μg/ml, respectively), 1% L-glutamine and 1% nonessential amino acids. Caco-2 cells were seeded onto tissue culture treated polycarbonate filters in 12-well Transwell plates (1.13 cm2, 0.4 μm pore size) at a density of 10⁵ cells/well. Monolayers were grown in an atmosphere of 5% CO₂-95% O₂ at 37° C. Growth media were replaced every other day. The experiment was performed on day 10-14 after seeding of Caco-2 cells.

Transepithelial Transport

The amount of compound transported from the donor chamber (apical side) to the receiver chamber (basolateral side) was measured. The transport study was initiated by adding 400 μl solution (100 μM of A14E, B25H, B29K(N(eps)Octadecanedioyl-gGlu-OEG-OEG), desB30 human insulin analogue, 100 μM of A14E, B25H, B29K(N(eps)Octadecanedioyl-gGlu-OEG-OEG), desB30 human insulin analogue+0.5 mM fatty acid acylated D-amino acids) and 0.4 ρCi/μl [3H]manntiol in transport buffer to the donor chamber and 1000 μl transport buffer to the receiver chamber. The transport buffer consisted of Hank's balanced saline solution containing 10 mM HEPES, 0.1% adjusted to pH 7.4 after addition of compounds. The transport of [³H]mannitol, a marker for paracellular transport, was measured to verify the integrity of the epithelium.

Before the experiment, the Caco-2 cells were equilibrated for 60 min with transport buffer on both sides of the epithelium. Buffer was then removed and the experiment initiated. Donor samples (20 μl) were taken at 0 min and at the end of the experiment. Receiver samples (200 μl) were taken every 15 min. The study was performed in an atmosphere of 5% CO₂-95% O₂ at 37° C. on a shaking plate (30 rpm).

In all samples with A14E, B25H, B29K(N(eps)Octadecanedioyl-gGlu-OEG-OEG), desB30 human insulin analogue and mannitol, the concentration was determined using a LOCI assay and scintillation counter, respectively.

Before and during the experiment the transepithelial electrical resistance (TEER) of the cell monolayers was monitored. In selected experiments, the transport buffer were changed to culturing medium after end of experiment and the TEER measured 24 h after experiment. The TEER was measured with EVOM™ Epithelial Voltohmmeter connected to Chopsticks.

TABLE 9

| | | Fold | | |
|---|---|---|---|---|
| Compound | Papp* | increase* | TEER 60 min* | TEER 24 h* |
| N-decanoyl-D-leucine | 8.3E−09 | 1.2 | 93.8 | 100.3 |
| N-dodecanoyl-D-Leucine | 4.0E−08 | 6.2 | 48.3 | 101.4 |
| N-dodecanoyl-D-leucine | 1.4E−07 | 14.5 | 42.8 | 100.0 |
| N-dodecanoyl-D- | 8.6E−08 | 21.0 | 37.6 | 108.4 |
| phenylalanine | | | | |
| N-dodecanoyl-D-proline | 8.5E−09 | 1.3 | 76.6 | 99.1 |
| N-dodecanoyl-D-valine | 2.2E−08 | 3.4 | 66.1 | 99.3 |
| N-tetradecanoyl-D-aspartic | 9.2E−09 | 2.2 | 74.2 | 96.1 |
| acid | | | | |
| N-tetradecanoyl-D-glutamic | 6.8E−09 ± 1.8E−09^{¤} | 1.9 ± 0.7^{¤} | 79.9 ± 2.9^{¤} | 102.2 ± 1.9^{¤} |
| acid | | | | |
| N-tetradecanoyl-D-tryptophan | 4.5E−08 | 11.0 | 31.3 | 109.9 |
| N-tetradecanoyl-D-tyrosine | 2.2E−08 | 5.3 | 44.4 | 125.3 |
| N-hexadecanoyl-D-glutamic | 7.4E−08 | 7.7 | 35.6 | 106.2 |
| acid | | | | |
| N-hexadecanoyl-D-aspartic | 2.5E−07 | 26.0 | 24.1 | 112.2 |
| acid | | | | |

*aritmetic average is given for each experiment, for experiments with n = 3 the symbol ¤ is used and standard deviation is given
insulin analogue = A14E, B25H, B29K(N(eps)Octadecanedioyl-gGlu-OEG-OEG), desB30 human insulin

TABLE 10

| | | Fold | | |
|---|---|---|---|---|
| Compound | Papp* | increase* | TEER 60 min* | TEER 24 h* |
| N-decanoyl-L-leucine | 8.3E−09 | 1.2 | 92.2 | 101.8 |
| N-decanoyl-L-Leucine | 2.4E−08 | 3.7 | 58.3 | 102.4 |
| N-dodecanoyl-L-leucine | 6.2E−08 | 6.5 | 47.8 | 107.9 |
| N-dodecanoyl-L-phenylalanine | 8.0E−08 | 19.3 | 36.6 | 100.4 |
| N-dodecanoyl-L-valine | 9.0E−09 | 1.4 | 79.7 | 95.3 |
| N-tetradecanoyl-L-aspartic | 7.6E−09 | 1.8 | 77.1 | 100.2 |
| acid | | | | |
| N-tetradecanoyl-L-glutamic | 7.3E−09 ± 3.4E−09^{¤} | 1.8 ± 0.3^{¤} | 82.0 ± 3.2^{¤} | 103.1 ± 3.7^{¤} |
| acid | | | | |
| N-tetradecanoyl-L-tryptophan | 4.2E−08 | 10.2 | 27.8 | 107.9 |
| N-tetradecanoyl-L-tyrosine | 4.5E−08 | 11.0 | 41.9 | 122.3 |
| N-hexadecanoyl-L-aspartic | 1.2E−07 | 12.3 | 33.2 | 112.3 |
| acid | | | | |
| N-hexadecanoyl-L-glutamic | 1.8E−08 | 1.9 | 72.3 | 109.5 |
| acid | | | | |

*aritmetic average is given for each experiment, for experiments with n = 3 the symbol ¤ is used and standard deviation is given
insulin analogue = A14E, B25H, B29K(N(eps)Octadecanedioyl-gGlu-OEG-OEG), desB30 human insulin

TABLE 11

| | | Fold | TEER* | TEER* |
|---|---|---|---|---|
| Compound | Papp* | increase* | 60 min | 24 h |
| N-hexadecanoyl-L-aspartic | 1.45E−08 | 2.2 | 76.0 | 97.2 |
| acid | | | | |
| N-hexadecanoyl-D-aspartic | 1.38E−08 | 2.1 | 74.7 | 87.0 |
| acid | | | | |
| N-dodecanoyl-L-leucine | 8.79E−08 | 13.3 | 57.4 | 91.7 |
| N-dodecanoyl-D-leucine | 7.62E−08 | 11.6 | 50.9 | 91.4 |

*aritmetic average is given for each experiment, for experiments with n = 3 the symbol ¤ is used and standard deviation is given
insulin analogue A1(Nα,Nα-Dimethyl), A14E, B1(Nα,Nα-dimethyl), B25H, B29K(N(eps)Octadecanedioyl-gGlu-OEG-OEG), desB30 human insulin

TABLE 12
Comparison the D- amino acids, with their L-amino acid counterparts.

| | | Fold | | TEER* |
|---|---|---|---|---|
| Compound | Papp* | increase* | TEER* 60 min | 24 h |
| N-dodecanoyl-L-leucine | 6.20E−08 | 6.5 | 47.8 | 107.9 |
| N-dodecanoyl-D-leucine | 1.40E−07 | 14.5 | 42.8 | 100 |
| N-hexadecanoyl-L-glutamic acid | 1.80E−08 | 1.9 | 72.3 | 109.5 |
| N-hexadecanoyl-D-glutamic acid | 7.40E−08 | 7.7 | 35.6 | 106.2 |
| N-hexadecanoyl-L-aspartic acid | 1.20E−07 | 12.3 | 33.2 | 112.3 |
| N-hexadecanoyl-D-aspartic acid | 2.50E−07 | 26 | 24.1 | 112.2 |
| N-decanoyl-L-leucine | 8.30E−09 | 1.2 | 92.2 | 101.8 |
| N-decanoyl-D-leucine | 8.30E−09 | 1.2 | 93.8 | 100.3 |
| N-tetradecanoyl-L-glutamic acid | 7.3E−09 ± 3.4E−09^{¤} | 1.8 ± 0.3^{¤} | 82.0 ± 3.2^{¤} | 103.1 ± 3.7^{¤} |
| N-tetradecanoyl-D-glutamic acid | 6.8E−09 ± 1.8E−09^{¤} | 1.9 ± 0.7^{¤} | 79.9 ± 2.9^{¤} | 102.2 ± 1.9^{¤} |
| N-tetradecanoyl-L-aspartic acid | 7.60E−09 | 1.8 | 77.1 | 100.2 |
| N-tetradecanoyl-D-aspartic acid | 9.20E−09 | 2.2 | 74.2 | 96.1 |
| N-dodecanoyl-L-valine | 9.00E−09 | 1.4 | 79.7 | 95.3 |
| N-dodecanoyl-D-valine | 2.20E−08 | 3.4 | 66.1 | 99.3 |
| N-decanoyl-L-isoleucine | 2.40E−08 | 3.7 | 58.3 | 102.4 |
| N-dodecanoyl-D-isoleucine | 4.00E−08 | 6.2 | 48.3 | 101.4 |
| N-dodecanoyl-D-proline | 8.50E−09 | 1.3 | 76.6 | 99.1 |
| N-dodecanoyl-L-phenylalanine | 8.0E−08 | 19.3 | 36.6 | 100.4 |
| N-dodecanoyl-D-phenylalanine | 8.6E−08 | 21.0 | 37.6 | 108.4 |
| N-tetradecanoyl-L-tryptophan | 4.2E−08 | 10.2 | 27.8 | 107.9 |
| N-tetradecanoyl-D-tryptophan | 4.5E−08 | 11.0 | 31.3 | 109.9 |
| N-tetradecanoyl-L-tyrosine | 4.5E−08 | 11.0 | 41.9 | 122.3 |
| N-tetradecanoyl-D-tyrosine | 2.2E−08 | 5.3 | 44.4 | 125.3 |

*aritmetic average is given for each experiment, for experiments with n = 3 the symbol ¤ is used and standard deviation is given
insulin analogue A1(Nα,Nα-Dimethyl), A14E, B1(Nα,Nα-dimethyl), B25H, B29K(N(eps)Octadecanedioyl-gGlu-OEG-OEG), desB30 human insulin

### Example 8

Pyridine (604 L, 7.50 mmol) was added dropwise to a mixture of D-tyrosine (414 mg, 2.28 mmol) and trimethylsilyl chloride (1.16 L, 9.12 mmol) in dry dichloromethane (15 mL). The resulting solution was stirred overnight. The solution was cooled to 0 C, and then a solution of myristoyl chloride (680 L, 2.50 mmol) in dry dichloromethane (5 mL) was added dropwise. The cooling bath was removed and the mixture was stirred for 1.5 hr at room temperature. 1 M Hydrochloric acid (20 mL) was added; the mixture was stirred for 15 min and pale yellow solid precipitated. The crystals were filtered off; the filtrate was washed with 1 M hydrochloric acid (3×20 mL), dried over anhydrous sodium sulfate and evaporated to dryness. The residue was combined with previous crystals, dissolved in dichloromethane and crystallized from diethylether (10 mL) and hexanes (15 mL) mixture. The product was filtered off, washed with diethylether and dried in vacuo to give N-myristoyl-D-tyrosine as white crystals.

Yield: 577 mg (59%).

1H NMR spectrum (300 MHz, AcOD-d4, dH): 7.05 (d, J=8.3 Hz, 2H); 6.77 (d, J=8.3 Hz, 2H); 4.89 (dd, J=7.6 and 5.4 Hz, 1H); 3.24-3.07 (m, 2H); 2.97 (dd, J=14.2 and 7.8 Hz, 2H); 2.27 (t, J=7.4 Hz, 2H); 1.67-1.42 (m, 2H); 1.38-1.18 (m, 20H); 0.95-0.82 (m, 3H).

LC-MS purity: 100% (ELSD).

LC-MS Rt (Sunfire 4.6 mm×100 mm, acetonitrile/water 50:50 to 100:0+0.1% FA): 6.44 min.

LC-MS m/z: 391.0 (M+H)+.

The above acid (567 mg, 1.45 mmol) was dissolved in 70% aqueous acetonitrile (40 mL) and neutralized with 0.5 M aqueous solution of sodium hydroxide (2.9 mL). Then the solution was freeze-dried to obtain N-myristoyl-D-tyrosine potassium salt as fine white powder.

### Example 9

2-Chlorotrityl resin 100-200 mesh 1.5 mmol/g (1.48 g, 2.22 mmol) was left to swell in dry dichloromethane (10 mL) for 20 min. A solution of Fmoc-D-Ile-OH (0.52 g, 1.48 mmol) and N,N-diisopropylethylamine (0.98 mL, 5.62 mmol) in dry dichloromethane (5 mL) was added to resin and the mixture was shaken for 4 hrs. Resin was filtered and treated with a solution of N,N-diisopropylethylamine (0.52 mL, 2.96 mmol) in methanol/dichloromethane mixture (4:1, 10 mL, 2×5 min). Then resin was washed with N,N-dimethylformamide (2×10 mL), dichloromethane (2×10 mL) and N,N-dimethylformamide (3×10 mL). Fmoc group was removed by treatment with 20% piperidine in dimethylformamide (1×5 min, 1×30 min, 2×10 mL). Resin was washed with N,N-dimethylformamide (3×10 mL), 2-propanol (2×10 mL) and dichloromethane (20 mL, 2×10 mL). Solution of dodecanoic acid (0.49 g, 2.22 mmol), ethyl cyano-glyoxylate-2-oxime (OXYMA, 0.32 g, 2.22 mmol) 2,4,6-collidine (0.52 mL, 4.00 mmol) and N,N-diisopropylcarbodiimide (0.35 mL, 2.22 mmol) in dichloromethane/N,N-dimethylformamide mixture (4:1, 10 mL) was added to resin and mixture was shaken for 1.5 hr. Resin was filtered and washed with N,N-dimethylformamide (6×10 mL), dichloromethane (6×10 mL), methanol (6×10 mL), dichloromethane (12×10 mL) and diethylether (3×10 mL). The product was cleaved from resin by treatment with a mixture of trifluoroacetic acid:triethylsilane:water (30 mL, 9.25:0.5:0.25) for 30 minutes. Resin was filtered off and washed with trifluoroacetic acid/dichloromethane (1:1, 15 mL) and dichloromethane (5×10 mL). The solvents were removed. The residue was dissolved in toluene (15 mL) and the solvent was removed. This procedure was repeated ten times to remove the traces of trifluoroacetic acid. Crude product was dissolved in dichloromethane (5 mL) and diethylether (70 mL) was added to the solution to precipitate the product which was collected by filtration, washed with diethylether and dried in vacuo to yield title compound as brownish powder.

Yield: 0.51 g (51%).

1H NMR spectrum (300 MHz, CDCl3, dH): 5.96 (d, J=7.7 Hz, 1H); 4.62 (dd, J=8.3, 4.9 Hz, 1H); 2.26 (t, J=7.6 Hz, 2H); 1.72-1.59 (m, 2H); 1.58-1.43 (m, 1H); 1.42-1.14 (m, 18H); 1.02-0.83 (m, 9H).

LC-MS purity: 100% (ELSD).

LC-MS Rt (Sunfire 4.6 mm×100 mm, acetonitrile/water 50:50 to 100:0+0.1% FA): 6.66 min. LC-MS m/z: 314.0 (M+H)+.

N-Lauroyl-D-Leucine (0.51 g, 1.62 mmol) was dissolved in 70% aqueous acetonitrile (20 mL) and neutralized with 0.1 M aqueous solution of potassium hydroxide (16 mL). Then the solution was freeze-dried to obtain the title compound as fine yellow powder.

### Example 10

Pyridine (2.00 mL, 0.03 mol) was added dropwise to a mixture of D-proline (0.50 g, 4.30 mmol) and trimethylsilyl chloride (3.20 mL, 0.03 mol) in dry dichloromethane (15 mL) over 10 min. The resulting mixture was stirred for 1 hr. The suspension was cooled to 0 C, and then a solution of lauroyl chloride (0.86 mL, 3.70 mmol) in dry dichloromethane (2 mL) was added dropwise over 20 min. The cooling batch was removed and the mixture was stirred for 1.5 hrs at room temperature. 1 M Hydrochloric acid (15 mL) was added, the mixture was stirred for 15 min, then ethylacetate (50 mL) was added and the phases were separated. The organic layer was washed with 1 M hydrochloric acid (3×20 mL), dried over anhydrous magnesium sulfate and evaporated to dryness. The residue was crystallized from ethylacetate (15 mL) and hexanes (150 mL) mixture. The crystals were filtered off, washed with hexanes and dried in vacuo to give N-lauroyl-D-proline as white crystals.

Yield: 1.09 g (99%).

1H NMR spectrum (300 MHz, CDCl3, dH): 4.69-4.55 (m, 1H); 3.65-3.38 (m, 2H); 2.55-2.31 (m, 2H); 2.07-1.96 (m, 2H); 1.75-1.60 (m, 2H); 1.39-1.14 (m, 16H); 0.93-0.80 (m, 3H).

LC-MS purity: 100% (ELSD).

LC-MS Rt (Sunfire 4.6 mm×100 mm, acetonitrile/water 50:50 to 100:0+0.1% FA): 6.06 min.

LC-MS m/z: 299.0 (M+H)+.

N-lauroyl-D-proline (1.08 g, 3.60 mmol) was dissolved in 70% aqueous acetonitrile (20 mL) and neutralized with 0.1 M aqueous solution of potassium hydroxide (36 mL). Then the solution was freeze-dried to obtain the title compound as fine brownish oil.

### Example 11

Pyridine (2.00 mL, 0.03 mol) was added dropwise to a mixture of D-valine (0.50 g, 4.30 mmol) and trimethylsilyl chloride (3.20 mL, 0.03 mol) in dry dichloromethane (15 mL) over 10 min. The resulting mixture was stirred for 1 hr. The suspension was cooled to 0 C, and then a solution of lauroyl chloride (0.86 mL, 3.70 mmol) in dry dichloromethane (2 mL) was added dropwise over 20 min. The cooling batch was removed and the mixture was stirred for 1.5 hrs at room temperature. 1 M Hydrochloric acid (15 mL) was added, the mixture was stirred for 15 min, then ethylacetate (50 mL) was added and the phases were separated. The organic layer was washed with 1 M hydrochloric acid (3×20 mL), dried over anhydrous magnesium sulfate and evaporated to dryness. The residue was crystallized from ethylacetate (15 mL) and hexanes (150 mL) mixture. The crystals were filtered off, washed with hexanes and dried in vacuo to give N-lauroyl-D-valine as white crystals.

Yield: 1.20 g (99%).

1H NMR spectrum (300 MHz, CDCl3, dH): 5.94 (d, J=8.3 Hz, 1H), 4.58 (dd, J=8.5, 4.9 Hz, 1H); 2.33-2.17 (m, 3H); 1.73-1.56 (m, 2H); 1.41-1.17 (m, 16H); 0.99 (dd, J=10.2, 6.8 Hz, 6H), 0.92-0.83 (m, 3H).

LC-MS purity: 100% (ELSD).

LC-MS Rt (Sunfire 4.6 mm×100 mm, acetonitrile/water 50:50 to 100:0+0.1% FA): 6.26 min.

LC-MS m/z: 300.0 (M+H)+.

N-Lauroyl-D-valine (1.19 g, 3.98 mmol) was dissolved in 70% aqueous acetonitrile (20 mL) and neutralized with 0.1 M aqueous solution of potassium hydroxide (40 mL). Then the solution was freeze-dried to obtain the title compound as fine white powder.

### Example 12

2-Chlorotrityl resin 100-200 mesh 1.5 mmol/g (2.34 g, 3.51 mmol) was left to swell in dry dichloromethane (40 mL) for 40 min. A solution of Fmoc-DArg(Pbf)-OH (746 mg, 1.15 mmol) and N,N-diisopropylethylamine (775 L, 4.44 mmol) in dry dichloromethane (35 mL) was added to resin and the mixture was shaken for 16 hrs. Resin was filtered and treated with a solution of N,N-diisopropylethylamine (405 L, 2.34 mmol) in methanol/dichloromethane mixture (4:1, 35 mL, 5 min). Then resin was washed with dichloromethane (2×35 mL) and N,N-dimethylformamide (2×35 mL). Fmoc group was removed by treatment with 20% piperidine in N,N-dimethylformamide (2×35 mL, 1×5 min, 1×20 min). Resin was washed with N,N-dimethylformamide (2×35 mL), 2-propanol (2×35 mL), dichloromethane (2×35 mL) and N,N-dimethylformamide (2×35 mL). Solution of lauric acid (691 mg, 3.45 mmol), 2-(6-chloro-1H-benzotriazole-1-yl)-1,1,3,3-tetramethylaminium hexafluorophosphate (HCTU, 1.43 g, 3.45 mmol) and N,N-diisopropylethylamine (1.08 mL, 6.21 mmol) in the mixture of N,N-dimethylformamide (10 mL) and dichloromethane (25 mL) was added to resin and mixture was shaken for 6 hrs. Resin was filtered and washed with dichloromethane (2×35 mL), N,N-dimethylformamide (2×35 mL), methanol (2×35 mL) and dichloromethane (10×35 mL). The product was cleaved from the resin by the treatment with trifluoroacetic acid/triethylsilane/water (35 mL, 95:3:2) for 2 hrs. Resin was filtered off and washed with trifluoroacetic acid (3×30 mL) and dichloromethane (3×30 mL). The solvent was removed under reduced pressure. The residue was treated with diethylether (5 mL) and hexanes (20 mL). Product was collected by filtration, washed with hexanes and dried in vacuo to yield N-lauroyl-D-arginine as off-white solid.

Yield: 283 mg (69%).

1H NMR spectrum (300 MHz, AcOD-d4, dH): 4.72-4.55 (m, 1H); 3.38-3.16 (m, 2H); 2.45-2.23 (m, 2H); 2.12-1.55 (m, 6H); 1.41-1.16 (m, 16H); 0.95-0.82 (m, 3H).

LC-MS purity: 98% (ELSD).

LC-MS Rt (Sunfire 4.6 mm×100 mm, acetonitrile/water 05:95 to 100:0+0.1% TFA): 6.69 min.

LC-MS m/z: 356.0 (M+H).

The above acid (277 mg, 0.78 mmol) was dissolved in 70% aqueous acetonitrile (40 mL) and neutralized with 0.5 M aqueous solution of sodium hydroxide (3.1 mL). Then the solution was freeze-dried to obtain N-lauroyl-D-arginine potassium salt as fine white powder.

### Example 13

Pyridine (478 L, 5.94 mmol) was added dropwise to a mixture of D-tryptophan (404 mg, 1.98 mmol) and trimethylsilyl chloride (754 L, 5.94 mmol) in dry dichloromethane (15 mL). The resulting suspension was stirred for 4 hrs until a clear solution was formed. The solution was cooled to 0 C, and then a solution of myristoyl chloride (489 L, 1.80 mmol) in dry dichloromethane (15 mL) was added dropwise. The cooling bath was removed and the mixture was stirred for 1.5 hr at room temperature. 1 M Hydrochloric acid (20 mL) was added, the mixture was stirred for 15 min, than the phases were separated. The organic layer was washed with 1 M hydrochloric acid (3×20 mL), dried over anhydrous sodium sulfate and evaporated to dryness. The residue was crystallized from dichloromethane (5 mL) and hexanes (15 mL) mixture. The crystals were filtered off, washed with hexanes and dried in vacuo to give N-myristoyl-D-tryptophan as white crystals.

Yield: 594 mg (80%).

1H NMR spectrum (300 MHz, CDCl3, dH): 8.34 (bs, 1H); 7.58 (d, J=7.9 Hz, 1H); 7.35 (d, J=7.7 Hz, 1H); 7.25-7.07 (m, 2H); 7.01 (s, 1H); 6.08 (d, J=7.5 Hz, 1H); 4.99-4.85 (m, 1H); 3.45-3.25 (m, 2H); 2.11 (t, J=7.6 Hz, 2H); 1.62-1.41 (m, 2H); 1.32-1.14 (m, 20H); 0.89 (t, J=6.4 Hz, 3H).

LC-MS purity: 100% (ELSD).

LC-MS Rt (Sunfire 4.6 mm×100 mm, acetonitrile/water 50:50 to 100:0+0.1% FA): 7.21 min.

LC-MS m/z: 414.0 (M+H)+.

The above acid (585 mg, 1.40 mmol) was dissolved in 70% aqueous acetonitrile (40 mL) and neutralized with 0.5 M aqueous solution of potassium hydroxide (2.80 mL). Then the solution was freeze-dried to obtain N-myristoyl-D-tryptophan potassium salt as fine white powder.

### Example 14

Pyridine (1.85 mL, 0.02 mol) was added dropwise to a mixture of D-aspartic acid (0.50 g, 3.80 mmol) and trimethylsilyl chloride (2.80 mL, 0.03 mol) in dry dichloromethane (15 mL) over 10 min. The resulting mixture was stirred for 1 hr. The suspension was cooled to 0 C, and then a solution of myristoyl chloride (0.81 mL, 3.30 mmol) in dry dichloromethane (2 mL) was added dropwise over 20 min. The cooling batch was removed and the mixture was stirred for 1.5 hrs at room temperature. 1 M Hydrochloric acid (15 mL) was added, the mixture was stirred for 15 min, then ethylacetate (50 mL) was added and the phases were separated. The organic layer was washed with 1 M hydrochloric acid (3×20 mL), dried over anhydrous magnesium sulfate and evaporated to dryness. The residue was crystallized from ethylacetate (15 mL) and hexanes (150 mL) mixture. The crystals were filtered off, washed with hexanes and dried in vacuo to give N-myristoyl-D-aspartic acid as white crystals.

Yield: 0.70 g (62%).

1H NMR spectrum (300 MHz, AcOD-d4, dH): 4.99 (d, J=5.0 Hz, 1H), 3.20-2.90 (m, 2H); 2.35 (t, J=7.40, 2 H); 1.71-1.54 (m, 2H); 1.29 (m, 20H); 0.89 (t, J=6.4, 3 H).

LC-MS purity: 95% (ELSD).

LC-MS Rt (Sunfire 4.6 mm×100 mm, acetonitrile/water 50:50 to 100:0+0.1% FA): 6.32 min.

LC-MS m/z: 344.0 (M+H)+.

N-Myristoyl-D-aspartic acid (0.70 g, 2.04 mmol) was dissolved in 70% aqueous acetonitrile (20 mL) and neutralized with 0.1 M aqueous solution of potassium hydroxide (41 mL). Then the solution was freeze-dried to obtain the title compound as fine white powder.

### Example 15

Pyridine (1.40 mL, 0.02 mol) was added dropwise to a mixture of D-phenylalanine (0.50 g, 3.03 mmol) and trimethylsilyl chloride (2.25 mL, 0.02 mol) in dry dichloromethane (15 mL) over 10 min. The resulting mixture was stirred for 1 hr. The suspension was cooled to 0 C, and then a solution of lauroyl chloride (0.61 mL, 2.64 mmol) in dry dichloromethane (2 mL) was added dropwise over 20 min. The cooling batch was removed and the mixture was stirred for 1.5 hrs at room temperature. 1 M Hydrochloric acid (15 mL) was added, the mixture was stirred for 15 min, then ethylacetate (50 mL) was added and the phases were separated. The organic layer was washed with 1 M hydrochloric acid (3×20 mL), dried over anhydrous magnesium sulfate and evaporated to dryness. The residue was crystallized from ethylacetate (15 mL) and hexanes (150 mL) mixture. The crystals were filtered off, washed with hexanes and dried in vacuo to give N-lauroyl-D-phenylalanine as white crystals.

Yield: 0.86 g (93%).

1H NMR spectrum (300 MHz, AcOD-d4, dH): 7.36-7.15 (m, 5H), 4.96 (dd, J=8.0, 5.2 Hz, 1H); 3.35-2.98 (m, 2H); 2.26 (t, J=7.4 Hz, 2H); 1.64-1.42 (m, 2H); 1.29 (bs, 16H); 0.95-0.84 (m, 3H).

LC-MS purity: 95% (ELSD).

LC-MS Rt (Sunfire 4.6 mm×100 mm, acetonitrile/water 50:50 to 100:0+0.1% FA): 6.50 min.

LC-MS m/z: 349.0 (M+H)+.

N-Lauroyl-D-phenylalanine (0.86 g, 2.50 mmol) was dissolved in 70% aqueous acetonitrile (20 mL) and neutralized with 0.1 M aqueous solution of potassium hydroxide (25 mL). Then the solution was freeze-dried to obtain the title compound as fine white powder.

### Example 16

Pyridine (1.70 mL, 0.02 mol) was added dropwise to a mixture of D-glutamic acid (0.50 g, 3.50 mmol) and trimethylsilyl chloride (2.60 mL, 0.02 mol) in dry dichloromethane (15 mL) over 10 min. The resulting mixture was stirred for 1 hr. The suspension was cooled to 0 C, and then a solution of myristoyl chloride (0.74 mL, 3.00 mmol) in dry dichloromethane (2 mL) was added dropwise over 20 min. The cooling batch was removed and the mixture was stirred for 1.5 hrs at room temperature. 1 M Hydrochloric acid (15 mL) was added, the mixture was stirred for 15 min, then ethylacetate (50 mL) was added and the phases were separated. The organic layer was washed with 1 M hydrochloric acid (3×20 mL), dried over anhydrous magnesium sulfate and evaporated to dryness. The residue was crystallized from ethylacetate (15 mL) and hexanes (150 mL) mixture. The crystals were filtered off, washed with hexanes and dried in vacuo to give N-myristoyl-D-glutamic acid as white crystals.

Yield: 0.92 g (86%).

1H NMR spectrum (300 MHz, CDCl3, dH): 6.32-6.16 (m, 1H); 4.73-4.59 (m, 1H), 2.65-2.43 (m, 2H); 2.32-2.10 (m, 4H); 1.74-1.57 (m, 2H); 1.30 (bs, 20H); 0.95-0.82 (m, 3H).

LC-MS purity: 100% (ELSD).

LC-MS Rt (Sunfire 4.6 mm×100 mm, acetonitrile/water 50:50 to 100:0+0.1% FA): 5.97 min.

LC-MS m/z: 358.0 (M+H)+.

N-Myristoyl-D-glutamic acid (0.92 g, 2.56 mmol) was dissolved in 70% aqueous acetonitrile (20 mL) and neutralized with 0.1 M aqueous solution of potassium hydroxide (51 mL). Then the solution was freeze-dried to obtain the title compound as fine white powder.

### Example 17

Pyridine (2.60 mL, 0.03 mol) was added dropwise to a mixture of D-alanine (0.50 g, 6.00 mmol) and trimethylsilyl chloride (4.20 mL, 0.03 mol) in dry dichloromethane (15 mL) over 10 min. The resulting mixture was stirred for 1 hr. The suspension was cooled to 0 C, and then a solution of lauroyl chloride (1.30 mL, 5.00 mmol) in dry dichloromethane (2 mL) was added dropwise over 20 min. The cooling batch was removed and the mixture was stirred for 1.5 hrs at room temperature. 1 M Hydrochloric acid (15 mL) was added, the mixture was stirred for 15 min, then ethylacetate (50 mL) was added and the phases were separated. The organic layer was washed with 1 M hydrochloric acid (3×20 mL), dried over anhydrous magnesium sulfate and evaporated to dryness. The residue was crystallized from ethylacetate (15 mL) and hexanes (150 mL) mixture. The crystals were filtered off, washed with hexanes and dried in vacuo to give N-lauroyl-D-alanine as white crystals.

Yield: 0.57 g (42%).

1H NMR spectrum (300 MHz, CDCl3, dH): 5.91 (d, J=5.7 Hz, 1H), 4.66-4.51 (m, 1H); 2.25 (t, J=7.5, 2 H); 1.71-1.58 (m, 2H); 1.48 (d, J=7.2 Hz, 3H); 1.37-1.18 (m, 16H), 0.95-0.82 (m, 3H).

LC-MS purity: 100% (ELSD).

LC-MS Rt (Sunfire 4.6 mm×100 mm, acetonitrile/water 35:65 to 100:0+0.1% FA): 7.34 min.

LC-MS m/z: 272.0 (M+H)+.

N-Lauroyl-D-alanine (0.57 g, 2.1 mmol) was dissolved in 70% aqueous acetonitrile (20 mL) and neutralized with 0.1 M aqueous solution of potassium hydroxide (21 mL). Then the solution was freeze-dried to obtain the title compound as fine white powder.

### Example 18

Pyridine (1.65 mL, 0.02 mol) was added dropwise to a mixture of D-glutamic acid (0.5 g, 3.5 mmol) and trimethylsilyl chloride (2.6 mL, 0.02 mol) in dry dichloromethane (10 mL) over 10 min. The resulting mixture was stirred for 1 hour. The suspension was cooled to 0 C, and then a solution of palmitoyl chloride (0.92 mL, 3 mmol) in dry dichloromethane (1.3 mL) was added dropwise over 20 min. The cooling batch was removed and the mixture was stirred for 1.5 hrs at room temperature. 1 M Hydrochloric acid (15 mL) was added, the mixture was stirred for 15 min, then ethylacetate (50 mL) was added and the phases were separated. The organic layer was washed with 1 M hydrochloric acid (3×15 mL), dried over anhydrous magnesium sulfate and evaporated to dryness. The residue was crystallized from ethylacetate (15 mL) and hexanes (100 mL) mixture. The crystals were filtered off, washed with hexanes and dried in vacuo to give N-palmitoyl-D-glutamic acid as white crystals.

Yield: 1.04 g (90%).

RF (SiO2, dichloromethane/methanol 80:20): 0.14.

1H NMR spectrum (300 MHz, AcOD-d4, 80 C, dH): 4.76-4.64 (m, 1H); 2.62-2.08 (m, 6H); 1.73-1.58 (m, 2H); 1.32 (s, 24H); 0.95-0.85 (m, 3H).

LC-MS purity: 100% (ELSD).

LC-MS Rt (Sunfire 4.6 mm×100 mm, acetonitrile/water 50:50 to 100:0+0.1% FA): 7.18 min.

LC-MS m/z: 386.0 (M+H)+.

The above acid (1.04 g, 2.70 mmol) was dissolved in 70% aqueous acetonitrile (40 mL) and neutralized with 0.1 M aqueous solution of potassium hydroxide (54 mL). Then the solution was freeze-dried to obtain Palmitoyl-DGlu(OK)-OK as fine white powder.

### Example 19

Pyridine (1.79 mL, 0.02 mol) was added dropwise to a mixture of L-aspartic acid (0.5 g, 3.8 mmol) and trimethylsilyl chloride (2.8 mL, 0.02 mol) in dry dichloromethane (10 mL) over 10 min. The resulting mixture was stirred for 1 hour. The suspension was cooled to 0 C, and then a solution of palmitoyl chloride (1 mL, 3.3 mmol) in dry dichloromethane (1.3 mL) was added dropwise over 20 min. The cooling batch was removed and the mixture was stirred for 1.5 hrs at room temperature. 1 M Hydrochloric acid (15 mL) was added, the mixture was stirred for 15 min, then ethylacetate (50 mL) was added and the phases were separated. The organic layer was washed with 1 M hydrochloric acid (3×15 mL), dried over anhydrous magnesium sulfate and evaporated to dryness. The residue was crystallized from ethylacetate (15 mL) and hexanes (100 mL) mixture. The crystals were filtered off, washed with hexanes and dried in vacuo to give N-palmitoyl-D-aspartic acid as white crystals.

Yield: 1.05 g (85%).

RF (SiO2, dichloromethane/methanol 80:20): 0.10.

1H NMR spectrum (300 MHz, AcOD-d4, 80 C, dH): 4.99 (t, J=4.99 Hz, 1H); 3.15-2.92 (m, 2H); 2.41-2.29 (m, 2H); 1.72-1.55 (m, 2H); 1.29 (s, 24H); 0.94-0.83 (m, 3H).

LC-MS purity: 100% (ELSD).

LC-MS Rt (Sunfire 4.6 mm×100 mm, acetonitrile/water 50:50 to 100:0+0.1% FA): 7.93 min.

LC-MS m/z: 372.0 (M+H)+.

The above acid (1.05 g, 2.82 mmol) was dissolved in 70% aqueous acetonitrile (40 mL) and neutralized with 0.1 M aqueous solution of potassium hydroxide (56.4 mL). Then the solution was freeze-dried to obtain Palmitoyl-DAsp(OK)-OK as white fine powder.

### Example 20

Pyridine (0.92 mL, 0.01 mol) was added dropwise to a mixture of D-Leucine (0.5 g, 3.8 mmol) and trimethylsilyl chloride (1.45 mL, 0.01 mol) in dry dichloromethane (11 mL) over 10 min. The resulting mixture was stirred for 1 hour. The suspension was cooled to 0 C, and then a solution of lauroyl chloride (0.8 mL, 3.5 mmol) in dry dichloromethane (1.4 mL) was added dropwise over 20 min. The cooling batch was removed and the mixture was stirred for 1.5 hrs at room temperature. 1 M Hydrochloric acid (14 mL) was added, the mixture was stirred for 15 min and the phases were separated. The organic layer was washed with 1 M hydrochloric acid (3×10 mL), dried over anhydrous magnesium sulfate and evaporated to dryness. The residue was crystallized from dichloromethane (15 mL) and hexanes (100 mL) mixture. The crystals were filtered off, washed with hexanes and dried in vacuo to give N-lauroyl-D-leucine as white crystals.

Yield: 0.86 g (78%).

RF (SiO2, dichloromethane/methanol 80:20): 0.38.

1H NMR spectrum (300 MHz, AcOD-d4, 80 C, dH): 4.73-4.71 (m, 1H); 2.33 (t, J=7.44 Hz, 2H); 1.80-1.57 (m, 5H); 1.32 (br.s, 16H); 1.03-0.94 (m, 6H); 0.94-0.83 (m, 3H).

LC-MS purity: 100% (ELSD).

LC-MS Rt (Sunfire 4.6 mm×100 mm, acetonitrile/water 35:65 to 100:0+0.1% FA): 8.31 min.

LC-MS m/z: 314.0 (M+H)+.

Nlauroyl-D-leucine (0.86 g, 2.7 mmol) was dissolved in 70% aqueous acetonitrile (20 mL) and neutralized with 0.1 M aqueous solution of potassium hydroxide (27 mL). Then the solution was freeze-dried to obtain Nlauroyl-DLeu-OK as fine white powder.

### Example 21

Pyridine (0.92 mL, 0.01 mol) was added dropwise to a mixture of D-Leucine (0.5 g, 3.8 mmol) and trimethylsilyl chloride (1.45 mL, 0.01 mol) in dry dichloromethane (11 mL) over 10 min. The resulting mixture was stirred for 1 hour. The suspension was cooled to 0 C, and then a solution of decanoyl chloride (0.7 mL, 3.5 mmol) in dry dichloromethane (1.4 mL) was added dropwise over 20 min. The cooling batch was removed and the mixture was stirred for 1.5 hrs at room temperature. 1 M Hydrochloric acid (14 mL) was added, the mixture was stirred for 15 min and the phases were separated. The organic layer was washed with 1 M hydrochloric acid (3×10 mL), dried over anhydrous magnesium sulfate and evaporated to dryness. The residue was crystallized from dichloromethane (15 mL) and hexanes (100 mL) mixture. The crystals were filtered off, washed with hexanes and dried in vacuo to give N-decanoyl-D-leucine as white crystals.

Yield: 0.67 g (67%).

RF (SiO2, dichloromethane/methanol 80:20): 0.21.

1H NMR spectrum (300 MHz, AcOD-d4, 80 C, dH): 4.75-4.59 (m, 1H); 2.39-2.26 (m, 2H); 1.86-1.56 (m, 5H); 1.32 (br.s, 12H); 1.03-0.83 (m, 9H).

LC-MS purity: 100% (ELSD).

LC-MS Rt (Sunfire 4.6 mm×100 mm, acetonitrile/water 35:65 to 100:0+0.1% FA): 7.17 min.

LC-MS m/z: 286.0 (M+H)+.

Ndecanoyl-D-leucine (0.66 g, 2.3 mmol) was dissolved in 70% aqueous acetonitrile (20 mL) and neutralized with 0.1 M aqueous solution of potassium hydroxide (23 mL). Then the solution was freeze-dried to obtain N-decanoyl-DLeu-OK as fine white powder.

### Example 22

The composition of the insulin degludec/liraglutide drug product that Novo Nordisk A/S currently has in clinical development is shown below. This formulation has been shown to be a stable combination product suitable for use in type II diabetes clinical trials (subcutaneous infection).”

### Names of Ingredients in the Drug Product Formulation

### Drug Substances

Liraglutide, 3.6 mg (960 nmol) per ml
Insulin degludec, 600 nmol (100 U) per ml

### Excipients

Phenol
Glycerol
Zinc

### Formulation Process Specialities

Both insulin degludec and liraglutide drug substances are added in the form of a solid powder, separately and directly to a mixture of excipients.
All of the zinc is added in one step.
There is no need for holding time anywhere in the formulation process.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A pharmaceutical composition comprising
a. At least one FA-Daa or a salt thereof represented by the general formula A-Xy, wherein A is a non-polar uncharged or acidic amino acid and Xy is a fatty acid moiety attached by acylation to A's alpha amino group and y represents the number of carbon atoms in said fatty acid moiety, wherein y is 10, 12, 14, 16 or 18 when said amino acid is a non-polar uncharged amino acid and y is 16 or 18 when said amino acid is an acidic, wherein the stereo configuration of the chiral carbon atom in the amino acid moiety is D and
b. a hydrophilic peptide or protein.

2. The pharmaceutical composition according to claim 1, wherein said pharmaceutical composition is an oral pharmaceutical composition.

3. The oral pharmaceutical composition according to claim 2, wherein said salt is selected from the group consisting of sodium (Na+) and potassium (K+).

4. The oral pharmaceutical composition according to claim 2, wherein y is 12.

5. The oral pharmaceutical composition according to claim 2, wherein y is 14.

6. The oral pharmaceutical composition according to claim 2, wherein y is 16.

7. The oral pharmaceutical composition according to claim 2, wherein y is 18.

8. The oral composition according to claim 2, wherein said hydrophilic peptide or protein is insulin.

9. The oral pharmaceutical composition according to claim 2, wherein the amino acid residue of said FA-Daa is selected from the group consisting of: Sodium or potassium lauroyl D-alaninate, N-dodecanoyl-D-alanine, Sodium or potassium myristoyl D-Alaninate, N-tetradecanoyl D-Alanine, Sodium or potassium palmitoyl D-Alaninate, N-hexadecanoyl D-Alanine, Sodium or potassium stearoyl D-Alaninate, N-octadecanoyl D-Alanine, Sodium or potassium lauroyl D-Isoleucinate, N-dodecanoyl-D-Isoleucine, Sodium or potassium myristoyl D-Isoleucinate, N-tetradecanoyl D-Isoleucine, Sodium or potassium palmitoyl D-Isoleucinate, N-hexadecanoyl D-Isoleucine, Sodium or potassium stearoyl D-Isoleucinate, N-octadecanoyl D-Isoleucine, Sodium or potassium lauroyl D-Leucinate, N-dodecanoyl-D-Leucine, Sodium or potassium myristoyl D-Leucinate, N-tetradecanoyl D-Leucine, Sodium or potassium palmitoyl D-Leucinate, N-hexadecanoyl D-Leucine, Sodium or potassium stearoyl D-Leucinate, N-octadecanoyl D-Leucine, Sodium or potassium lauroyl D-Prolinate, N-dodecanoyl-D-Proline, Sodium or potassium myristoyl D-Prolinate, N-tetradecanoyl D-Proline, Sodium or potassium palmitoyl D-Prolinate, N-hexadecanoyl D-Proline, Sodium or potassium stearoyl D-Prolinate, N-octadecanoyl D-Proline, Sodium or potassium lauroyl D-Valinate, N-dodecanoyl-D-Valine, Sodium or potassium myristoyl D-Valinate, N-tetradecanoyl D-Valine, Sodium or potassium palmitoyl D-Valinate, N-hexadecanoyl D-Valine, Sodium or potassium stearoyl D-Valinate, N-octadecanoyl D-Valine Sodium or potassium palmitoyl D-Aspartate, N-hexadecanoyl D-Aspartic acid, Sodium or potassium palmitoyl D-Glutamate, N-hexadecanoyl D-Glutamic acid, Sodium or potassium stearoyl D-Aspartate, N-octadecanoyl D-Aspartic acid, Sodium or potassium stearoyl D-Glutamate and N-octadecanoyl D-Glutamic acid.

10. The oral pharmaceutical composition according to claim 2, further comprising propylene glycol.

11. The oral pharmaceutical composition according to claim 2, further comprising SEDDS, SMEDDS or SNEDDS.

12. The pharmaceutical composition according to claim 1, which comprises less than 10%(w/w) water.

13. (canceled)

14. A method for increasing bioavailability of an insulin, insulin peptide or protein or insulin analouges or derivatives comprising the steps of including a FA-aa in a pharmaceutical composition of an insulin, insulin peptide or protein or insulin analouges or derivatives administered to an individual.

15. A method for the manufacture of the pharmaceutical composition according to claim 1, comprising the step of mixing said FA-Daa to a mixture of an insulin peptide or protein and the ingredients for SEDDS, SMEDDS or SNEDDS.
